(19) United States
(12) Patent Application Publication
(10) Pub. No.: US 2024/0115703 A1
(43) Pub. Date: Apr. 11, 2024

(54) GENETICALLY ENGINEERED ANTI-CD19 CAR-T CELLS FOR USE IN TREATING B-CELL MALIGNANCIES

(71) Applicant: CRISPR Therapeutics AG, Zug (CH)

(72) Inventors: Ziliang LI, Cambridge, MA (US); Annie Yang WEAVER, Cambridge, MA (US); Sarah COHEN, Cambridge, MA (US); Ewelina MORAWA, Cambridge, MA (US)

(21) Appl. No.: 18/482,752

(22) Filed: Oct. 6, 2023

Related U.S. Application Data
(60) Provisional application No. 63/478,262, filed on Jan. 3, 2023.
(60) Provisional application No. 63/414,788, filed on Oct. 10, 2022.

(51) Int. Cl. A61K 39/00; A61P 35/00; A61P 35/02

(52) U.S. Cl. CPC A61K 39/4611; A61K 39/4631; A61K 39/464412; A61P 35/00; A61P 35/02; A61K 2239/38; A61K 2239/39; A61K 2239/48

(57) ABSTRACT

Methods for treating B-cell malignancies such as relapsed and/or refractory B-cell malignancies with a population of genetically engineered T cells expressing a chimeric antigen receptor (CAR) targeting CD19 and having multiple genetic edits, including a disrupted TRAC gene, a disrupted β2M gene, a disrupted Regnase 1 gene, and/or a disrupted TGFBRII gene.

### CROSS-REFERENCE TO RELATED APPLICATIONS

This application claims the benefit under 35 U.S.C. § 119(e) of U.S. Provisional Patent Application No. 63/478,262, filed Jan. 3, 2023, and U.S. Provisional Patent Application No. 63/414,788, filed Oct. 10, 2022, the entire contents of each of which are incorporated by reference herein.

### SEQUENCE LISTING

The instant application contains a substitute Sequence Listing that has been filed electronically in XML format and is hereby incorporated by reference in its entirety. Said XML copy, created on Oct. 30, 2023, is named “095136-0787-076US1_SUBSEQ.xml” and is 103,730 bytes in size, which replaces the Sequence Listing co-filed with the application on Oct. 6, 2023, created on Sep. 29, 2023, named “095136-0787-076US1_SEQ.xml” and 101,738 bytes in size.

### BACKGROUND OF THE INVENTION

Chimeric antigen receptor (CAR) T-cell therapy uses genetically modified T cells to more specifically and efficiently target and kill cancer cells. After T cells have been collected from the blood, the cells are engineered to include CARs on their surface. The CARs may be introduced into the T cells using CRISPR/Cas9 gene editing technology. When these allogeneic CAR T cells are injected into a patient, the receptors enable the T cells to kill cancer cells.

T cells having improved persistence in culture are desired in CAR T therapy. Such T cells live longer in both in vitro and in vivo, thereby conferring benefits in CAR T cell manufacturing and clinical applications. However, it remains challenging to improve persistence of T cells in culture.

### SUMMARY OF THE INVENTION

The present disclosure is based, at least in part, on the development of allogeneic cell therapy for relapsed and/or refractory B cell malignancies using genetically engineered T cells (e.g., CTX112 cells) expressing an anti-CD19 chimeric antigen receptor (CAR) and having multiple genetic modifications, including a disrupted TRAC gene, a disrupted B2M gene, a disrupted beta-2-microglobulin (TGFBRII) gene, and a disrupted Regnase-1 (Reg1) gene.

In some aspects, the present disclosure provides a method for treating a B-cell malignancy, comprising: (i) subjecting a human patient having a B-cell malignancy to a lymphodepletion (LD) treatment; and (ii) administering to the human patient a first dose of a population of genetically engineered T cells after step (i). The population of genetically engineered T cells comprising T cells that comprise: (a) a disrupted T cell receptor alpha chain constant region (TRAC) gene, (b) a disrupted beta-2-microglobulin (β2M) gene, (c) a disrupted Regnase-1 (Reg1) gene, (d) a disrupted Transforming Growth Factor Beta Receptor II (TGFBRII) gene, and (e) a nucleic acid encoding a chimeric antigen receptor (CAR) that binds human CD19 (anti-CD19 CAR). In some instances,

In some embodiments, the anti-CD19 CAR comprises a single chain variable fragment (scFv) that binds CD19 (anti-CD19 scFv), a co-stimulatory domain of CD28, and a CD3ζ cytoplasmic signaling domain. The anti-CD19 scFv may comprise a heavy chain variable region (V_{H}) that comprises the same heavy chain complementary determining regions (CDRs) as those in SEQ ID NO: 81; and (ii) a light chain variable region (V_{L}) that comprises the same light chain CDRs as those in SEQ ID NO: 82. In some instances, the nucleic acid encoding the anti-CD19 CAR is inserted at the disrupted TRAC gene.

In some embodiments, the first dose of the population of genetically engineered T cells is about 1.0×10⁷ to about 6.0×10⁸ CAR⁺ T cells. In some examples, the first dose of the population of genetically engineered T cells is about 3.0×10⁷ CAR⁺ T cells. In some examples, the first dose of the population of genetically engineered T cells is about 1.0×10⁸ CAR⁺ T cells. In some examples, the first dose of the population of genetically engineered T cells is about 2.0×10⁸ CAR⁺ T cells. In some examples, the first dose of the population of genetically engineered T cells is about 3.0×10⁸ CAR⁺ T cells. In some examples, the first dose of the population of genetically engineered T cells is about 4.5×10⁸ CAR⁺ T cells. In some examples, the first dose of the population of genetically engineered T cells is about 6.0×10⁸ CAR⁺ T cells. In some examples, the human patient received no more than 7×10⁴ T cell receptor-positive (TCR⁺) cells/kg in each dose of the genetically engineered T cells.

In some embodiments, the lymphodepletion treatment in step (i) comprises co-administration to the human patient fludarabine at about 30 mg/m² and cyclophosphamide at about 500-750 mg/m² per day for three days. In some embodiments, the lymphodepletion treatment is performed about 2-7 days prior to administration of the genetically engineered T cells.

In some embodiments, the human patient does not show one or more of the following features prior to the lymphodepletion treatment: (a) significant worsening of clinical status, (b) requirement for supplemental oxygen to maintain a saturation level of greater than 91%, (c) uncontrolled cardiac arrhythmia, (d) hypotension requiring vasopressor support, (e) active infection, and (f) grade ≥2 acute neurological toxicity.

In some embodiments, after the lymphodepletion treatment step (i) and prior to the administration of the genetically engineered T cells of step (ii), the human patient does not show one or more of the following features: (a) active uncontrolled infection; (c) worsening of clinical status compared to the clinical status prior to step (i); and (c) neurological toxicity known to increase risk of immune effector cell-associated neurotoxicity syndrome (ICANS).

Any of the methods disclosed herein may further comprise (iii) administering to the human patient one or more subsequent doses of the population of genetically engineered T cells. Each of the subsequent doses of the genetically engineered T cells may be preceded by an LD treatment (e.g., following the LD treatment conditions and timing as disclosed herein). In some embodiments, the human patient achieves a partial response (PR) or a complete response (CR) to the first dose of the population of genetically engineered T cells. The one or more of the subsequent doses of the genetically engineered T cells may range from about 3×10⁷ to about 6×10⁸ CAR⁺ T cells. In some instances, the subsequent dose(s) may be identical to the first dose. In other instances, the subsequent dose(s) may be higher than the first dose. Alternatively, the subsequent dose(s) may be lower than the first dose.

In some embodiments, the human patient may meet one or more of the following criteria prior to receiving the subsequent dose(s): (a) confirmation of CD19+ tumor at relapse; (b) no prior art dose-limiting-toxicity (DLT); (c) no prior grade ≥3 cytokine release syndrome (CRS) without resolution to grade ≤2 within 72 hours following the first dose; (d) no prior graft-versus-host disease (GvHD) following the first dose; and (e) no prior grade 4 ICANS following the first dose.

Any of the methods disclosed herein may further comprise: (iv) monitoring the human patient for development of acute toxicity after administration of the population of genetically engineered T cells; and (v) managing the acute toxicity when occurs. In some instances, the acute toxicity comprises tumor lysis syndrome (TLS), cytokine release syndrome (CRS), neurotoxicity, B cell aplasia, hemophagocytic lymphohistiocytosis (HLH), cytopenia, graft-versus-host disease (GvHD), hypertension, renal insufficiency, or a combination thereof. In some examples, neurotoxicity may comprise ICANS, viral encephalitis, or a combination thereof, The human patient for treatment by the methods disclosed herein may have a relapsed and/or refractory B-cell malignancy. Exemplary B-cell malignancies (e.g., relapsed and/or refractory B-cell malignancies) include follicular lymphoma (FL), which optionally is grade 1-3a, mantle cell lymphoma (MCL), marginal zone lymphoma (MZL), chronic lymphocytic leukemia (CLL) and/or small lymphocytic lymphoma (SLL), and a large B cell lymphoma (LBLC). In some examples, FL is a grade 1-3a FL. In some examples, the LBLC is diffuse large B cell lymphoma (DLBCL), e.g., DLBCL not otherwise specified (NOS), high-grade B cell lymphoma with MYC and BCL2 and/or BCL6 rearrangements, primary mediastinal large B cell lymphoma (PMBCL), transformed FL and grade 3b FL, transformed MCL, or transformed MZL.

The human patient for treatment by the methods disclosed herein may have undergone one or more prior anti-cancer therapies. In one example, the human patient has grade 1-3a FL and has progressed after two lines of systemic therapy or has early relapse, wherein the systemic therapy optionally comprises an anti-CD20 antibody. In another example, the human patient has MZL and has relapsed and/or refractory disease after up to 5 prior lines of therapy, which comprise at least an anti-CD20 antibody. In yet another example, the human patient has MCL and has relapsed and/or refractory disease after up to 5 prior lines of therapy, which comprise at least anthracycline- or bendamustine-containing chemotherapy, an anti-CD20 antibody, or a Bruton tyrosine kinase (BTK) inhibitor (e.g., ibrutinib). In still another example, the human patient has CLL/SLL and progressed after at least 2 prior therapies comprising a BTK inhibitor (e.g., ibrutinib) and venetoclax. In another example, the human patient has a high-grade LBCL and has 2 or more lines of prior therapy comprising an anti-CD20 antibody and an anthracycline-containing chemotherapy. The high-grade LBCL may be DLBCL NOS, high-grade B cell lymphoma with MYC and BCL2 and/or BCL6 rearrangements, grade3b FL, transformed FL, transformed MCL, transformed MZL, or PMBCL. In one specific example, the human patient has transformed FL and has received at least one line of chemotherapy after transformation to DLBCL. In yet another example, the human patient has DLBCL and has received a prior CD-19-directed autologous CAR-T cell therapy.

In any of the methods disclosed herein, the anti-CD19 scFv comprises the V_{H} Comprising the amino acid sequence of SEQ ID NO: 81 and the V_{L} comprising the amino acid sequence of SEQ ID NO: 82. Such an anti-CD19 scFv may comprise the amino acid sequence of SEQ ID NO: 77. In some examples, the anti-CD19 CAR comprises the amino acid sequence of SEQ ID NO: 74. In some examples, a fragment comprising the nucleotide sequence of SEQ ID NO: 18 in the TRAC gene is deleted and replaced by the nucleic acid encoding the anti-CD19 CAR. In one specific example, the disrupted TRAC gene comprises the nucleotide sequence of SEQ ID NO: 90.

In any of the methods disclosed herein, the disrupted β2M gene in the T cells comprises one or more of the nucleotide sequences listed in Table 2. Alternatively or in addition, the disrupted Reg1 gene in the T cells comprises one or more of the nucleotide sequences listed in Table 4. Alternatively or in addition, the disrupted TGFBRII gene in the T cells comprises one or more of the nucleotide sequences listed in Table 3.

In some embodiments, at least 30% of the T cells in the population express the anti-CD19 CAR, wherein at least 90% of the T cells in the population are TCR−, at least 60% of the T cells in the population are β2M−, at least 80% of the T cells in the population are TGFBRII−, and/or at least 90% of the T cells in the population are Reg1−. In some examples, the population of genetically engineered T cells comprises: (a) at least 50% of the T cells express the anti-CD19 CAR; (b) at least 99% of the T cells are TCR−; (c) about 65% to about 80% of the T cells are β2M−; (d) about 80% to about 90% of the T cells are TGFBRII−; and/or (e) about 95% to about 97% of the T cells are Reg1−. Any of the population of genetically engineered T cells disclosed herein may be suspected in a solution comprising human serum albumin and a cryopreservative solution.

In some embodiments, the population of genetically engineered T cells comprise human primary T cells. Such T cells may be derived from one or more healthy human donors. In some embodiments, the population of genetically engineered T cells is allogeneic to the human patient.

Also provided herein are pharmaceutical compositions comprising any of the genetically engineered anti-CD19 CAR-T cells disclosed herein (e.g., CTX112 cells) for use in treating any of the B-cell malignancies as also disclosed herein or for use in manufacturing a medicament for the intended therapeutic purposes.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

FIGS. 1A-1C illustrate that TGFBRII disruption eliminates TGF-β inhibition on cell expansion by. FIG. 1A: inhibition of the growth of mock-electroporated human T cells by TGF-β. FIG. 1B: elimination of TGF-β inhibition of the growth of T cells edited to lack TGFBRII (along with disruptions in the TRAC, β2M, and Regnase-1 loci) without CAR. FIG. 1C: elimination of TGF-β inhibition of the growth of T cells edited to knock out TGFBRII (along with disruptions in the TRAC, B2M, and Regnase-1 loci) and expressing an anti-CD19 CAR [CAR-T (R/T)], R/T referring to disruption of Reg1 and TGFBRII.

FIGS. 2A-2E illustrate that the anti-CD19 CAR+TRAC KO+β2M KO+R/T KO T cells selectively secrete IFN-γ in the presence of CD19 expression. FIG. 2A: no high-level secretion of IFN-γ in the presence of the CD19-negative K562 cell line. FIG. 2B: secretion of high levels of IFN-7 when CD19 was expressed in K562 cells (K562-CD19). FIG. 2C: secretion of high levels of IFN-γ in human CD19-positive Raji-luciferase lymphoma cells. FIG. 2D: secretion of high levels of IFN-γ in human CD19-positive Nalm6-leukemia cells. FIG. 2E: secretion of high levels of IFN-γ in human CD19-positive Jeko-1 lymphoma cells. CAR-T (R/T) refers to anti-CD19 CAR-T cells with disrupted TRAC, B2M, Reg1, and TGFBRII.

FIGS. 3A-3E illustrate that the anti-CD19 CAR+TRAC KO+β2M KO+R/T KO T cells specifically kill CD19-expressing cells with high levels of cytotoxicity. FIG. 3A: levels of cytotoxic activity against the CD19-negative cell line K562 are not above control cells—mock (TCR⁺ T cells that were mock electroporated) or no CAR (cells containing TRAC, β2M, and R/T edits except for CAR insertion). FIG. 3B: high levels of cytotoxicity in K562 cells engineered to express human CD19 (K562-CD19). FIG. 3C: high levels of cytotoxicity in human CD19-positive Raji-luciferase lymphoma cells. FIG. 3D: high levels of cytotoxicity in human CD19-positive Nalm6-leukemia cells. FIG. 3E: high levels of cytotoxicity in human CD19-positive Jeko-1 lymphoma cells. CAR-T (R/T) refers to anti-CD19 CAR-T cells with disrupted TRAC, B2M, Reg1, and TGFBRII.

FIGS. 4A-4D illustrate that R/T disruptions synergistically increase potency of the anti-CD19 CAR+TRAC KO+β2M KO+R/T KO T cells against CD19-positive malignancies. FIG. 4A: increased survival (probability of survival) of CD19⁺ Nalm6 leukemia mice. FIG. 4B: increased survival (probability of survival) of CD19⁺ Jeko-1 lymphoma mice. FIG. 4C: increased expansion and persistence (CAR copies per g of DNA) of the anti-CD19 CAR+TRAC KO+β2M KO+R/T KO T cells relative to the anti-CD19 CAR+TRAC KO+β2M KO T cells or the anti-CD19 CAR+TRAC KO+β2M KO+TGFBR2 KO T cells or the anti-CD19 CAR+TRAC KO+β2M KO+Regnase-1 KO T cells in Nalm6 models. FIG. 4D: increased expansion and persistence (CAR copies per g of DNA) of the anti-CD19 CAR+TRAC KO+β2M KO+R/T KO T cells relative to the anti-CD19 CAR+TRAC KO+β2M KO T cells or the anti-CD19 CAR+TRAC KO+β2M KO+TGFBR2 KO T cells or the anti-CD19 CAR+TRAC KO+β2M KO+Regnase-1 KO T cells in Jeko-1 models. CAR-T refers to anti-CD19 CAR-T cells with disrupted TRAC and B2M. CAR-T (R/T) refers to anti-CD19 CAR-T cells with disrupted TRAC, B2M, Reg1, and TGFBRII.

FIGS. 5A-5B illustrate that polyclonal anti-CD19 CAR T cells with TRAC, β2M, Reg1 and TGFBRII knockouts persist in both female (FIG. 5A) and male (FIG. 5B) mice.

FIGS. 6A-6C illustrate that anti-CD19 CAR+TRAC KO+β2M KO+Reg1 KO+TGFBRII KO T cells control leukemia/lymphoma at low cell doses. FIG. 6A: high level of tumor control in Raji-luciferase lymphoma cells. FIG. 6B: high level of tumor control in Nalm6-leukemia cells. FIG. 6C: high level of tumor control in Jeko-1 lymphoma model at doses lower than a sub-efficacious dose of anti-CD19 CAR T cells with only TRAC and β2M knockouts. BLI: bioluminescence imaging.

FIGS. 7A-7B illustrate that anti-CD19 CAR T cells with TRAC, β2M, Reg1 and TGFBRII knockouts (FIG. 7A) are efficacious in treating Nalm6-leukemia mice at lower doses relative to anti-CD19 CAR T cells with only TRAC and, β2M knockouts (FIG. 7B).

FIGS. 8A-8B include diagrams showing target specificity and cytokine-dependent growth of anti-CD19 CAR T cells with TRAC, β2M, Reg1, and TGFBRII disruptions (e.g., CTX112 cells). FIG. 8A: a chart showing that both CTX112 cells and counterpart CAR-T cells (with no disruptions in TGFBRII and Regnase-1 genes) specifically target CD19+ cells, but not other tissue cells lacking CD19 expression. FIG. 8B: a diagram showing that CTX112 cells require cytokines for cell growth.

FIG. 9 illustrates GLP-compliant tumorigenicity study in NSG mice using anti-CD19 CAR T cells with TRAC, β2M, Reg1, and TGFBRII knockouts. GLP: Good Laboratory Practice; RT: radiation therapy. CAR-T (R/T) refers to anti-CD19 CAR-T cells with disrupted TRAC, B2M, Reg1, and TGFBRII.

FIG. 10 illustrates body weight change in mice treated with both low dose (0.5×10⁶ cells/mouse) and high dose (1×10⁷ cells/mouse) of anti-CD19 CAR T cells with TRAC, β2M, Reg1, and TGFBRII knockouts. CAR-T (R/T) refers to anti-CD19 CAR-T cells with disrupted TRAC, B2M, Reg1, and TGFBRII.

FIG. 11 illustrates exposure of anti-CD19 CAR T cells with TRAC, β2M, Reg1, and TGFBRII knockouts in mouse blood. CAR-T (R/T) refers to anti-CD19 CAR-T cells with disrupted TRAC, B2M, Reg1, and TGFBRII.

FIG. 12 illustrates that anti-CD19 CAR T cells with TRAC and β2M knockouts elicit comparable allogeneic mixed lymphocyte reaction (MLR) responses in vitro with or without further Reg1 and TGFBRII knockouts. CAR-T refers to anti-CD19 CAR-T cells with disrupted TRAC and B2M. CAR-T (R/T) refers to anti-CD19 CAR-T cells with disrupted TRAC, B2M, Reg1, and TGFBRII.

FIGS. 13A-13B illustrate that allogeneic natural killer (NK) cells from both donor 1 (FIG. 13A) and donor 2 (FIG. 13B) can lyse anti-CD19 CAR T cells with only TRAC and, β2M knockouts or anti-CD19 CAR T cells with TRAC, β2M, Reg1 and TGFBRII knockouts in vitro. CAR-T refers to anti-CD19 CAR-T cells with disrupted TRAC and B2M. CAR-T (R/T) refers to anti-CD19 CAR-T cells with disrupted TRAC, B2M, Reg1, and TGFBRII.

FIGS. 14A-14C illustrate comparable response to allogeneic NK and T cell from donor 1 (FIG. 14A), donor 2 (FIG. 14B) and donor 3 (FIG. 14C) in vivo elicited by anti-CD19 CAR T cells with only TRAC and β2M knockouts or anti-CD19 CAR T cells with TRAC, β2M, Reg1 and TGFBRII knockouts. CAR-T refers to anti-CD19 CAR-T cells with disrupted TRAC and B2M. CAR-T (R/T) refers to anti-CD19 CAR-T cells with disrupted TRAC, B2M, Reg1, and TGFBRII.

FIG. 15 is a diagram illustrating an exemplary study schema for the clinical studies described in Example 12 below. DLT: dose-limiting toxicity; LD: lymphodepleting. LD chemotherapy consists of co-administration of fludarabine 30 mg/m² and cyclophosphamide 500 mg/m² IV daily for 3 days.

FIG. 16 is a diagram illustrating an exemplary study design for the clinical studies described in Example 12 below, involving dose optimization in patients having specific diseases as indicated. CLL: chronic lymphocytic leukemia; DLBCL: diffuse large B cell lymphoma FL: follicular lymphoma; MCL: mantle cell lymphoma; MZL: marginal zone lymphoma: SLL: small lymphocytic lymphoma.

### DETAILED DESCRIPTION OF THE INVENTION

The present disclosure aims at developing effective methods for treating relapsed and/or refractory B-cell malignancies using genetically engineered anti-CD19 CAR-T cells having improved growth activity, persistence, reduced T cell exhaustion, and enhanced potency, a long-felt need in CAR-T therapy. The anti-CD19 CAR-T cells disclosed herein may comprise multiple genetic edits on endogenous genes, for example, disruption of the TRAC gene, the β2M gene, the TGFBRII gene, and the Reg1 gene, to make the cells suitable for allogeneic immune cell therapy and to achieve features that could improve treatment efficacy. For example, β2M disruption can reduce the risk of or prevent a host-versus-graft response and TRAC disruption can reduce the risk of or prevent a graft-versus-host response. In addition, TGFBRII disruption may reduce immunosuppressive effect of transforming growth factor beta (TGF-β) in the tumor microenvironment and Reg1 disruption may improve CAR-T cell functionality via long-term persistence with robust effector function.

Such a T cell may use bona fide T cells as the starting material, for example, non-transformed T cells, terminally differentiated T cells, T cells having stable genome, and/or T cells that depend on cytokines and growth factors for proliferation and expansion. Alternatively, such a T cell may use T cells generated from precursor cells such as hematopoietic stem cells (e.g., iPSCs), e.g., in vitro culture. The T cells disclosed herein may confer one or more benefits in both CAR-T cell manufacturing and clinical applications.

Unexpectedly, the present disclosure reports that disruptions of Reg1 and TGFBRII synergistically acted to increase expansion and functional persistence of the anti-CD19 CAR-T cells and enhanced therapeutic efficacy against CD19+ cancers as observed in animal models. The anti-CD19 CAR-T cell disclosed herein, having both Reg1 and TGFBRII genes knocked out, exhibited effective anti-tumor activities at low doses relative to anti-CD19 CAR-T cells with intact Reg1 and TGFBRII and long-term-persistence in vivo with robust CAR-T cell functionality. Further, it was observed that the anti-CD19 CAR-T cells disclosed here were effectively cleared in an immunocompetent mouse model.

Other advantageous features associated with the disruption of Reg1 gene and/or the TGFBRII gene may be found in WO 2022/064428, the relevant disclosures of which are incorporated by reference for the subject matter and purpose referenced herein.

Accordingly, provided herein are methods for treating B-cell malignancies such as relapsed and/or refractory B cell malignancies (e.g., those disclosed herein) using anti-CD19 CAR-T cells having improved persistence and enhanced anti-tumor activity, methods of producing such T cells, and therapeutic applications of such T cells in eliminating CD19+ disease cells such as cancer cells.

### I. Anti-CD19 CAR-T Cells Having Enhanced Features

In some aspects, provided herein are anti-CD19 CAR-T cells with multiple genetic modifications to improve CAR-T cell functionality and thus therapeutic efficacy. The genetically engineered T cells provided herein express a chimeric antigen receptor (CAR) that binds CD19 and have multiple genetic edits on endogenous genes, for example, on a TRAC gene, a β2M gene, a Reg1 gene, and a TGFBRII gene.

The genetically engineered T cells may be derived from parent T cells (e.g., non-edited wild-type T cells) obtained from a suitable source, for example, one or more mammal donors. In some examples, the parent T cells are primary T cells (e.g., non-transformed and terminally differentiated T cells) obtained from one or more human donors. Alternatively, the parent T cells may be differentiated from precursor T cells obtained from one or more suitable donor or stem cells such as hematopoietic stem cells or inducible pluripotent stem cells (iPSC), which may be cultured in vitro.

Any of the genetically engineered T cells may be generated via gene editing (including genomic editing), a type of genetic engineering in which nucleotide(s)/nucleic acid(s) is/are inserted, deleted, and/or substituted in a DNA sequence, such as in the genome of a targeted cell. Targeted gene editing enables insertion, deletion, and/or substitution at pre-selected sites in the genome of a targeted cell (e.g., in a targeted gene or targeted DNA sequence). When a sequence of an endogenous gene is edited, for example by deletion, insertion or substitution of nucleotide(s)/nucleic acid(s), the endogenous gene comprising the affected sequence may be knocked-out due to the sequence alteration. Therefore, targeted editing may be used to disrupt endogenous gene expression. “Targeted integration” refers to a process involving insertion of one or more exogenous sequences, with or without deletion of an endogenous sequence at the insertion site. Targeted integration can result from targeted gene editing when a donor template containing an exogenous sequence is present.

A. Genetically Edited Genes

In some aspects, the present disclosure provides genetically engineered T cells that may comprise a disrupted Reg1 gene, a disrupted TGFBRII gene, a disrupted TRAC gene, and/or a disrupted β2M gene.

As used herein, a “disrupted gene” refers to a gene comprising an insertion, deletion or substitution relative to an endogenous gene such that expression of a functional protein from the endogenous gene is reduced or inhibited. As used herein, “disrupting a gene” refers to a method of inserting, deleting or substituting at least one nucleotide/nucleic acid in an endogenous gene such that expression of a functional protein from the endogenous gene is reduced or inhibited. Methods of disrupting a gene are known to those of skill in the art and described herein.

In some embodiments, a cell that comprises a disrupted gene does not express (e.g., at the cell surface) a detectable level (e.g., in an immune assay using an antibody binding to the encoded protein or by flow cytometry) of the protein encoded by the gene. A cell that does not express a detectable level of the protein may be referred to as a knockout cell.

Reg1 Gene Editing

In some embodiments, the genetically engineered T cells may comprise a disrupted gene involved in mRNA decay. Such a gene may be Reg1. Reg1 contains a zinc finger motif, binds RNA and exhibits ribonuclease activity. Reg1 plays roles in both immune and non-immune cells and its expression can be rapidly induced under diverse conditions including microbial infections, treatment with inflammatory cytokines and chemical or mechanical stimulation. Human Reg1 gene is located on chromosome 1p34.3. Additional information can be found in GenBank under Gene ID: 80149.

In some examples, the genetically engineered T cells may comprise a disrupted Reg1 gene such that the expression of Reg1 in the T cells is substantially reduced or eliminated completely. The disrupted Reg1 gene may comprise one or more genetic edits at one or more suitable target sites (e.g., in coding regions or in non-coding regulatory regions such as promoter regions) that disrupt expression of the Reg1 gene. Such target sites may be identified based on the gene editing approach for use in making the genetically engineered T cells. Exemplary target sites for the genetic edits may include exon 1, exon 2, exon 3, exon 4, exon 5, exon 6, or a combination thereof. In some examples, one or more genetic editing may occur in exon 2 or exon 4. Such genetic editing may be induced by the CRISPR/Cas technology using a suitable guide RNA, for example, those listed in Table 1. The resultant edited Reg1 gene using a gRNA listed in Table 1 may comprise one or more edited sequences provided in Table 4 below.

Disruption of the Reg1 gene can enhance long-term-persistence and maintain robust effector function, thereby improving T cell functionality.

TGFBRII Gene Editing

In some embodiments, the genetically engineered T cells may comprise a disrupted TGFBRII gene, which encodes Transforming Growth Factor Receptor Type II (TGFBRII). TGFBRII receptors are a family of serine/threonine kinase receptors involved in the TGFβ signaling pathway. These receptors bind growth factor and cytokine signaling proteins in the TGFβ family, for example, TGFβs (TGFβ1, TGFβ2, and TGFβ3), bone morphogenetic proteins (BMPs), growth differentiation factors (GDFs), activin and inhibin, myostatin, anti-Müllerian hormone (AMH), and NODAL.

In some examples, the genetically engineered T cells may comprise a disrupted TGFBRII gene such that the expression of TGFBRII in the T cells is substantially reduced or eliminated completely. The disrupted TGFBRII gene may comprise one or more genetic edits at one or more suitable target sites (e.g., in coding regions or in non-coding regulatory regions such as promoter regions) that disrupt expression of the TGFBRII gene. Such target sites may be identified based on the gene editing approach for use in making the genetically engineered T cells. Exemplary target sites for the genetic edits may include exon 1, exon 2, exon 3, exon 4, exon 5, or a combination thereof. In some examples, one or more genetic editing may occur in exon 4 and/or exon 5. Such genetic editing may be induced by a gene editing technology, (e.g., the CRISPR/Cas technology) using a suitable guide RNA, for example, those listed in Table 1. The resultant edited TGFBRII gene using a gRNA listed in Table 1 may comprise one or more edited sequences provided in Table 3 below.

Disruption of the TGFBRII gene can eliminate surface expression of TGFBRII and reduce the immunosuppressive effect of transforming growth factor beta (TFG-β) in the tumor microenvironment.

β2M Gene Edit

In some embodiments, the genetically engineered T cells disclosed herein may further comprise a disrupted β2M gene. β2M is a common (invariant) component of MHC I complexes. Disrupting its expression by gene editing will prevent host versus therapeutic allogeneic T cells responses leading to increased allogeneic T cell persistence. In some embodiments, expression of the endogenous β2M gene is eliminated to prevent a host-versus-graft response.

In some embodiments, an edited β2M gene may comprise a nucleotide sequence selected from the following sequences in Table 1. It is known to those skilled in the art that different nucleotide sequences in an edited gene such as an edited β2M gene (e.g., those in Table 2) may be generated by a single gRNA such as the one listed in Table 1 (β2M−1). See also WO2019097305, the relevant disclosures of which are incorporated by reference for the subject matter and purpose referenced herein.

TRAC Gene Edit

In some embodiments, the genetically engineered T cells as disclosed herein may further comprise a disrupted TRAC gene. This disruption leads to loss of function of the TCR and renders the engineered T cell non-alloreactive and suitable for allogeneic transplantation, minimizing the risk of graft versus host disease. In some embodiments, expression of the endogenous TRAC gene is eliminated to prevent a graft-versus-host response. See also WO2019097305, the relevant disclosures of which are incorporated by reference herein for the purpose and subject matter referenced herein.

Such genetic editing of the TRAC gene may be induced by a gene editing technology, (e.g., the CRISPR/Cas technology) using a suitable guide RNA, for example, those listed in Table 1. It should be understood that more than one suitable target site/gRNA can be used for each target gene disclosed herein, for example, those known in the art or disclosed herein. Additional examples can be found in, e.g., WO2019097305, the relevant disclosures of which are incorporated by reference herein for the purpose and subject matter referenced herein.

In some instances, a nucleic acid encoding an anti-CD19 CAR may be inserted into the TRAC gene, thereby disrupting expression of the TRAC gene. For example, the CAR-coding nucleic acid may replace the target site of a gRNA used in gene editing via CRISPR/Cas9 (e.g., replacing the fragment comprising SEQ ID NO: 18 in the TRAC gene.

B. Anti-CD19 Chimeric Antigen Receptor (CAR)

A chimeric antigen receptor (CAR) refers to an artificial immune cell receptor that is engineered to recognize and bind to an antigen expressed by undesired cells, for example, disease cells such as cancer cells. A T cell that expresses a CAR polypeptide is referred to as a CAR T cell. CARs have the ability to redirect T-cell specificity and reactivity toward a selected target in a non-MHC-restricted manner. The non-MHC-restricted antigen recognition gives CAR-T cells the ability to recognize an antigen independent of antigen processing, thus bypassing a major mechanism of tumor escape. Moreover, when expressed on T-cells, CARs advantageously do not dimerize with endogenous T-cell receptor (TCR) alpha and beta chains.

There are various generations of CARs, each of which contains different components. First generation CARs join an antibody-derived scFv to the CD3zeta ((or z) intracellular signaling domain of the T-cell receptor through hinge and transmembrane domains. Second generation CARs incorporate an additional co-stimulatory domain, e.g., CD28, 4-1BB (41BB), or ICOS, to supply a costimulatory signal. Third-generation CARs contain two costimulatory domains (e.g., a combination of CD27, CD28, 4-1BB, ICOS, or OX40) fused with the TCR CD3ζ chain. Maude et al., Blood. 2015; 125(26):4017-4023; Kakarla and Gottschalk, Cancer J. 2014; 20(2):151-155). Any of the various generations of CAR constructs is within the scope of the present disclosure.

Generally, a CAR is a fusion polypeptide comprising an extracellular domain that recognizes a target antigen (e.g., a single chain fragment (scFv) of an antibody or other antibody fragment) and an intracellular domain comprising a signaling domain of the T-cell receptor (TCR) complex (e.g., CD3ζ) and, in most cases, a co-stimulatory domain. (Enblad et al., Human Gene Therapy. 2015; 26(8):498-505). A CAR construct may further comprise a hinge and transmembrane domain between the extracellular domain and the intracellular domain, as well as a signal peptide at the N-terminus for surface expression. Examples of signal peptides include SEQ ID NO: 51 and SEQ ID NO: 52 as provided in Table 5 below. Other signal peptides may be used.

(i) Antigen Binding Extracellular Domain

The antigen-binding extracellular domain is the region of a CAR polypeptide that is exposed to the extracellular fluid when the CAR is expressed on cell surface. In some instances, a signal peptide may be located at the N-terminus to facilitate cell surface expression. In some embodiments, the antigen binding domain can be a single-chain variable fragment (scFv, which may include an antibody heavy chain variable region (V_{H}) and an antibody light chain variable region (V_{L}) (in either orientation). In some instances, the V_{H} and V_{L} fragment may be linked via a peptide linker. The linker, in some embodiments, includes hydrophilic residues with stretches of glycine and serine for flexibility as well as stretches of glutamate and lysine for added solubility. The scFv fragment retains the antigen-binding specificity of the parent antibody, from which the scFv fragment is derived. In some embodiments, the scFv may comprise humanized V_{H} and/or V_{L} domains. In other embodiments, the V_{H} and/or V_{L} domains of the scFv are fully human.

The antigen-binding extracellular domain may be specific to a CD19 antigen, such as a human CD19 antigen.

In some embodiments, the antigen-binding extracellular domain can be a single-chain variable fragment (scFv) that binds CD19 as disclosed herein. The scFv may comprise an antibody heavy chain variable region (V_{H}) and an antibody light chain variable region (V_{L}), which optionally may be connected via a flexible peptide linker. In some instances, the scFv may have the V_{H} to V_{L} orientation (from N-terminus to C-terminus). Alternatively the scFv may have the V_{L} to V_{H} orientation (from N-terminus to C-terminus).

In some examples, the antigen-binding extracellular domain can be a single-chain variable fragment (scFv) that binds human CD19. In some instances, the anti-CD19 scFv may comprises (i) a heavy chain variable region (V_{H}) that comprises the same heavy chain complementary determining regions (CDRs) as those in SEQ ID NO: 81; and (ii) a light chain variable region (V_{L}) that comprises the same light chain CDRs as those in SEQ ID NO: 82. See Table 5 below. In some specific examples, the anti-CD19 antibody discloses herein may comprise the heavy chain CDR1, heavy chain CDR2, and heavy chain CDR3 set forth as SEQ ID NOs: 63-65, respectively as determined by the Kabat method. Alternatively or in addition, the anti-CD19 antibody discloses herein may comprise the light chain CDR1, light chain CDR2, and light chain CDR3 set forth as SEQ ID NOs:60-62 as determined by the Kabat method. Alternatively, the anti-CD19 antibody discloses herein may comprise the heavy chain CDR1, heavy chain CDR2, and heavy chain CDR3 set forth as SEQ ID NOs: 69-71, respectively as determined by the Chothia method. Alternatively or in addition, the anti-CD19 antibody discloses herein may comprise the light chain CDR1, light chain CDR2, and light chain CDR3 set forth as SEQ ID NOs:66-68 as determined by the Chothia method. In one specific example, the anti-CD19 scFv may comprise a V_{H} Comprising the amino acid sequence of SEQ ID NO: 81 and a V_{L} comprises the amino acid sequence of SEQ ID NO: 82. See Sequence Table 5 below.

Two antibodies having the same V_{H} and/or V_{L} CDRs means that their CDRs are identical when determined by the same approach (e.g., the Kabat approach, the Chothia approach, the AbM approach, the Contact approach, or the IMGT approach as known in the art. See, e.g., bioinf.org.uk/abs/or abysis.org/abysis/sequence_input).

(ii) Transmembrane Domain

The anti-CD19 CAR polypeptide disclosed herein may contain a transmembrane domain, which can be a hydrophobic alpha helix that spans the membrane. As used herein, a “transmembrane domain” refers to any protein structure that is thermodynamically stable in a cell membrane, preferably a eukaryotic cell membrane. The transmembrane domain can provide stability of the anti-CD19 CAR containing such.

In some embodiments, the transmembrane domain of a anti-CD19 CAR as provided herein can be a CD8 transmembrane domain. In other embodiments, the transmembrane domain can be a CD28 transmembrane domain. In yet other embodiments, the transmembrane domain is a chimera of a CD8 and CD28 transmembrane domain. Other transmembrane domains may be used as provided herein. In some embodiments, the transmembrane domain is a CD8a transmembrane domain containing the sequence of SEQ ID NO: 53 as provided below in Table 5. Other transmembrane domains may be used.

(iii) Hinge Domain

In some embodiments, a hinge domain may be located between an extracellular domain (comprising the antigen binding domain) and a transmembrane domain of a anti-CD19 CAR, or between a cytoplasmic domain and a transmembrane domain of the anti-CD19 CAR. A hinge domain can be any oligopeptide or polypeptide that functions to link the transmembrane domain to the extracellular domain and/or the cytoplasmic domain in the polypeptide chain. A hinge domain may function to provide flexibility to the anti-CD19 CAR, or domains thereof, or to prevent steric hindrance of the anti-CD19 CAR, or domains thereof.

In some embodiments, a hinge domain may comprise up to 300 amino acids (e.g., 10 to 100 amino acids, or 5 to 20 amino acids). In some embodiments, one or more hinge domain(s) may be included in other regions of a anti-CD19 CAR. In some embodiments, the hinge domain may be a CD8 hinge domain. Other hinge domains may be used.

(iv) Intracellular Signaling Domains

Any of the anti-CD19 CAR constructs contain one or more intracellular signaling domains (e.g., CD3ζ, and optionally one or more co-stimulatory domains), which are the functional end of the receptor. Following antigen recognition, receptors cluster and a signal is transmitted to the cell.

CD3ζ is the cytoplasmic signaling domain of the T cell receptor complex. CD3ζ contains three (3) immunoreceptor tyrosine-based activation motif (ITAM)s, which transmit an activation signal to the T cell after the T cell is engaged with a cognate antigen. In many cases, CD3ζ provides a primary T cell activation signal but not a fully competent activation signal, which requires a co-stimulatory signaling.

In some embodiments, the anti-CD19 CAR polypeptides disclosed herein may further comprise one or more co-stimulatory signaling domains. For example, the co-stimulatory domains of CD28 and/or 4-1BB may be used to transmit a full proliferative/survival signal, together with the primary signaling mediated by CD3ζ. In some examples, the CAR disclosed herein comprises a CD28 co-stimulatory molecule. In other examples, the CAR disclosed herein comprises a 4-1BB co-stimulatory molecule. In some embodiments, a CAR includes a CD3ζ signaling domain and a CD28 co-stimulatory domain. In other embodiments, a CAR includes a CD3ζ signaling domain and 4-1BB co-stimulatory domain. In still other embodiments, a CAR includes a CD3ζ signaling domain, a CD28 co-stimulatory domain, and a 4-1BB co-stimulatory domain.

Table 5 provides examples of signaling domains derived from 4-1BB, CD28 and CD3-zeta that may be used herein.

In specific examples, the anti-CD19 CAR disclosed herein may comprise the amino acid sequence of SEQ ID NO: 73, which may be encoded by the nucleotide sequence of SEQ ID NO: 72. Alternatively, the anti-CD19 CAR may be a mature form without the N-terminal signal peptide, e.g., comprising the amino acid sequence of SEQ ID NO:74.

C. Methods of Making Genetically Engineered T Cells

The genetically engineered T cells disclosed herein can be prepared by genetic editing of parent T cells or precursor cells thereof via a conventional gene editing method or those described herein.

(a) T Cells

In some embodiments, T cells can be derived from one or more suitable mammals, for example, one or more human donors. T cells can be obtained from a number of sources, including, but not limited to, peripheral blood mononuclear cells, bone marrow, lymph nodes tissue, cord blood, thymus issue, tissue from a site of infection, ascites, pleural effusion, spleen tissue, and tumors. In certain embodiments, T cells can be obtained from a unit of blood collected from a subject using any number of techniques known to the skilled person, such as sedimentation, e.g., FICOLL™ separation.

In some examples, T cells can be isolated from a mixture of immune cells (e.g., those described herein) to produce an isolated T cell population. For example, after isolation of peripheral blood mononuclear cells (PBMC), both cytotoxic and helper T lymphocytes can be sorted into naive, memory, and effector T cell subpopulations either before or after activation, expansion, and/or genetic modification.

A specific subpopulation of T cells, expressing one or more of the following cell surface markers: TCRαβ, CD3, CD4, CD8, CD27 CD28, CD38 CD45RA, CD45RO, CD62L, CD127, CD122, CD95, CD197, CCR7, KLRG1, MCH-I proteins and/or MCH-II proteins, can be further isolated by positive or negative selection techniques. In some embodiments, a specific subpopulation of T cells, expressing one or more of the markers selected from the group consisting of TCRab, CD4 and/or CD8, is further isolated by positive or negative selection techniques. In some embodiments, subpopulations of T cells may be isolated by positive or negative selection prior to genetic engineering and/or post genetic engineering.

An isolated population of T cells may express one or more of the T cell markers, including, but not limited to a CD3+, CD4+, CD8+, or a combination thereof. In some embodiments, the T cells are isolated from a donor, or subject, and first activated and stimulated to proliferate in vitro prior to undergoing gene editing.

In some instances, the T cell population comprises primary T cells isolated from one or more human donors. Such T cells are terminally differentiated, not transformed, depend on cytokines and/or growth factors for growth, and/or have stable genomes.

Alternatively, the T cells may be derived from stem cells (e.g., HSCs or iPSCs) via in vitro differentiation.

T cells from a suitable source can be subjected to one or more rounds of stimulation, activation and/or expansion. T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041. In some embodiments, T cells can be activated and expanded for about 1 day to about 4 days, about 1 day to about 3 days, about 1 day to about 2 days, about 2 days to about 3 days, about 2 days to about 4 days, about 3 days to about 4 days, or about 1 day, about 2 days, about 3 days, or about 4 days prior to introduction of the genome editing compositions into the T cells.

In some embodiments, T cells are activated and expanded for about 4 hours, about 6 hours, about 12 hours, about 18 hours, about 24 hours, about 36 hours, about 48 hours, about 60 hours, or about 72 hours prior to introduction of the gene editing compositions into the T cells. In some embodiments, T cells are activated at the same time that genome editing compositions are introduced into the T cells. In some instances, the T cell population can be expanded and/or activated after the genetic editing as disclosed herein. T cell populations or isolated T cells generated by any of the gene editing methods described herein are also within the scope of the present disclosure.

(b) Gene Editing Methods

Any of the genetically engineered T cells can be prepared using conventional gene editing methods or those described herein to edit one or more of the target genes disclosed herein (targeted editing). Targeted editing can be achieved either through a nuclease-independent approach, or through a nuclease-dependent approach. In the nuclease-independent targeted editing approach, homologous recombination is guided by homologous sequences flanking an exogenous polynucleotide to be introduced into an endogenous sequence through the enzymatic machinery of the host cell. The exogenous polynucleotide may introduce deletions, insertions or replacement of nucleotides in the endogenous sequence.

Alternatively, the nuclease-dependent approach can achieve targeted editing with higher frequency through the specific introduction of double strand breaks (DSBs) by specific rare-cutting nucleases (e.g., endonucleases). Such nuclease-dependent targeted editing also utilizes DNA repair mechanisms, for example, non-homologous end joining (NHEJ), which occurs in response to DSBs. DNA repair by NHEJ often leads to random insertions or deletions (indels) of a small number of endogenous nucleotides. In contrast to NHEJ mediated repair, repair can also occur by a homology directed repair (HDR). When a donor template containing exogenous genetic material flanked by a pair of homology arms is present, the exogenous genetic material can be introduced into the genome by HDR, which results in targeted integration of the exogenous genetic material.

In some embodiments, gene disruption may occur by deletion of a genomic sequence using two guide RNAs. Methods of using CRISPR-Cas gene editing technology to create a genomic deletion in a cell (e.g., to knock out a gene in a cell) are known (Bauer D E et al. Vis. Exp. 2015; 95: e52118).

Available endonucleases capable of introducing specific and targeted DSBs include, but not limited to, zinc-finger nucleases (ZFN), transcription activator-like effector nucleases (TALEN), and RNA-guided CRISPR-Cas9 nuclease (CRISPR/Cas9; Clustered Regular Interspaced Short Palindromic Repeats Associated 9). Additionally, DICE (dual integrase cassette exchange) system utilizing phiC31 and Bxb1 integrases may also be used for targeted integration. Some exemplary approaches are disclosed in detail below.

CRISPR-Cas9 Gene Editing System

The CRISPR-Cas9 system is a naturally-occurring defense mechanism in prokaryotes that has been repurposed as an RNA-guided DNA-targeting platform used for gene editing. It relies on the DNA nuclease Cas9, and two noncoding RNAs, crisprRNA (crRNA) and trans-activating RNA (tracrRNA), to target the cleavage of DNA. CRISPR is an abbreviation for Clustered Regularly Interspaced Short Palindromic Repeats, a family of DNA sequences found in the genomes of bacteria and archaea that contain fragments of DNA (spacer DNA) with similarity to foreign DNA previously exposed to the cell, for example, by viruses that have infected or attacked the prokaryote. These fragments of DNA are used by the prokaryote to detect and destroy similar foreign DNA upon re-introduction, for example, from similar viruses during subsequent attacks. Transcription of the CRISPR locus results in the formation of an RNA molecule comprising the spacer sequence, which associates with and targets Cas (CRISPR-associated) proteins able to recognize and cut the foreign, exogenous DNA. Numerous types and classes of CRISPR/Cas systems have been described (see, e.g., Koonin et al., (2017) Curr Opin Microbiol 37:67-78).

crRNA drives sequence recognition and specificity of the CRISPR-Cas9 complex through Watson-Crick base pairing typically with a 20 nucleotide (nt) sequence in the target DNA. Changing the sequence of the 5′ 20nt in the crRNA allows targeting of the CRISPR-Cas9 complex to specific loci. The CRISPR-Cas9 complex only binds DNA sequences that contain a sequence match to the first 20 nt of the crRNA, if the target sequence is followed by a specific short DNA motif (with the sequence NGG) referred to as a protospacer adjacent motif (PAM).

tracrRNA hybridizes with the 3′ end of crRNA to form an RNA-duplex structure that is bound by the Cas9 endonuclease to form the catalytically active CRISPR-Cas9 complex, which can then cleave the target DNA.

Once the CRISPR-Cas9 complex is bound to DNA at a target site, two independent nuclease domains within the Cas9 enzyme each cleave one of the DNA strands upstream of the PAM site, leaving a double-strand break (DSB) where both strands of the DNA terminate in a base pair (a blunt end).

After binding of CRISPR-Cas9 complex to DNA at a specific target site and formation of the site-specific DSB, the next key step is repair of the DSB. Cells use two main DNA repair pathways to repair the DSB: non-homologous end joining (NHEJ) and homology-directed repair (HDR).

NHEJ is a robust repair mechanism that appears highly active in the majority of cell types, including non-dividing cells. NHEJ is error-prone and can often result in the removal or addition of between one and several hundred nucleotides at the site of the DSB, though such modifications are typically <20 nt. The resulting insertions and deletions (indels) can disrupt coding or noncoding regions of genes. Alternatively, HDR uses a long stretch of homologous donor DNA, provided endogenously or exogenously, to repair the DSB with high fidelity. HDR is active only in dividing cells, and occurs at a relatively low frequency in most cell types. In many embodiments of the present disclosure, NHEJ is utilized as the repair operant.

Endonuclease for use in CRISPR

In some embodiments, the Cas9 (CRISPR associated protein 9) endonuclease is used in a CRISPR method for making the genetically engineered T cells as disclosed herein. The Cas9 enzyme may be one from Streptococcus pyogenes, although other Cas9 homologs may also be used. It should be understood, that wild-type Cas9 may be used or modified versions of Cas9 may be used (e.g., evolved versions of Cas9, or Cas9 orthologues or variants), as provided herein. In some embodiments, Cas9 may be substituted with another RNA-guided endonuclease, such as Cpf1 (of a class II CRISPR/Cas system).

In some embodiments, the CRISPR/Cas system comprises components derived from a Type-I, Type-II, or Type-III system. Updated classification schemes for CRISPR/Cas loci define Class 1 and Class 2 CRISPR/Cas systems, having Types I to V or VI (Makarova et al., (2015) Nat Rev Microbiol, 13(11):722-36; Shmakov et al., (2015) Mol Cell, 60:385-397). Class 2 CRISPR/Cas systems have single protein effectors. Cas proteins of Types II, V, and VI are single-protein, RNA-guided endonucleases, herein called “Class 2 Cas nucleases.” Class 2 Cas nucleases include, for example, Cas9, Cpf1, C2c1, C2c2, and C2c3 proteins. The Cpf1 nuclease (Zetsche et al., (2015) Cell 163:1-13) is homologous to Cas9 and contains a RuvC-like nuclease domain.

In some embodiments, the Cas nuclease is from a Type-II CRISPR/Cas system (e.g., a Cas9 protein from a CRISPR/Cas9 system). In some embodiments, the Cas nuclease is from a Class 2 CRISPR/Cas system (a single-protein Cas nuclease such as a Cas9 protein or a Cpf1 protein). The Cas9 and Cpf1 family of proteins are enzymes with DNA endonuclease activity, and they can be directed to cleave a desired nucleic acid target by designing an appropriate guide RNA, as described further herein.

In some embodiments, a Cas nuclease may comprise more than one nuclease domain. For example, a Cas9 nuclease may comprise at least one RuvC-like nuclease domain (e.g., Cpf1) and at least one HNH-like nuclease domain (e.g., Cas9). In some embodiments, the Cas9 nuclease introduces a DSB in the target sequence. In some embodiments, the Cas9 nuclease is modified to contain only one functional nuclease domain. For example, the Cas9 nuclease is modified such that one of the nuclease domains is mutated or fully or partially deleted to reduce its nucleic acid cleavage activity. In some embodiments, the Cas9 nuclease is modified to contain no functional RuvC-like nuclease domain. In other embodiments, the Cas9 nuclease is modified to contain no functional HNH-like nuclease domain. In some embodiments in which only one of the nuclease domains is functional, the Cas9 nuclease is a nickase that is capable of introducing a single-stranded break (a “nick”) into the target sequence. In some embodiments, a conserved amino acid within a Cas9 nuclease domain is substituted to reduce or alter a nuclease activity. In some embodiments, the Cas nuclease nickase comprises an amino acid substitution in the RuvC-like nuclease domain. Exemplary amino acid substitutions in the RuvC-like nuclease domain include D10A (based on the S. pyogenes Cas9 nuclease). In some embodiments, the nickase comprises an amino acid substitution in the HNH-like nuclease domain. Exemplary amino acid substitutions in the HNH-like nuclease domain include E762A, H840A, N863A, H983A, and D986A (based on the S. pyogenes Cas9 nuclease). One example is provided in Table 1 below (SEQ ID NO: 29).

In some embodiments, the Cas nuclease is from a Type-I CRISPR/Cas system. In some embodiments, the Cas nuclease is a component of the Cascade complex of a Type-I CRISPR/Cas system. For example, the Cas nuclease is a Cas3 nuclease. In some embodiments, the Cas nuclease is derived from a Type-III CRISPR/Cas system. In some embodiments, the Cas nuclease is derived from Type-IV CRISPR/Cas system. In some embodiments, the Cas nuclease is derived from a Type-V CRISPR/Cas system. In some embodiments, the Cas nuclease is derived from a Type-VI CRISPR/Cas system.

Guide RNAs (gRNAs)

The CRISPR technology involves the use of a genome-targeting nucleic acid that can direct the endonuclease to a specific target sequence within a target gene for gene editing at the specific target sequence. The genome-targeting nucleic acid can be a RNA. A genome-targeting RNA is referred to as a “guide RNA” or “gRNA” herein. A guide RNA comprises at least a spacer sequence that hybridizes to a target nucleic acid sequence within a target gene for editing, and a CRISPR repeat sequence.

In Type II systems, the gRNA also comprises a second RNA called the tracrRNA sequence. In the Type II gRNA, the CRISPR repeat sequence and tracrRNA sequence hybridize to each other to form a duplex. In the Type V gRNA, the crRNA forms a duplex. In both systems, the duplex binds a site-directed polypeptide, such that the guide RNA and site-direct polypeptide form a complex. In some embodiments, the genome-targeting nucleic acid provides target specificity to the complex by virtue of its association with the site-directed polypeptide. The genome-targeting nucleic acid thus directs the activity of the site-directed polypeptide.

As is understood by the person of ordinary skill in the art, each guide RNA is designed to include a spacer sequence complementary to its genomic target sequence. See Jinek et al., Science, 337, 816-821 (2012) and Deltcheva et al., Nature, 471, 602-607 (2011).

In some embodiments, the genome-targeting nucleic acid (e.g., gRNA) is a double-molecule guide RNA. In some embodiments, the genome-targeting nucleic acid (e.g., gRNA) is a single-molecule guide RNA.

A double-molecule guide RNA comprises two strands of RNA molecules. The first strand comprises in the 5′ to 3′ direction, an optional spacer extension sequence, a spacer sequence and a minimum CRISPR repeat sequence. The second strand comprises a minimum tracrRNA sequence (complementary to the minimum CRISPR repeat sequence), a 3′ tracrRNA sequence and an optional tracrRNA extension sequence.

A single-molecule guide RNA (referred to as a “sgRNA”) in a Type II system comprises, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension may comprise elements that contribute additional functionality (e.g., stability) to the guide RNA. The single-molecule guide linker links the minimum CRISPR repeat and the minimum tracrRNA sequence to form a hairpin structure. The optional tracrRNA extension comprises one or more hairpins. A single-molecule guide RNA in a Type V system comprises, in the 5′ to 3′ direction, a minimum CRISPR repeat sequence and a spacer sequence.

A spacer sequence in a gRNA is a sequence (e.g., a 20-nucleotide sequence) that defines the target sequence (e.g., a DNA target sequences, such as a genomic target sequence) of a target gene of interest. In some embodiments, the spacer sequence ranges from 15 to 30 nucleotides. For example, the spacer sequence may contain 15, 16, 17, 18, 19, 29, 21, 22, 23, 24, 25, 26, 27, 28, 29, or 30 nucleotides. In some embodiments, a spacer sequence contains 20 nucleotides.

The “target sequence” is in a target gene that is adjacent to a PAM sequence and is the sequence to be modified by an RNA-guided nuclease (e.g., Cas9). The “target sequence” is on the so-called PAM-strand in a “target nucleic acid,” which is a double-stranded molecule containing the PAM-strand and a complementary non-PAM strand. One of skill in the art recognizes that the gRNA spacer sequence hybridizes to the complementary sequence located in the non-PAM strand of the target nucleic acid of interest. Thus, the gRNA spacer sequence is the RNA equivalent of the target sequence. For example, if the target sequence is 5′-AGAGCAACAGTGCTGTGGCC**-3′ (SEQ ID NO: 18), then the gRNA spacer sequence is 5′-AGAGCAACAGUGCUGUGGCC**-3′ (SEQ ID NO: 3). The spacer of a gRNA interacts with a target nucleic acid of interest in a sequence-specific manner via hybridization (i.e., base pairing). The nucleotide sequence of the spacer thus varies depending on the target sequence of the target nucleic acid of interest.

In a CRISPR/Cas system herein, the spacer sequence is designed to hybridize to a region of the target nucleic acid that is located 5′ of a PAM recognizable by a Cas9 enzyme used in the system. The spacer may perfectly match the target sequence or may have mismatches. Each Cas9 enzyme has a particular PAM sequence that it recognizes in a target DNA. For example, S. pyogenes recognizes in a target nucleic acid a PAM that comprises the sequence 5′-NRG-3′, where R comprises either A or G, where N is any nucleotide and N is immediately 3′ of the target nucleic acid sequence targeted by the spacer sequence.

In some embodiments, the target nucleic acid sequence has 20 nucleotides in length. In some embodiments, the target nucleic acid has less than 20 nucleotides in length. In some embodiments, the target nucleic acid has more than 20 nucleotides in length. In some embodiments, the target nucleic acid has at least: 5, 10, 15, 16, 17, 18, 19, 20, 21, 22, 23, 24, 25, 30 or more nucleotides in length. In some embodiments, the target nucleic acid has at most: 5, 10, 15, 16, 17, 18, 19, 20, 21, 22, 23, 24, 25, 30 or more nucleotides in length. In some embodiments, the target nucleic acid sequence has 20 bases immediately 5′ of the first nucleotide of the PAM. For example, in a sequence comprising 5′-NNNNNNNNNNNNNNNNNNNNNRG-3′, the target nucleic acid can be the sequence that corresponds to the Ns, wherein N can be any nucleotide, and the underlined NRG sequence is the S. pyogenes PAM.

The guide RNA disclosed herein may target any sequence of interest via the spacer sequence in the crRNA. In some embodiments, the degree of complementarity between the spacer sequence of the guide RNA and the target sequence in the target gene can be about 60%, 65%, 70%, 75%, 80%, 85%, 90%, 95%, 97%, 98%, 99%, or 100%. In some embodiments, the spacer sequence of the guide RNA and the target sequence in the target gene is 100% complementary. In other embodiments, the spacer sequence of the guide RNA and the target sequence in the target gene may contain up to 10 mismatches, e.g., up to 9, up to 8, up to 7, up to 6, up to 5, up to 4, up to 3, up to 2, or up to 1 mismatch.

For any of the gRNA sequences provided herein, those that do not explicitly indicate modifications are meant to encompass both unmodified sequences and sequences having any suitable modifications.

The length of the spacer sequence in any of the gRNAs disclosed herein may depend on the CRISPR/Cas9 system and components used for editing any of the target genes also disclosed herein. For example, different Cas9 proteins from different bacterial species have varying optimal spacer sequence lengths. Accordingly, the spacer sequence may have 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 nucleotides in length. In some embodiments, the spacer sequence may have 18-24 nucleotides in length. In some embodiments, the targeting sequence may have 19-21 nucleotides in length. In some embodiments, the spacer sequence may comprise 20 nucleotides in length.

In some embodiments, the gRNA can be an sgRNA, which may comprise a 20 nucleotides spacer sequence at the 5′ end of the sgRNA sequence. In some embodiments, the sgRNA may comprise a less than 20 nucleotide spacer sequence at the 5′ end of the sgRNA sequence. In some embodiments, the sgRNA may comprise a more than 20 nucleotide spacer sequence at the 5′ end of the sgRNA sequence. In some embodiments, the sgRNA comprises a variable length spacer sequence with 17-30 nucleotides at the 5′ end of the sgRNA sequence. Examples are provided in Table 1 below. In these exemplary sequences, the fragment of “n” refers to the spacer sequence at the 5′ end.

In some embodiments, the sgRNA comprises comprise no uracil at the 3′ end of the sgRNA sequence. In other embodiments, the sgRNA may comprise one or more uracil at the 3′ end of the sgRNA sequence. For example, the sgRNA can comprise 1-8 uracil residues, at the 3′ end of the sgRNA sequence, e.g., 1, 2, 3, 4, 5, 6, 7, or 8 uracil residues at the 3′ end of the sgRNA sequence.

Any of the gRNAs disclosed herein, including any of the sgRNAs, may be unmodified. Alternatively, it may contain one or more modified nucleotides and/or modified backbones. For example, a modified gRNA such as an sgRNA can comprise one or more 2′-O-methyl phosphorothioate nucleotides, which may be located at either the 5′ end, the 3′ end, or both.

In certain embodiments, more than one guide RNAs can be used with a CRISPR/Cas nuclease system. Each guide RNA may contain a different targeting sequence, such that the CRISPR/Cas system cleaves more than one target nucleic acid. In some embodiments, one or more guide RNAs may have the same or differing properties such as activity or stability within the Cas9 RNP complex. Where more than one guide RNA is used, each guide RNA can be encoded on the same or on different vectors. The promoters used to drive expression of the more than one guide RNA is the same or different.

In some embodiments, the gRNAs disclosed herein target a Reg1 gene, for example, target a site within exon 1, exon 2, exon 3, exon 4, exon 5, or exon 6 of the Reg1 gene. Such a gRNA may comprise a spacer sequence complementary (complete or partially) to the target sequences in exon 2 or exon 4 of a Reg1 gene, or a fragment thereof. Exemplary target sequences of Reg1 and exemplary gRNA sequences are provided in Table 1 below.

In some embodiments, the gRNAs disclosed herein target a TGFBRII gene, for example, target a site within exon 1, exon 2, exon 3, exon 4, exon 5, or exon 6 of the TGFBRII gene. Such a gRNA may comprise a spacer sequence complementary (complete or partially) to the target sequences in exon 4 or exon 5 of a TGFBRII gene, or a fragment thereof. Exemplary target sequences of TGFBRII and exemplary gRNA sequences are provided in Table 1 below.

In some embodiments, the gRNAs disclosed herein target a β2M gene, for example, target a suitable site within a β2M gene. See also WO2019097305, the relevant disclosures of which are incorporated by reference herein for the purpose and subject matter referenced herein. Other gRNA sequences may be designed using the β2M gene sequence located on Chromosome 15 (GRCh38 coordinates: Chromosome 15: 44,711,477-44,718,877; Ensembl: ENSG00000166710). In some embodiments, gRNAs targeting the β2M genomic region and RNA-guided nuclease create breaks in the β2M genomic region resulting in Indels in the β2M gene disrupting expression of the mRNA or protein.

In some embodiments, the gRNAs disclosed herein target a TRAC gene. See also WO2019097305, the relevant disclosures of which are incorporated by reference herein for the subject matter and purpose referenced herein. Other gRNA sequences may be designed using the TRAC gene sequence located on chromosome 14 (GRCh38: chromosome 14: 22,547,506-22,552,154. Ensembl; ENSG00000277734). In some embodiments, gRNAs targeting the TRAC genomic region and RNA-guided nuclease create breaks in the TRAC genomic region resulting Indels in the TRAC gene disrupting expression of the mRNA or protein.

Exemplary spacer sequences and gRNAs targeting a β2M gene or TRAC gene are provided in Table 1 below.

By way of illustration, guide RNAs used in the CRISPR/Cas/Cpf1 system, or other smaller RNAs can be readily synthesized by chemical means, as illustrated below and described in the art. While chemical synthetic procedures are continually expanding, purifications of such RNAs by procedures such as high performance liquid chromatography (HPLC, which avoids the use of gels such as PAGE) tends to become more challenging as polynucleotide lengths increase significantly beyond a hundred or so nucleotides. One approach used for generating RNAs of greater length is to produce two or more molecules that are ligated together. Much longer RNAs, such as those encoding a Cas9 or Cpf1 endonuclease, are more readily generated enzymatically. Various types of RNA modifications can be introduced during or after chemical synthesis and/or enzymatic generation of RNAs, e.g., modifications that enhance stability, reduce the likelihood or degree of innate immune response, and/or enhance other attributes, as described in the art.

In some examples, the gRNAs of the present disclosure can be produced in vitro transcription (IVT), synthetic and/or chemical synthesis methods, or a combination thereof. Enzymatic (IVT), solid-phase, liquid-phase, combined synthetic methods, small region synthesis, and ligation methods are utilized. In one embodiment, the gRNAs are made using IVT enzymatic synthesis methods. Methods of making polynucleotides by IVT are known in the art and are described in WO2013/151666. Accordingly, the present disclosure also includes polynucleotides, e.g., DNA, constructs and vectors are used to in vitro transcribe a gRNA described herein.

Various types of RNA modifications can be introduced during or after chemical synthesis and/or enzymatic generation of RNAs, e.g., modifications that enhance stability, reduce the likelihood or degree of innate immune response, and/or enhance other attributes, as described in the art. In some embodiments, non-natural modified nucleobases can be introduced into any of the gRNAs disclosed herein during synthesis or post-synthesis. In certain embodiments, modifications are on internucleoside linkages, purine or pyrimidine bases, or sugar. In some embodiments, a modification is introduced at the terminal of a gRNA with chemical synthesis or with a polymerase enzyme. Examples of modified nucleic acids and their synthesis are disclosed in WO2013/052523. Synthesis of modified polynucleotides is also described in Verma and Eckstein, Annual Review of Biochemistry, vol. 76, 99-134 (1998).

In some embodiments, enzymatic or chemical ligation methods can be used to conjugate polynucleotides or their regions with different functional moieties, such as targeting or delivery agents, fluorescent labels, liquids, nanoparticles, etc. Conjugates of polynucleotides and modified polynucleotides are reviewed in Goodchild, Bioconjugate Chemistry, vol. 1(3), 165-187 (1990).

In some embodiments of the present disclosure, a CRISPR/Cas nuclease system for use in genetically editing any of the target genes disclosed here may include at least one guide RNA. In some examples, the CRISPR/Cas nuclease system may contain multiple gRNAs, for example, 2, 3, or 4 gRNAs. Such multiple gRNAs may target different sites in a same target gene. Alternatively, the multiple gRNAs may target different genes. In some embodiments, the guide RNA(s) and the Cas protein may form a ribonucleoprotein (RNP), e.g., a CRISPR/Cas complex. The guide RNA(s) may guide the Cas protein to a target sequence(s) on one or more target genes as those disclosed herein, where the Cas protein cleaves the target gene at the target site. In some embodiments, the CRISPR/Cas complex is a Cpf1/guide RNA complex. In some embodiments, the CRISPR complex is a Type-II CRISPR/Cas9 complex. In some embodiments, the Cas protein is a Cas9 protein. In some embodiments, the CRISPR/Cas9 complex is a Cas9/guide RNA complex.

In some embodiments, the indel frequency (editing frequency) of a particular CRISPR/Cas nuclease system, comprising one or more specific gRNAs, may be determined using a TIDE analysis, which can be used to identify highly efficient gRNA molecules for editing a target gene. In some embodiments, a highly efficient gRNA yields a gene editing frequency of higher than 80%. For example, a gRNA is considered to be highly efficient if it yields a gene editing frequency of at least 80%, at least 85%, at least 90%, at least 95%, or 100%.

Delivery of Guide RNAs and Nucleases to T Cells

The CRISPR/Cas nuclease system disclosed herein, comprising one or more gRNAs and at least one RNA-guided nuclease, optionally a donor template as disclosed below, can be delivered to a target cell (e.g., a T cell) for genetic editing of a target gene, via a conventional method. In some embodiments, components of a CRISPR/Cas nuclease system as disclosed herein may be delivered to a target cell separately, either simultaneously or sequentially. In other embodiments, the components of the CRISPR/Cas nuclease system may be delivered into a target together, for example, as a complex. In some instances, gRNA and a RNA-guided nuclease can be pre-complexed together to form a ribonucleoprotein (RNP), which can be delivered into a target cell.

RNPs are useful for gene editing, at least because they minimize the risk of promiscuous interactions in a nucleic acid-rich cellular environment and protect the RNA from degradation. Methods for forming RNPs are known in the art. In some embodiments, an RNP containing an RNA-guided nuclease (e.g., a Cas nuclease, such as a Cas9 nuclease) and one or more gRNAs targeting one or more genes of interest can be delivered a cell (e.g., a T cell). In some embodiments, an RNP can be delivered to a T cell by electroporation.

In some embodiments, an RNA-guided nuclease can be delivered to a cell in a DNA vector that expresses the RNA-guided nuclease in the cell. In other examples, an RNA-guided nuclease can be delivered to a cell in an RNA that encodes the RNA-guided nuclease and expresses the nuclease in the cell. Alternatively or in addition, a gRNA targeting a gene can be delivered to a cell as a RNA, or a DNA vector that expresses the gRNA in the cell.

Delivery of an RNA-guided nuclease, gRNA, and/or an RNP may be through direct injection or cell transfection using known methods, for example, electroporation or chemical transfection. Other cell transfection methods may be used. In some instances, a Cas9 enzyme may form one RNP with all of the gRNAs targeting the TRAC gene, the β2M gene, the Reg1 gene, and the TGFBRII gene and be delivered to T cells via one electroporation event. Alternatively, a Cas9 enzyme may form two or more RNPs, which collectively include all of the gRNAs targeting the TRAC gene, the β2M gene, the Reg1 gene, and the TGFBRII gene. The multiple RNPs may be delivered to the T cells via sequential electroporation events, for example, two sequential electroporation events.

Other Gene Editing Methods

Besides the CRISPR method disclosed herein, additional gene editing methods as known in the art can also be used in making the genetically engineered T cells disclosed herein. Some examples include gene editing approaching involve zinc finger nuclease (ZFN), transcription activator-like effector nucleases (TALEN), restriction endonucleases, meganucleases homing endonucleases, and the like.

ZFNs are targeted nucleases comprising a nuclease fused to a zinc finger DNA binding domain (ZFBD), which is a polypeptide domain that binds DNA in a sequence-specific manner through one or more zinc fingers. A zinc finger is a domain of about 30 amino acids within the zinc finger binding domain whose structure is stabilized through coordination of a zinc ion. Examples of zinc fingers include, but not limited to, C2H2 zinc fingers, C3H zinc fingers, and C4 zinc fingers. A designed zinc finger domain is a domain not occurring in nature whose design/composition results principally from rational criteria, e.g., application of substitution rules and computerized algorithms for processing information in a database storing information of existing ZFP designs and binding data. See, for example, U.S. Pat. Nos. 6,140,081; 6,453,242; and 6,534,261; see also WO 98/53058; WO 98/53059; WO 98/53060; WO 02/016536 and WO 03/016496. A selected zinc finger domain is a domain not found in nature whose production results primarily from an empirical process such as phage display, interaction trap or hybrid selection. ZFNs are described in greater detail in U.S. Pat. Nos. 7,888,121 and 7,972,854. The most recognized example of a ZFN is a fusion of the FokI nuclease with a zinc finger DNA binding domain.

A TALEN is a targeted nuclease comprising a nuclease fused to a TAL effector DNA binding domain. A “transcription activator-like effector DNA binding domain”, “TAL effector DNA binding domain”, or “TALE DNA binding domain” is a polypeptide domain of TAL effector proteins that is responsible for binding of the TAL effector protein to DNA. TAL effector proteins are secreted by plant pathogens of the genus Xanthomonas during infection. These proteins enter the nucleus of the plant cell, bind effector-specific DNA sequences via their DNA binding domain, and activate gene transcription at these sequences via their transactivation domains. TAL effector DNA binding domain specificity depends on an effector-variable number of imperfect 34 amino acid repeats, which comprise polymorphisms at select repeat positions called repeat variable-diresidues (RVD). TALENs are described in greater detail in US Patent Application No. 2011/0145940. The most recognized example of a TALEN in the art is a fusion polypeptide of the FokI nuclease to a TAL effector DNA binding domain.

Additional examples of targeted nucleases suitable for use as provided herein include, but are not limited to, Bxb1, phiC31, R4, PhiBT1, and WP/SPBc/TP901-1, whether used individually or in combination.

Any of the nucleases disclosed herein may be delivered using a vector system, including, but not limited to, plasmid vectors, DNA minicircles, retroviral vectors, lentiviral vectors, adenovirus vectors, poxvirus vectors; herpesvirus vectors and adeno-associated virus vectors, and combinations thereof.

Conventional viral and non-viral based gene transfer methods can be used to introduce nucleic acids encoding nucleases and donor templates in cells (e.g., T cells). Non-viral vector delivery systems include DNA plasmids, DNA minicircles, naked nucleic acid, and nucleic acid complexed with a delivery vehicle such as a liposome or poloxamer. Viral vector delivery systems include DNA and RNA viruses, which have either episomal or integrated genomes after delivery to the cell.

Methods of non-viral delivery of nucleic acids include electroporation, lipofection, microinjection, biolistics, virosomes, liposomes, immunoliposomes, polycation or lipid:nucleic acid conjugates, naked DNA, naked RNA, capped RNA, artificial virions, and agent-enhanced uptake of DNA. Sonoporation using, e.g., the Sonitron 2000 system (Rich-Mar) can also be used for delivery of nucleic acids. Some specific examples are provided below.

D. Delivery of Anti-CD19 CAR Construct to T Cells

In some embodiments, a nucleic acid encoding an anti-CD19 CAR can be introduced into any of the genetically engineered T cells disclosed herein by methods known to those of skill in the art. For example, a coding sequence of the anti-CD19 CAR may be cloned into a vector, which may be introduced into the genetically engineered T cells for expression of the anti-CD19 CAR. A variety of different methods known in the art can be used to introduce any of the nucleic acids or expression vectors disclosed herein into an immune effector cell. Non-limiting examples of methods for introducing nucleic acid into a cell include: lipofection, transfection (e.g., calcium phosphate transfection, transfection using highly branched organic compounds, transfection using cationic polymers, dendrimer-based transfection, optical transfection, particle-based transfection (e.g., nanoparticle transfection), or transfection using liposomes (e.g., cationic liposomes)), microinjection, electroporation, cell squeezing, sonoporation, protoplast fusion, impalefection, hydrodynamic delivery, gene gun, magnetofection, viral transfection, and nucleofection.

In specific examples, a nucleic acid encoding an anti-CD19 CAR construct can be delivered to a cell using an adeno-associated virus (AAV). AAVs are small viruses which integrate site-specifically into the host genome and can therefore deliver a transgene, such as the anti-CD19 CAR. Inverted terminal repeats (ITRs) are present flanking the AAV genome and/or the transgene of interest and serve as origins of replication. Also present in the AAV genome are rep and cap proteins which, when transcribed, form capsids which encapsulate the AAV genome for delivery into target cells. Surface receptors on these capsids which confer AAV serotype, which determines which target organs the capsids will primarily bind and thus what cells the AAV will most efficiently infect. There are twelve currently known human AAV serotypes. In some embodiments, the AAV for use in delivering the anti-CD19 CAR-coding nucleic acid is AAV serotype 6 (AAV6).

Adeno-associated viruses are among the most frequently used viruses for gene therapy for several reasons. First, AAVs do not provoke an immune response upon administration to mammals, including humans. Second, AAVs are effectively delivered to target cells, particularly when consideration is given to selecting the appropriate AAV serotype. Finally, AAVs have the ability to infect both dividing and non-dividing cells because the genome can persist in the host cell without integration. This trait makes them an ideal candidate for gene therapy.

A nucleic acid encoding a CAR can be designed to insert into a genomic site of interest in the host T cells. In some embodiments, the target genomic site can be in a safe harbor locus.

In some embodiments, a nucleic acid encoding an anti-CD19 CAR (e.g., via a donor template, which can be carried by a viral vector such as an adeno-associated viral (AAV) vector) can be designed such that it can insert into a location within a TRAC gene to disrupt the TRAC gene in the genetically engineered T cells and express the CAR polypeptide. Disruption of TRAC leads to loss of function of the endogenous TCR. For example, a disruption in the TRAC gene can be created with an endonuclease such as those described herein and one or more gRNAs targeting one or more TRAC genomic regions. Any of the gRNAs specific to a TRAC gene and the target regions disclosed herein can be used for this purpose.

In some examples, a genomic deletion in the TRAC gene and replacement by an anti-CD19 CAR coding segment can be created by homology directed repair or HDR (e.g., using a donor template, which may be part of a viral vector such as an adeno-associated viral (AAV) vector). In some embodiments, a disruption in the TRAC gene can be created with an endonuclease as those disclosed herein and one or more gRNAs targeting one or more TRAC genomic regions and inserting an anti-CD19 CAR coding segment into the TRAC gene.

A donor template as disclosed herein can contain a coding sequence for an anti-CD19 CAR. In some examples, the anti-CD19 CAR-coding sequence may be flanked by two regions of homology to allow for efficient HDR at a genomic location of interest, for example, at a TRAC gene using a gene editing method known in the art. In some examples, a CRISPR-based method can be used. In this case, both strands of the DNA at the target locus can be cut by a CRISPR Cas9 enzyme guided by gRNAs specific to the target locus. HDR then occurs to repair the double-strand break (DSB) and insert the donor DNA coding for the CAR. For this to occur correctly, the donor sequence is designed with flanking residues which are complementary to the sequence surrounding the DSB site in the target gene (hereinafter “homology arms”), such as the TRAC gene. These homology arms serve as the template for DSB repair and allow HDR to be an essentially error-free mechanism. The rate of homology directed repair (HDR) is a function of the distance between the mutation and the cut site so choosing overlapping or nearby target sites is important. Templates can include extra sequences flanked by the homologous regions or can contain a sequence that differs from the genomic sequence, thus allowing sequence editing.

Alternatively, a donor template may have no regions of homology to the targeted location in the DNA and may be integrated by NHEJ-dependent end joining following cleavage at the target site.

A donor template can be DNA or RNA, single-stranded and/or double-stranded, and can be introduced into a cell in linear or circular form. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al., (1987) Proc. Natl. Acad. Sci. USA 84:4959-4963; Nehls et al., (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A donor template can be introduced into a cell as part of a vector molecule having additional sequences such as, for example, replication origins, promoters and genes encoding antibiotic resistance. Moreover, a donor template can be introduced into a cell as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

A donor template, in some embodiments, can be inserted at a site nearby an endogenous prompter (e.g., downstream or upstream) so that its expression can be driven by the endogenous promoter. In other embodiments, the donor template may comprise an exogenous promoter and/or enhancer, for example, a constitutive promoter, an inducible promoter, or tissue-specific promoter to control the expression of the CAR gene. In some embodiments, the exogenous promoter is an EF1α promoter, see, e.g., SEQ ID NO: 90 provided in Table 6 below. Other promoters may be used.

Furthermore, exogenous sequences may also include transcriptional or translational regulatory sequences, for example, promoters, enhancers, insulators, internal ribosome entry sites, sequences encoding 2A peptides and/or polyadenylation signals.

In some embodiments, a donor template for delivering an anti-CD19 CAR may be an AAV vector inserted with a nucleic acid fragment comprising the coding sequence of the anti-CD19 CAR, and optionally regulatory sequences for expression of the anti-CD19 CAR (e.g., a promoter such as the EF1α promoter provided in the sequence Table 6), which can be flanked by homologous arms for inserting the coding sequence and the regulatory sequences into a genomic locus of interest. In some examples, the nucleic acid fragment is inserted in the endogenous TRAC gene locus, thereby disrupting expression of the TRAC gene. In specific examples, the nucleic acid may replace a fragment in the TRAC gene, for example, a fragment comprising the nucleotide sequence of SEQ ID NO: 18. In some specific examples, the donor template for delivering the anti-CD19 CAR may comprise a nucleotide sequence of SEQ ID NO: 90, which can be inserted into a disrupted TRAC gene, for example, replacing the fragment of SEQ ID NO: 18.

E. Exemplary Anti-CD19 CAR-T Cells with Multiple Genetic Edits

It should be understood that gene disruption encompasses gene modification through gene editing (e.g., using CRISPR/Cas gene editing to insert or delete one or more nucleotides). A disrupted gene may contain one or more mutations (e.g., insertion, deletion, or nucleotide substitution, etc.) relative to the wild-type counterpart so as to substantially reduce or completely eliminate the activity of the encoded gene product. The one or more mutations may be located in a non-coding region, for example, a promoter region, a regulatory region that regulates transcription or translation; or an intron region. Alternatively, the one or more mutations may be located in a coding region (e.g., in an exon). In some instances, the disrupted gene does not express or expresses a substantially reduced level of the encoded protein. In other instances, the disrupted gene expresses the encoded protein in a mutated form, which is either not functional or has substantially reduced activity. In some embodiments, a disrupted gene is a gene that does not encode functional protein. In some embodiments, a cell that comprises a disrupted gene does not express (e.g., at the cell surface) a detectable level (e.g. by antibody, e.g., by flow cytometry) of the protein encoded by the gene. A cell that does not express a detectable level of the protein may be referred to as a knockout cell. For example, a cell having a β2M gene edit may be considered a β2M knockout cell if β2M protein cannot be detected at the cell surface using an antibody that specifically binds β2M protein. On the other hand, a cell is deemed positive (+) in expressing a surface receptor (e.g., an anti-CD19 CAR) when the surface expression of such a receptor can be detected via a routine method, e.g., by flow cytometry or immune staining.

In some embodiments, a population of genetically engineered T cells disclosed herein express an anti-CD19 CAR as those disclosed herein (e.g., comprising the amino acid sequence of SEQ ID NO:73 or SEQ ID NO: 74, a disrupted TRAC gene, a disrupted b2M gene, a disrupted Reg1 gene, and a disrupted TGFBRII gene. The nucleotide sequence encoding the anti-CD19 CAR may be inserted in the disrupted TRAC gene (e.g., replacing the site targeted by a sgRNA listed in Table 1 below).

The population of genetically engineered T cells disclosed herein may comprise a disrupted β2M gene, which comprises one or more sequences listed in Table 2, a disrupted TGFBRII gene, which comprises one or more sequences listed in Table 3, and/or a disrupted Reg1 gene, which comprises one or more sequences listed in Table 4.

In some examples, such a population of genetically engineered T cells disclosed herein such as CTX112 cells disclosed in Example 12 below may comprise a high level of TCRαβ⁻ T cells (e.g., ranging from about 70% to about 99%; mean about 91%) and a high level of β2M⁻ T cells (e.g., ranging from 50% to about 85%; mean about 71%). Alternatively or in addition, the population of genetically engineered T cells disclosed herein such as CTX112 cells may exhibit a high level of TGFBRII disruption (e.g., ranging from 45% to about 88% indels; mean about 71% indels) and a high level of Reg1 disruption (e.g., ranging from 54% to about 97% indels, mean about 87% indels). Alternatively or in addition, the population of genetically engineered T cells disclosed herein may exhibit a high level of CAR incorporation (e.g., ranging from 16% to about 85%, mean about 46%).

In some examples, a population of genetically engineered T cells as disclosed herein such as the CTX112 cells may comprise about 50%-99% (e.g., about 55% to about 80%) CAR⁺ T cells, about 90%-99.9% (e.g., about 95% to about 99.7%) TCR⁻ T cells, about 60% to about 90% (e.g., about 70% to about 80%) β2M⁻ T cells, about 70% to about 90% (e.g., about 80% to about 90%) of TGFBRII⁻ T cells, and/or about 90% to about 98% (e.g., about 95% to about 98%) Reg1⁻ T cells. In some instances, the population of genetically engineered T cells may comprise about 80% to about 90% (e.g., about 85%) of indel frequency in the TGFBRII gene. Alternatively or in addition, the population of genetically engineered T cells may comprise about 90% to about 97% (e.g., about 95%) indel frequency in the Reg1 gene.

Any of the anti-CD19 CAR-T cells disclosed herein may be suspended in a cryopreservation solution (e.g., CryoStor® C55 or CyroStor® CS10) to form a pharmaceutical composition. The cryopreservation solution for use in the present disclosure may also comprise adenosine, dextrose, dextran-40, lactobionic acid, sucrose, mannitol, a buffer agent such as N-)2-hydroxyethyl) piperazine-N′-(2-ethanesulfonic acid) (HEPES), one or more salts (e.g., calcium chloride, magnesium chloride, potassium chloride, potassium bicarbonate, potassium phosphate, etc.), one or more base (e.g., sodium hydroxide, potassium hydroxide, etc.), or a combination thereof. Components of a cryopreservation solution may be dissolved in sterile water (injection quality). Any of the cryopreservation solution may be substantially free of serum (undetectable by routine methods).

In some examples, the genetically engineered anti-CD19 CAR⁺ T cells disclosed herein such as CTX112 cells may be suspended in normal saline containing human serum albumin and CryoSore® CS10 (containing about 10% DMSO) sequentially, which form the cryopreservation solution.

### II. Allogeneic CAR-T Cell Therapy of B-Cell Malignancies

CD19 is a type I transmembrane protein and belongs to the immunoglobulin (Ig) superfamily. CD19 is typically found in a complex with CD81 and CD21 and plays important roles in B cell activation and signaling. CD19 is expressed in B cells and follicular dendritic cells. Genetic perturbations of CD19 in mice or humans lead to defects in B cell function and impaired immune responses (Wang et al., 2012, Exp Hematol Oncol 1, 36). CD19 is expressed by most B cell leukemia and lymphoma cells and has been the target of several therapeutic modalities, including antibodies such as blinatumomab, tafasitamab, and CD19-directed CAR T cells.

In some aspects, provided herein are methods for treating a human patient having a B cell malignancy (e.g., relapsed and/or refractory) using a population of any of the genetically engineered anti-CD19 CAR T cells such as the CTX112 T cells as disclosed herein. The allogeneic anti-CD19 CAR T cell therapy may comprise two stages of treatment: (i) a conditioning regimen (lymphodepleting treatment), which comprises giving one or more doses of one or more lymphodepleting agents to a suitable human patient, and (ii) a treatment regimen (allogeneic anti-CD19 CAR T cell therapy), which comprises administration of the population of allogeneic anti-CD19 CAR T cells such as the CTX112 T cells as disclosed herein to the human patient. In some instances, one or more additional doses of the anti-CD19 CAR-T cells may be administered to the human patient with or without accompanying lymphodepletion treatment.

(i) Patient Population

A human patient may be any human subject for whom diagnosis, treatment, or therapy is desired. A human patient may be of any age. In some embodiments, the human patient is an adult (e.g., a person who is at least 18 years old). In some examples, the human patient may have a body weight of 50 kg or higher. In some embodiments, the human patient can be a child.

A human patient to be treated by the methods described herein can be a human patient having, suspected of having, or a risk for having a B cell malignancy. A subject suspected of having a B cell malignancy might show one or more symptoms of B cell malignancy, e.g., unexplained weight loss, fatigue, night sweats, shortness of breath, or swollen glands. A subject at risk for a B cell malignancy can be a subject having one or more of the risk factors for B cell malignancy, e.g., a weakened immune system, age, male, or infection (e.g., Epstein-Barr virus infection). A human patient who needs the anti-CD19 CAR T cell (e.g., CTX112 T cell) treatment may be identified by routine medical examination, e.g., physical examination, laboratory tests, biopsy (e.g., bone marrow biopsy and/or lymph node biopsy), magnetic resonance imaging (MRI) scans, or ultrasound exams.

The human patient subject to the treatment disclosed herein may have a non-Hodgkin lymphoma (NHL) such as Diffuse large B cell lymphoma (DLBCL), follicular lymphoma (FL), Marginal Zone Lymphoma (MZL), Mantle cell lymphoma (MCL), Chronic lymphocytic leukemia (CLL) and small lymphocytic lymphoma (SLL), Follicular Lymphoma Grade 3b and Transformed Lymphomas, or other types relapsed/refractory B-cell malignancies as disclosed herein or known in the art.

Non-Hodgkin Lymphoma (NHLs)

In some embodiments, the CD19+ B cell malignancy is a non-Hodgkin lymphoma (NHL), which are a heterogeneous group of malignancies originating from B lymphocytes, T lymphocytes, or natural killer (NK) cells. The World Health Organization defines more than 60 different subcategories of NHL based on cell type in which the cancer originates, histology, mutational profiling, and protein markers on the cellular surface, and NHL is the 10th most common malignancy worldwide (Chihara et al., 2015, Expert Rev Anticancer Ther 15, 531-544; Trask et al., 2012, Blood 120, 5074-5074). NHL accounts for 4.3% of all new cancer cases reported and is the 8th leading cause of cancer deaths in the United States. The major subtypes of NHL include diffuse large B cell lymphoma (DLBCL), chronic lymphocytic leukemia (CLL), and follicular lymphoma (FL; (Teras et al., 2016, CA Cancer J Clin 66, 443-459; and Trask et al., 2012). CD19 expression is ubiquitous on B cell malignancies and maintained among indolent and aggressive subtypes of NHL (Scheuermann and Racila, 1995, Leuk Lymphoma 18, 385-397), which has contributed to the increase of development of CD19-directed therapies in these indications.

Follolular Lymphma (FL)

In some embodiments, the B cell malignancy is a follicular lymphoma (FL) such as a low-grade (e.g., 1-3a). Follicular lymphoma (FL) accounts for 35% of NHLs diagnosed in the US and Western Europe, and is the most common indolent lymphoma (Freedman and Jacobsen, 2020). The disease course is characterized by initial response to therapies followed by relapse and, at times, transformation to a more aggressive form of lymphoma. The 10-year overall survival (OS) rate for FL has improved since anti-CD20 therapy (rituximab) became available more than 2 decades ago, with OS of approximately 80% at 10 years after diagnosis of grade 1-3a FL. However, lymphoma was still the leading cause of death in the first 10 years for these patients, with a 13.3% cumulative risk of mortality from lymphoma and treatment-related causes (Sarkozy et al., 2019, J Clin Oncol 37, 144-152). Approximately 20% of FL patients experience relapse within 2 years of first-line therapy, and these patients have an increased risk of death within 5 years after diagnosis (Casulo et al., 2015, J Clin Oncol 33, 2516-2522). Patients who experience disease progression within 24 months of front-line chemotherapy (POD24) have poor OS compared to patients without progression at 24 months (Casulo et al., 2022, Blood 139, 1684-1693).

FL is graded based on histologic assessment and proportion of centrocytes to centroblasts (Swerdlow et al., 2016), with grades 1, 2, and 3a generally considered low-grade disease, whereas grade 3b is a more aggressive form and is treated similarly to DLBCL (Dada, 2019, Eur J Haematol 103, 152-163) (Section 1.2.6).

For low-grade FL, a minority of patients (less than 10%) are diagnosed early when disease spread is limited (stage I/II); for these patients the recommended first line of therapy is radiation, with 10-year OS rates of up to 80% and median survival of approximately 19 years (Freedman and Jacobsen, 2020). For the majority of patients who have advanced stage disease at diagnosis (stage III/IV), the common approach is watchful waiting until treatment is warranted due to high tumor burden or signs of progression such as B symptoms (fever, night sweats, or weight loss), symptomatic nodal disease, or cytopenias. First-line therapy is typically an anti-CD20-directed therapy (rituximab or obinutuzumab) in combination with chemotherapy (cyclophosphamide, doxorubicin hydrochloride, vincristine sulfate, and prednisone [CHOP] or bendamustine) (Dada, 2019; Freedman and Jacobsen, 2020). In a study comparing rituximab+bendamustine to rituximab+CHOP for indolent and mantle cell lymphomas (MCL), the 10-year survival rates were 71% and 66%, respectively (Rummel et al., 2017, Journal of Clinical Oncology 35, 7501-7501). The objective response rate (ORR) was similar at 93% and 91%, respectively, and complete response (CR) rate was 40% and 30%, respectively (Rummel et al., 2013). Alternatively, patients may receive rituximab alone as first-line therapy, or a combination of lenalidomide and rituximab. Rituximab or obinutuzumab may also be used alone as maintenance or consolidation therapy (Dada, 2019; and Freedman and Jacobsen, 2020, Am J Hematol 95, 316-327). Second-line therapy for low-grade FL is similar to first line. If relapse occurs within 24 months, a different anti-CD20 or chemotherapy regimen is used, whereas if relapse occurs >24 months, the second-line regimen is the same as the first line (Dada, 2019). Autologous or allogeneic hematopoietic cell transplantation may be an option for some patients, though the risks of second malignancies and of myeloablative conditioning make these options controversial (Freedman and Jacobsen, 2020).

Later lines of therapy for relapsed/refractory patients include kinase inhibitors, such as idelalisib, copanlisib, and duvelisib. The overall response rate to idelalisib was 57% and median duration of response (DOR) 12.5 months, and other kinase inhibitors show similar efficacy. Toxicities include infection, myelosuppression, and inflammatory conditions (Freedman and Jacobsen, 2020). Also, autologous CAR T cell therapies have been explored for relapsed/refractory patients. Axicabtagene ciloleucel (Yescarta®, Kite Pharma), an anti-CD19 autologous CAR T cell therapy, received accelerated approval for adult patients with relapsed/refractory FL in 2021. It showed a 91% ORR and 60% CR in 81 subjects with grade 1 3a FL. Grade ≥3 CRS was observed in 8% and grade ≥3 neurotoxicity was observed in 15% of subjects in the ZUMA-5 study, which included subjects with FL (n=124). In addition, tisagenlecleucel (KYMRIAH®) another anti-CD19 autologous CAR T cell therapy, received approval for adult patients with relapsed/refractory FL in May 2022. It showed a 86% ORR and 69% CR in 94 subjects with FL. No grade ≥3 CRS was observed and grade ≥3 ICANS was observed in 1% of subjects in the ELARA study.

Marginal Zone Lymphoma

Marginal zone lymphoma (MZL) represents 5% to 10% of NHLs in adults and is classified into 3 subtypes based on where the malignancy originates: extranodal MZL in the mucosa-associated lymphoid tissue (MALT), splenic MZL in the spleen, and nodal MZL in the lymph nodes. MZL is thought to be induced by prolonged stimulation of B cell receptors by infection or autoimmune diseases. Like FL, MZL is an indolent lymphoma with a prolonged disease course (Juirez-Salcedo and Castillo, 2019).

Extranodal MZL, also known as MALT lymphoma, is the most common MZL subtype and occurs in the stomach or small intestine in more than half of cases, but may also affect the eye, bronchial mucosa, skin, salivary glands, or thyroid gland (Juárez-Salcedo and Castillo, 2019). Five-year event-free survival rates range from 29% to 70% depending on the patient's prognostic index risk level. High-risk patients have a median progression-free survival (PFS) of 2.6 years and median OS of 6.6 years (Thieblemont et al., 2017, Blood, 130, 1409-1417).

Splenic MZL is rare, accounting for less than 2% of all lymphomas (Juirez-Salcedo and Castillo, 2019). Median OS is more than 10 years for most patients; however, the disease follows a more aggressive course in about a third of patients, who die within 4 years. Nodal MZL is the least common MZL subtype and typically presents as a disseminated disease involving the peripheral, abdominal, and thoracic lymph nodes (Juirez-Salcedo and Castillo, 2019).

For relapsed/refractory patients with MZL (all subtypes), treatments include bendamustine with obinutuzumab (ORR of 79% and CR of 17% for subjects with indolent NHL refractory to rituximab in the GADOLIN study); lenalidomide with rituximab (ORR of 65% and CR of 29% for subjects with relapsed/refractory MZL in the AUGMENT study); the Bruton tyrosine kinase (BTK) inhibitor ibrutinib (ORR of 48% and CR of 3% with median PFS of 14.2 months in subjects with relapsed/refractory MZL in the pivotal study for US Food and Drug Administration [FDA] approval); and phosphoinositide 3-kinase (PI3K) inhibitors idelalisib (ORR of 57% and CR of 6% in subjects with relapsed/refractory indolent NHL), duvelisib (ORR of 39% in subjects with MZL), and copanlisib (ORR of 58.5% in subjects with relapsed/refractory indolent NHL and 13% CR among the subjects with MZL in the CHRONOS-1 study) (Sindel et al., 2019, Curr Treat Options Oncol 20, 90).

Novel therapies in development for patients with relapsed/refractory MZL include the PI3K inhibitors parsaclisib and umbralisib and the B cell lymphoma 2 (BCL-2) inhibitor venetoclax (Sindel et al., 2019). Also, anti-CD19 autologous CAR T cell therapies have been explored for relapsed/refractory MZL, though there are currently no approved CAR T cell therapies for this indication. Axicabtagene ciloleucel showed an ORR of 85% and CR of 60% in subjects with MZL (n=20; extranodal and nodal MZL only, per inclusion criteria) in the ZUMA-5 study. Grade ≥3 CRS was observed in 9% of subjects with MZL and grade ≥3 neurotoxicity observed in 41% (Jacobson et al., 2020, Blood 136, 40-41; Sandoval-Sus and Chavez, 2021, Ther Adv Hematol 12, 20406207211017788).

Mantle Cell Lymphoma Mantle cell lymphoma (MCL) accounts for 6% of all NHL in the US, and 7% to 9% in Europe (Sandoval-Sus et al., 2017). It may present as an indolent or aggressive disease, with most patients diagnosed at an advanced stage. Although initial response rates are generally high, early relapses are frequent (Silkenstedt et al., 2021, Br J Haematol). Median OS from the initiation of therapy is 3 to 5 years (Dreyling et al., 2018, Leuk Lymphoma 59, 1814-1828). Prognostic factors for shorter OS include older age, advanced performance status, elevated lactate dehydrogenase (LDH), high white blood cell count, and higher proliferative rates as defined by Ki-67 immunostaining (Dreyling et al., 2014, Clinical Cancer Research 20, 5194-5206; Sandoval-Sus et al., 2017, Hematol Oncol Stem Cell Ther 10, 99-115).

Autologous stem cell transplantation (ASCT) after an induction combining rituximab and cytarabine (Ara-C) chemotherapy has become a validated therapeutic approach in younger patients (Cheminant et al., 2020, Annals of Lymphoma 4). The Nordic MCL2 study, which included 6 alternating courses of chemoimmunotherapy consisting of rituximab, cyclophosphamide, doxorubicin, vincristine, and prednisone (R-CHOP) and R-Ara-C followed by ASCT, showed results of 96% ORR and 54% CR rates after induction, which led to a median OS of 12.7 years and PFS of 8.5 years (Eskelund et al., 2016, British Journal of Haematology 175, 410-418). R-CHOP induction followed by maintenance therapy with rituximab has been shown to be effective for older patients (60 years of age or older) with MCL; among patients who had a response to R-CHOP, maintenance therapy with rituximab significantly improved OS (4-year survival rate, 87%, vs. 63% with interferon alfa; P=0.005) (Kluin-Nelemans et al., 2012).

Recent therapies using targeted approaches such as BTK and BCL-2 inhibitors have provided durable therapeutic responses. However, the optimum combination and sequencing of these therapies is unclear (Hanel and Epperla, 2020). Despite advances in treatments, there is still a high unmet need for effective treatment for patients with relapsed/refractory MCL (Dreyling et al., 2018, Leuk Lymphoma 59, 1814-1828).

The CD19-directed autologous CAR T cell therapy brexucabtagene autoleucel (Tecartus®, Kite Pharma), received accelerated approval in the US in 2020 for relapsed/refractory MCL. It showed a 93% ORR and 67% CR rate in subjects with relapsed/refractory MCL (median 3 prior lines of therapy) in the Phase 2 ZUMA-2 study (n=68; 85% ORR and 59% CR rate in the intention-to-treat analysis of all 74 subjects), with an estimated 61% PFS and 83% OS at 12 months. Grade 3 or 4 CRS was reported in 15% of subjects (10/68) and grade 3 or 4 neurotoxicity was reported in 31% of subjects (21/68). These results suggest an opportunity for an allogeneic CAR T cell therapy to serve the unmet medical need in patients with relapsed/refractory MCL while also providing improved manufacturing processes and immediate availability.

### Chronic Lymphocytic Leukemia/Small Lymphocytic Lymphoma

Chronic lymphocytic leukemia (CLL) and small lymphocytic lymphoma (SLL) are manifestations of the same disease in which abnormal lymphocytes accumulate in the bone marrow and lymphoid tissues, as well as in peripheral blood for CLL and in lymph nodes for SLL (Wierda et al., 2020, J Natl Compr Canc Netw 18, 185-217). CLL is the most common adult leukemia in Western countries, representing an estimated 1.1% of all new cancer cases in the US in 2021 (NCI, 2022a, seer.cancer.gov/statfacts/html/clyl.html; and Wierda et al., 2020).

Most CLL/SLL patients are diagnosed when asymptomatic, and treatment is not initiated until disease progression or the appearance of symptoms (Hallek et al., 2018b). Preferred regimens for first-line therapy involve BTK inhibitors such as acalabrutinib (with or without the anti-CD20 monocolonal antibody obinutuzumab), ibrutinib, or zanubrutinib, or the BCL-2 inhibitor venetoclax in combination with obinutuzumab. Recommended therapies for relapsed/refractory patients include BTK inhibitor monotherapy or venetoclax in combination with rituximab (NCCN, 2022).

CD19-directed autologous CAR T cell therapy has shown promise in relapsed/refractory patients. In a long-term follow up of axicabtagene ciloleucel in subjects with B cell malignancies, CLL subjects showed an ORR of 88% (7/8 subjects) and CR of 63% (5/8 subjects); 50% of the CLL subjects had a DOR >3 years (Cappell et al., 2020, J Clin Oncol 38, 3805-3815). In a study of lisocabtagene maraleucel in subjects with CLL with a median of 5 prior lines of therapy, an objective response was achieved in 82% (18/22 subjects), with a best response of CR in 45% (10/22 subjects) (Siddiqi et al., 2022, Blood 139, 1794-1806). However, a challenge for autologous CAR T cell therapy in CLL is that patients may lack adequate quantities of competent T cells for harvest due to defects in CLL immune cells or the effect of previous therapies (Kharfan-Dabaja et al., 2021, Transplant Cell Ther). This challenge could be overcome with the use of allogeneic CAR T cells from healthy donors.

Diffuse Large B Cell Lymphoma

Diffuse large B cell lymphoma (DLBCL) is the most common type of NHL, accounting for 30% to 40% of diagnosed cases (Sehn and Gascoyne, 2015). Approximately 30% to 50% achieve cure with the standard of care first-line chemoimmunotherapy R-CHOP (Coiffier et al., 2010; Maurer et al., 2016). However, approximately 20% are refractory to R CHOP and 30% relapse following CR (Maurer et al., 2016, Am J Hematol 91, 1096-1101).

In patients who relapse after standard of care treatment with R-CHOP, the best long-term clinical outcomes are achieved through autologous HSCT. For patients who are healthy enough to undergo this procedure and have chemosensitive disease, commonly used induction chemotherapy consists of either R-ICE (rituximab, ifosfamide, carboplatin, and etoposide) or R DHAP (rituximab, dexamethasone, cytarabine, and cisplatin). In a large randomized study of R ICE vs R-DHAP in transplant-eligible subjects with DLBCL (the CORAL study), 63% of subjects achieved an objective response to either regimen, with a 26% CR rate (Gisselbrecht et al., 2010). For patients who are not eligible for autologous HSCT, treatment options have historically been limited and focused on palliation (Friedberg, 2011, Hematology Am Soc Hematol Educ Program 2011, 498-505; Maurer et al., 2016; Raut and Chakrabarti, 2014, South Asian J Cancer 3, 66-70).

For patients with progressive disease (PD) after 2 or more lines of therapy, prognosis continues to be poor, with an estimated median survival of 3 months for patients who relapse following autologous HSCT (Friedberg, 2011). In those cases, allogeneic HSCT is an option but only for the limited number of patients who qualify based on performance status and presence of chemosensitive disease (Raut and Chakrabarti, 2014; Sarkozy et al., 2015, Lancet Oncol 16, e555-e567).

The poor outcomes in the relapsed/refractory population were further highlighted in the SCHOLAR-1 study, which retrospectively analyzed survival and response rates in more than 600 subjects with relapsed/refractory DLBCL, transformed FL, and primary mediastinal large B cell lymphoma (PMBCL). The outcomes demonstrated an overall response rate of 26% (CR rate of 7%) and median OS of 6.3 months, further emphasizing the need for curative treatment options in this setting (Crump et al., 2017, Blood 130, 1800-1808).

### Follicular Lymphoma Grade 3b and Transformed Lymphomas

FL grade 3b is considered a biologically distinct entity, with frequent absence of t(14;18) and CD10 expression, and increased p53 and MUM1/IRF4 expression (Horn et al., 2011, Haematologica 96, 1327-1334). A large retrospective analysis of more than 500 FL cases further confirmed that the clinical course of FL grade 3a is similar to FL grade 1-2, whereas FL grade 3b has a clinical course more similar to that of DLBCL (Kahl and Yang, 2016, Blood 127, 2055-2063; Wahlin et al., 2012, Br J Haematol 156, 225-233). Because of this, FL grade 3b is typically managed similarly to DLBCL (Kahl and Yang, 2016).

Patients with FL are at risk of transformation to an aggressive form of lymphoma at a rate of 9% to 17% at 5 years and 15% to 30% at 10 years, as observed in retrospective studies (Bains et al., 2013, Ann Oncol 24, 428-432; Giné et al., 2006, Ann Oncol 17, 1539-1545). Initial treatment approaches are driven by prior therapies; however, even in the rituximab era, the outcomes are poor (Fischer et al., 2018, Ann Hematol 97, 17-29). A study comparing outcomes of subjects with DLBCL and FL found that subjects with FL who relapsed with transformed disease had worse outcomes compared with subjects diagnosed with de novo DLBCL (median survival 2 years vs 6 years, P<0.0003; (Jack et al., 2013, Blood 122, 78). Treatment options for relapsed/refractory transformed FL remain similar to DLBCL, with comparably poor outcomes, further underlining the unmet medical need in this patient population.

Patients with MZL may also experience transformation into aggressive lymphoma, though it is less common than in FL, with MZL transformation rates of 4% to 13% reported in the literature. Transformation is a risk factor for shortened survival, particularly if transformation occurs within 12 months of MZL diagnosis. Treatment strategies for transformed MZL have not been well studied and the therapy approach is often similar to treatment for transformed FL or DLBCL (Alderuccio and Lossos, 2020).

Likewise, patients with MCL who have the classic MCL variant at diagnosis may experience transformation to the more aggressive blastoid or pleomorphic MCL variants during the course of the disease. Patients with these variants rarely achieve durable remission with currently available therapies, and clinical studies for newer agents and cellular therapy, including next-generation CAR T cell therapies, may be their best option for treatment (Jain and Wang, 2020).

A human patient to be treated by methods described herein may be a human patient that has relapsed following a treatment and/or that has been become resistant to a treatment and/or that has been non-responsive to a treatment. As used herein, “relapsed” or “relapses” refers to a B cell malignancy such as those disclosed herein that returns following a period of complete response. Progressive disease refers to an instance when a disease worsens after the last evaluation (e.g., stable disease or partial response). In some embodiments, progression occurs during the treatment. In some embodiments, relapse occurs after the treatment. A lack of response may be determined by routine medical practice. For example, the human patient to be treated by methods described herein may be a human patient that has had one or more lines of prior anti-cancer therapies. In some instances, the human patient may have undergone two or more lines of prior anti-cancer therapies, e.g., a chemotherapy, an immunotherapy, a surgery, or a combination thereof. In some examples, the prior anti-cancer therapies may comprise an anti-CD20 antibody therapy, an anthracycline-containing therapy, or a combination thereof. See, e.g., Table 12 in Example 12 below, each of the patient populations listed therein is within the scope of the present disclosure.

In some instances, the human patient has a refractory B cell malignancy. As used herein, “refractory” refers to a B cell malignancy such as those disclosed herein that does not respond to or becomes resistant to a treatment. A human patient having a refractory B cell malignancy may have progressive disease on last therapy, or has stable disease following at least two cycles of therapy with duration of stable disease of up to 6 months (e.g., up to 5 months, up to 4 months, or up to 3 months or up to 2 months or up to 1 month). In some instances, the human patient may have undergone a prior autologous hematopoietic stem cell transplantation (HSCT) and showed no response to such (failed) or have progressed or relapsed after achieving some response. In other instances, the human patient may not be eligible for prior autologous HSCT. See, e.g., Table 12 in Example 12 below, each of the patient populations listed therein is within the scope of the present disclosure.

A human patient may be screened to determine whether the patient is eligible to undergo a conditioning regimen (lymphodepleting treatment) and/or an allogeneic anti-CD19 CAR-T cell therapy as disclosed herein. For example, a human patient who is eligible for lymphodepletion treatment does not show one or more of the following features: (a) significant worsening of clinical status, (b) requirement for supplemental oxygen to maintain a saturation level of greater than 90%, (c) uncontrolled cardiac arrhythmia, (d) hypotension requiring vasopressor support, (e) active infection, and (f) grade ≥2 acute neurological toxicity. In another example, a human patient who is eligible for a treatment regimen does not show one or more of the following features: (a) active uncontrolled infection, (b) worsening of clinical status compared to the clinical status prior to lymphodepletion treatment, and (c) grade ≥2 acute neurological toxicity. See also Example 12 below.

A human patient may be screened and excluded from the conditioning regimen and/or treatment regimen based on such screening results. For example, a human patient may be excluded from a conditioning regimen and/or the allogeneic anti-CD19 CAR-T cell therapy, if the patient meets one or more of the following exclusion criteria: (a) has an Eastern Cooperative Oncology Group (ECOG) performance status 0 or 1; (b) adequate renal, liver, cardiac, and/or pulmonary function; (c) free of prior gene therapy or modified cell therapy; (d) free of prior treatment comprising an anti-CD19 antibody; (e) free of prior allogeneic HSCT; (f) free of detectable malignant cells from cerebrospinal fluid; (g) free of brain metastases; (h) free of prior central nervous system disorders; (i) free of unstable angina, arrhythmia, and/or myocardial infarction; (j) free of uncontrolled infection; (k) free of immunodeficiency disorders or autoimmune disorders that require immunosuppressive therapy; and (1) free of infection by human immunodeficiency virus, hepatitis B virus, or hepatitis C virus. See also Example 12 below.

In some embodiments, the human patient for treatment by the methods disclosed herein may meet the inclusion and exclusion criteria provided in Example 12 below.

(ii) Conditioning Regimen (Lymphodepleting Therapy)

Any human patients suitable for the treatment methods disclosed herein may receive a lymphodepleting therapy to reduce or deplete the endogenous lymphocyte of the subject.

Lymphodepletion refers to the destruction of endogenous lymphocytes and/or T cells, which is commonly used prior to immunotransplantation and immunotherapy. Lymphodepletion can be achieved by irradiation and/or chemotherapy. A “lymphodepleting agent” can be any molecule capable of reducing, depleting, or eliminating endogenous lymphocytes and/or T cells when administered to a subject. In some embodiments, the lymphodepleting agents are administered in an amount effective in reducing the number of lymphocytes by at least 10%, 20%, 30%, 40%, 50%, 60%, 70%, 80%, 90%, 95%, 96%, 96%, 97%, 98%, or at least 99% as compared to the number of lymphocytes prior to administration of the agents. In some embodiments, the lymphodepleting agents are administered in an amount effective in reducing the number of lymphocytes such that the number of lymphocytes in the subject is below the limits of detection. In some embodiments, the subject is administered at least one (e.g., 2, 3, 4, 5 or more) lymphodepleting agents.

In some embodiments, the lymphodepleting agents are cytotoxic agents that specifically kill lymphocytes. Examples of lymphodepleting agents include, without limitation, fludarabine, cyclophosphamide, bendamustin, 5-fluorouracil, gemcitabine, methotrexate, dacarbazine, melphalan, doxorubicin, vinblastine, cisplatin, oxaliplatin, paclitaxel, docetaxel, irinotecan, etopside phosphate, mitoxantrone, cladribine, denileukin diftitox, or DAB-IL2. In some instances, the lymphodepleting agent may be accompanied with low-dose irradiation. The lymphodepletion effect of the conditioning regimen can be monitored via routine practice.

In some embodiments, the method described herein involves a conditioning regimen that comprises one or more lymphodepleting agents, for example, fludarabine and cyclophosphamide. A human patient to be treated by the method described herein may receive multiple doses of the one or more lymphodepleting agents for a suitable period (e.g., 1-5 days) in the conditioning stage. The patient may receive one or more of the lymphodepleting agents once per day during the lymphodepleting period. In one example, the human patient receives fludarabine at about 20-50 mg/m² (e.g., 30 mg/m²) per day for 2-4 days (e.g., 3 days) and cyclophosphamide at about 500-750 mg/m² (e.g., 500 or 750 mg/m²) per day for 2-4 days (e.g., 3 days). In specific examples, the human patient may receive fludarabine at about 30 mg/m² and cyclophosphamide at about 500 mg/m² per day for three days.

The human patient may then be administered any of the anti-CD19 CAR T cells such as CTX112 cells within a suitable period after the lymphodepleting therapy as disclosed herein. For example, a human patient may be subject to one or more lymphodepleting agent about 2-7 days (e.g., for example, 2, 3, 4, 5, 6, 7 days) before administration of the anti-CD19 CAR+ T cells (e.g., CTX112 cells). In some instances, a human patient is administered the anti-CD19 CAR+ T cells (e.g., CTX112 cells) within about 4-5 days after the lymphodepleting therapy.

Since the allogeneic anti-CD19 CAR-T cells such as CTX112 cells can be prepared in advance and may be stored at the treatment site, the lymphodepleting therapy as disclosed herein may be applied to a human patient having a B cell malignancy within a short time window (e.g., within 2 weeks) after the human patient is identified as suitable for the allogeneic anti-CD19 CAR-T cell therapy disclosed herein. For example, the first dose of the lymphodepleting therapy (e.g., fludarabine at about 30 mg/m² and cyclophosphamide at about 500 mg/m²) may be administered to the human patient within two weeks (e.g., within 10 days, within 9 days, within 8 days, within 7 days, within 6 days, within 5 days, within 4 days, within 3 days, within two days, or less) after the human patient is identified as suitable for the allogeneic anti-CD19 CAR-T cell therapy. In some examples, the lymphodepleting therapy may be performed to the human patient within 24-72 hours (e.g., within 24 hours) after the human patient is identified as suitable for the treatment. The patient can then be administered the CAR-T cells within 2-7 days (e.g., for example, 2, 3, 4, 5, 6, or 7 days) after the lymphodepleting treatment. This allows for timely treatment of the human patient with the allogeneic anti-CD19 CAR-T cells disclosed herein such as CTX112 cells after disease diagnosis and/or patient identification without delay (e.g., delay due to preparation of the therapeutic cells). In certain instances, a patient may receive the treatment during inpatient hospital care. In certain instances, a patient may receive the treatment in outpatient care.

Prior to any of the lymphodepletion steps, a human patient may be screened for one or more features to determine whether the patient is eligible for lymphodepletion treatment. For example, prior to lymphodepletion, a human patient eligible for lymphodepletion treatment does not show one or more of the following features: (a) significant worsening of clinical status, (b) requirement for supplemental oxygen to maintain a saturation level of greater than 90%, (c) uncontrolled cardiac arrhythmia, (d) hypotension requiring vasopressor support, (e) active infection, and (f) grade ≥2 acute neurological toxicity.

Following lymphodepletion, a human patient may be screened for one or more features to determine whether the patient is eligible for treatment with anti-CD19 CAR T cells such as the CTX112 cells. For example, prior to anti-CD19 CAR T cell treatment and after lymphodepletion treatment, a human patient eligible for anti-CD19 CAR T cells treatment does not show one or more of the following features: (a) active uncontrolled infection, (b) worsening of clinical status compared to the clinical status prior to lymphodepletion treatment, and (c) grade ≥2 acute neurological toxicity.

(iii) Administration of Anti-CD19 CAR T Cells

Administering anti-CD19 CAR T cells may include placement (e.g., transplantation) of a genetically engineered T cell population as disclosed herein (e.g., the CTX112 cells) into a human patient as also disclosed herein by a method or route that results in at least partial localization of the genetically engineered T cell population at a desired site, such as a tumor site, such that a desired effect(s) can be produced. The genetically engineered T cell population can be administered by any appropriate route that results in delivery to a desired location in the subject where at least a portion of the implanted cells or components of the cells remain viable. The period of viability of the cells after administration to a subject can be as short as a few hours, e.g., twenty-four hours, to a few days, to several weeks or months, to as long as several years, or even the life time of the subject, i.e., long-term engraftment. In certain instances, a patient may receive the genetically engineered T cell population (e.g., CTX112 cells) during inpatient hospital care. In certain instances, a patient may receive genetically engineered T cell population (e.g., CTX112 cells) in outpatient care.

For example, an effective amount of the genetically engineered T cell population can be administered via a systemic route of administration, such as an intraperitoneal or intravenous route.

In some embodiments, the genetically engineered T cell population is administered systemically, which refers to the administration of a population of cells other than directly into a target site, tissue, or organ, such that it enters, instead, the subject's circulatory system and, thus, is subject to metabolism and other like processes. Suitable modes of administration include injection, infusion, instillation, or ingestion. Injection includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some embodiments, the route is intravenous.

An effective amount refers to the amount of a genetically engineered T cell population needed to prevent or alleviate at least one or more signs or symptoms of a medical condition (e.g., a B cell malignancy), and relates to a sufficient amount of a genetically engineered T cell population to provide the desired effect, e.g., to treat a subject having a medical condition. An effective amount also includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

An effective amount of a genetically engineered T cell population may comprise about 1×10⁷ anti-CD19 CAR+ cells to about 6.0×10⁸ anti-CD19 CAR+ cells, e.g., about 2.0×10⁸ to about 6.0×10⁸ cells or about 3.0×10⁷ cells to about 6.0×10⁸ cells that express a CAR that binds CD19 (CAR+ cells), for example, CAR+ CTX112 cells. In some embodiments, the effective amount of the anti-CD19 CAR+ T cells may range from about 3×10⁷ to about 1.0×10⁸ CAR+ T cells, about 3.0×10⁷ to about 2.5×10⁸ CAR+ T cells, about 3×10⁷ to about 3.0×10⁸ CAR+ T cells, about 3.0×10⁷ to about 4.5×10⁸ CAR+ T cells, or about 3×10⁷ to about 1.0×10⁸ CAR+ T cells. In other embodiments, the effective amount of the anti-CD19 CAR+ T cells may range from about 1×10⁸ to about 2×10⁸, from about 1×10⁸ to about 3×10⁸ CAR+ T cells, from about 1×10⁸ to about 4.5×10⁸, or from about 1×10⁸ to about 6.0×10⁸ CAR+ T cells. In some embodiments, the effective amount of the anti-CD19 CAR+ T cells may be from about 2.0×10⁸ to about 3×10⁸ CAR+ T cells, from about 3.0×10⁸ to about 4.5×10⁸ CAR+ T cells, or from about 4.5×10⁸ to about 6×10⁸ CAR+ T cells. In yet other embodiments, the effective amount of the anti-CD19 CAR+ T cells may range from about 3×10⁸ to about 4.5×10⁸ CAR+ T cells or about 3×10⁸ to about 6×10⁸ CAR+ T cells. In some embodiments, the effective amount of the anti-CD19 CAR+ T cells may range from about 4.5×10⁸ to about 6×10⁸ CAR+ T cells.

In some embodiments, an effective amount of a genetically engineered T cell population may comprise a dose of the genetically engineered T cell population, e.g., a dose comprising about 1×10⁷ CTX112 cells to about 6.0×10⁸ CTX112 cells. In some embodiments, the effective amount of the CAR+ CTX112 cells may range from about 3×10⁷ to about 6.0×10⁸ CAR+ CTX112 cells, about 3.0×10⁷ to about 3.0×10⁸ CAR+ CTX112 cells, or about 3×10⁷ to about 1.0×10⁸ CAR+ CTX112 cells. In other embodiments, the effective amount of the anti-CD19 CAR+ CTX112 cells may range from about 1×10⁸ to about 3×10⁸ CAR+ CTX112 cells, about 1×10⁸ to about 4.5×10⁸ CAR+ CTX112 cells, or about 1×10⁸ to about 6×10⁸ CAR+ CTX112 cells. In yet other embodiments, the effective amount of the anti-CD19 CAR+ CTX112 cells may range from about 3×10⁸ to about 4.5×10⁸ CAR+ CTX112 cells or about 3×10⁸ to about 6×10⁸ CAR+ CTX112 cells. In some embodiments, the effective amount of the anti-CD19 CAR+ CTX112 cells may range from about 4.5×10⁸ to about 6×10⁸ CAR+ CTX112 cells.

In some examples, an effective amount of a genetically engineered T cell population may be about 1×10⁷ CAR⁺ CTX112 cells. In some examples, an effective amount of a genetically engineered T cell population may be about 3×10⁷ CAR⁺ CTX112 cells. In some examples, an effective amount of a genetically engineered T cell population may be about 1×10⁸ CAR⁺ CTX112 cells. In some examples, an effective amount of a genetically engineered T cell population may be about 2×10⁸ CAR⁺ CTX112 cells. In some examples, an effective amount of a genetically engineered T cell population may be about 3×10⁸ CAR⁺ CTX112 cells. In some examples, an effective amount of a genetically engineered T cell population may be about 4.5×10⁸ CAR⁺ CTX112 cells. In some examples, an effective amount of a genetically engineered T cell population may be about 6×10⁸ CAR⁺ CTX112 cells.

In some instances, the anti-CD19 CAR-T cells disclosed herein such as the CTX112 cells can be non-autologous (“non-self,” e.g., allogeneic, syngeneic or xenogeneic) to the subject. “Allogeneic” means that the anti-CD19 CAR-T cells are not derived from the subject who receives the treatment but from different individuals (donors) of the same species as the subject. A donor is an individual who is not the subject being treated. A donor is an individual who is not the patient. In some embodiments, a donor is an individual who does not have or is not suspected of having the cancer being treated. In some embodiments, multiple donors, e.g., two or more donors, are used. In some embodiments, the anti-CD19 CAR-T cell population being administered according to the methods described herein comprises allogeneic T cells obtained from one or more donors, for example, one or more healthy human donors.

An effective amount refers to the amount of the anti-CD19 CAR-T cells disclosed herein needed to prevent or alleviate at least one or more signs or symptoms of a medical condition (e.g., cancer), and relates to a sufficient amount of a composition to provide the desired effect, e.g., to treat a subject having a medical condition. An effective amount also includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

The efficacy of a treatment using the anti-CD19 CAR-T cells disclosed herein can be determined by the skilled clinician. A treatment is considered “effective”, if any one or all of the signs or symptoms of, as but one example, levels of functional target are altered in a beneficial manner (e.g., increased by at least 10%), or other clinically accepted symptoms or markers of disease (e.g., cancer) are improved or ameliorated. Efficacy can also be measured by failure of a subject to worsen as assessed by hospitalization or need for medical interventions (e.g., progression of the disease is halted or at least slowed). Methods of measuring these indicators are known to those of skill in the art and/or described herein. Treatment includes any treatment of a disease in subject and includes: (1) inhibiting the disease, e.g., arresting, or slowing the progression of symptoms; or (2) relieving the disease, e.g., causing regression of symptoms; and (3) preventing or reducing the likelihood of the development of symptoms.

When needed, a second dose (or, in some instances, more doses) of any of the genetically engineered anti-CD19 CAR-T cells such as CTX112 cells may be given to the same patient who received a first dose of the anti-CD19 CAR-T cells. Patients eligible for redosing may show partial response (PR) or a better response (e.g., complete response) resulting from the first dose. The subsequent dose or additional doses may be identical to the first dose. Alternatively, the second dose or additional doses may be different from the first dose, higher or lower. In some examples, the second dose may range from about 3.0×10⁷ to about 6.0×10⁸ CAR+ T cells, for example, about 3.0×10⁷ CAR+ cells, about 1.0×10⁸ CAR+ cells, about 2.0×10⁸ CAR+ cells, about 3.0×10⁸ CAR+ cells, about 4.5×10⁸ CAR+ cells, or about 6×10⁸ CAR+ T cells.

In some examples, a second dose of the anti-CD19 CAR T cells (e.g., CTX112) or any of the subsequent doses may be preceded by a lymphodepletion treatment, e.g., following the same lymphodepletion conditions (e.g., the same lymphodepletion agents at the same doses and the same timeframe relative to the anti-CD19 CAR-T cell therapy). A human patient eligible for a second dose (or any additional doses) may be confirmed that tumor remains CD19+ at relapse (e.g., by flow cytometry or IHC). Such a patient may not exhibit prior dose-limiting-toxicity (DLT) after the first dose, may not show prior grade ≥3 CRS without resolution to grade ≤2 within 72 hours following the first dose, may not show prior GvHD following the first dose, and/or may not show grade 4 ICANS following the first dose and any grade 1, 2, or 3 ICANS must have resolved more than 14 days prior to a subsequent dose. Further, the human patient may meet the inclusion and exclusion criteria provided in Example 12 below, where applicable. The human patient may also meet the criteria for LD chemotherapy as provided in Example 12.

Following each dosing of anti-CD19 CAR T cells, a human patient may be monitored for acute toxicities such as tumor lysis syndrome (TLS), cytokine release syndrome (CRS), neurotoxicity such as immune effector cell-associated neurotoxicity syndrome (ICANS) or viral encephalitis, B cell aplasia, hemophagocytic lymphohistiocytosis (HLH), cytopenia, graft-versus-host disease (GvHD), hypertension, renal insufficiency, or a combination thereof.

When a human patient exhibits one or more symptoms of acute toxicity, the human patient may be subjected to toxicity management. Treatments for patients exhibiting one or more symptoms of acute toxicity are known in the art. For example, a human patient exhibiting a symptom of CRS (e.g., cardiac, respiratory, and/or neurological abnormalities) may be administered an anti-cytokine therapy. In addition, a human patient that does not exhibit a symptom of CRS may be administered an anti-cytokine therapy to promote proliferation of anti-CTX112 CAR T cells. See, e.g., Example 12 below.

Alternatively, or in addition to, when a human patient exhibits one or more symptoms of acute toxicity, treatment of the human patient may be terminated. Patient treatment may also be terminated if the patient exhibits one or more signs of an adverse event (AE), e.g., the patient has an abnormal laboratory finding and/or the patient shows signs of disease progression.

Combination therapies are also encompassed by the present disclosure. For example, the therapeutic T cells disclosed herein may be co-used with other therapeutic agents, for treating the same indication, or for enhancing efficacy of the therapeutic T cells and/or reducing side effects of the therapeutic T cells.

The allogeneic anti-CD19 CAR T cell therapy (e.g., involving the CTX112 cells) described herein may also be used in combination therapies. For example, anti-CD19 CAR T cells treatment methods described herein may be co-used with other therapeutic agents, for treating a B cell malignancy, or for enhancing efficacy of the genetically engineered T cell population and/or reducing side effects of the genetically engineered T cell population.

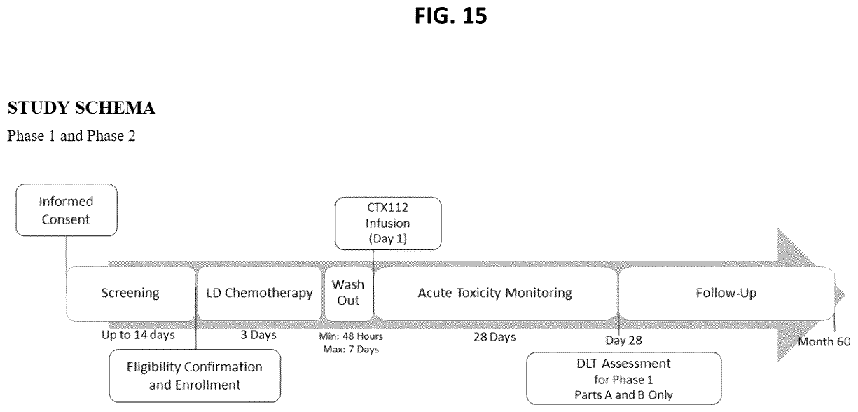

A human patient having a CD19+ B cell malignancy can be treated by any of the treatment methods disclosed herein, using the anti-CD19 CAR-T cells (e.g., CTX112). One example of the treatment regimen is provided in FIG. 15. For example, a human patient having a relapsed/refractory B-cell malignancy as disclosed herein may be identified for the treatment disclosed herein. Such a human patient may have Follicular lymphoma (FL) grade 1-3a, Mantle cell lymphoma (MCL), Marginal zone lymphoma (MZL), Chronic lymphocytic leukemia (CLL)/small lymphocytic lymphoma (SLL), or a large B cell lymphoma (LBLC) including diffuse large B cell lymphoma (DLBCL) not otherwise specified (NOS), and high-grade B cell lymphoma with MYC and BCL2 and/or BCL6 rearrangements, primary mediastinal large B cell lymphoma (PMBCL; Phase 2 only), transformed FL and grade 3b FL, transformed MCL, or transformed MZL. The human patient may meet the inclusion and exclusion criteria provided in Example 12 below. The human patient may receive an LD chemotherapy comprising co-administration of fludarabine 30 mg/m² and cyclophosphamide 500 mg/m² IV daily for 3 days. In some instances, both agents may be started on the same day and administered for 3 consecutive days and completed at least 48 hours (but no more than 7 days) prior to CTX112 infusion. The anti-CD19 CAR-T cells (e.g., CTX112) is administered to the human patient at a dose of at least 3×10⁷ CAR+ T cells via intravenous infusion, e.g., 3×10⁷ CAR+ T cells, 1.0×10⁸ CAR+ T cells, 2×10⁸ CAR+ T cells, 3×10⁸ CAR+ T cells, 4.5×10⁸ CAR+ T cells, or 6×10⁸ CAR+ T cells.

The human patient is then monitored for development of acute toxicity within about 28-day after the CAR-T cell infusion. A suitable toxicity management approach (e.g., those disclosed in Example 12 below) may be performed when needed.

### IV. Kit for Allogeneic CAR-T Cell Therapy of B Cell Malignancies

The present disclosure also provides kits for use of a population of anti-CD19 CAR T cells such as CTX112 cells as described herein in methods for treating a B cell malignancy. Such kits may include one or more containers comprising a first pharmaceutical composition that comprises one or more lymphodepleting agents, and a second pharmaceutical composition that comprises any nucleic acid or population of genetically engineered T cells (e.g., those described herein), and a pharmaceutically acceptable carrier. Kits comprising the genetically engineered CAR-T cells as disclosed herein, such at the CTX112 cells, may be stored and inventoried at the site of care, allowing for rapid treatment of human patients following diagnosis.

In some embodiments, the kit can comprise instructions for use in any of the methods described herein. The included instructions can comprise a description of administration of the first and/or second pharmaceutical compositions to a subject to achieve the intended activity in a human patient. The kit may further comprise a description of selecting a human patient suitable for treatment based on identifying whether the human patient is in need of the treatment. In some embodiments, the instructions comprise a description of administering the first and second pharmaceutical compositions to a human patient who is in need of the treatment.

The instructions relating to the use of a population of anti-CD19 CAR T cells such as CTX112 T cells described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the population of genetically engineered T cells is used for treating, delaying the onset, and/or alleviating a T cell or B cell malignancy in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port. At least one active agent in the pharmaceutical composition is a population of the anti-CD19 CAR-T cells such as the CTX112 T cells as disclosed herein.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### Sequence Tables

TABLE 1
sRNA Sequences and Target Sequences
gRNA Sequences

| Name | Unmodified Sequence | Modified Sequence |
|---|---|---|
| TRAC sgRNA | AGAGCAACAGUGCUGUG | A*G*A*GCAACAGUGCUG |
| | GCCguuuuagagcuagaaauagca | UGGCCguuuuagagcuagaaaua |
| | aguuaaaauaaggcuaguccguuauc | gcaaguuaaaauaaggcuaguccguu |
| | aacuugaaaaaguggcaccgagucgg | aucaacuugaaaaaguggcaccgagu |
| | ugcUUUU | cggugcU*U*U*U |
| | (SEQ ID NO: 1) | (SEQ ID NO: 2) |
| TRAC sgRNA spacer | AGAGCAACAGUGCUGUG | A*G*A*GCAACAGUGCUG |
| | GCC (SEQ ID NO: 3) | UGGCC (SEQ ID NO: 4) |
| β2M sgRNA | GCUACUCUCUCUUUCUG | G*C*U*ACUCUCUCUUUC |
| | GCCguuuuagagcuagaaauagca | UGGCCguuuuagagcuagaaaua |
| | aguuaaaauaaggcuaguccguuauc | gcaaguuaaaauaaggcuaguccguu |
| | aacuugaaaaaguggcaccgagucgg | aucaacuugaaaaaguggcaccgagu |
| | ugcUUUU (SEQ ID NO: 5) | cggugcU*U*U*U (SEQ ID |
| | | NO: 6) |
| β2M sgRNA spacer | GCUACUCUCUCUUUCUG | G*C*U*ACUCUCUCUUUC |
| | GCC (SEQ ID NO: 7) | UGGCC (SEQ ID NO: 8) |
| Reg1 sgRNA | ACGACGCGUGGGUGGCA | A*C*G*ACGCGUGGGUGG |
| | AGCguuuuagagcuagaaauagca | CAAGCguuuuagagcuagaaaua |
| | aguuaaaauaaggcuaguccguuauc | gcaaguuaaaauaaggcuaguccguu |
| | aacuugaaaaaguggcaccgagucgg | aucaacuugaaaaaguggcaccgagu |
| | ugcUUUU (SEQ ID NO: 9) | cggugcU*U*U*U (SEQ ID |
| | | NO: 10) |
| Reg1 sgRNA spacer | ACGACGCGUGGGUGGCA | A*C*G*ACGCGUGGGUGG |
| | AGC (SEQ ID NO: 11) | CAAGC (SEQ ID NO: 12) |
| TGFBRII sgRNA | CCCCUACCAUGACUUUA | C*C*C*CUACCAUGACUU |
| | UUCguuuuagagcuagaaauagca | UAUUCguuuuagagcuagaaaua |
| | aguuaaaauaaggcuaguccguuauc | gcaaguuaaaauaaggcuaguccguu |
| | aacuugaaaaaguggcaccgagucgg | aucaacuugaaaaaguggcaccgagu |
| | ugcUUUU (SEQ ID NO: 13) | cggugcU*U*U*U (SEQ ID |
| | | NO: 14) |
| TGFBRII sgRNA spacer | CCCCUACCAUGACUUUA | C*C*C*CUACCAUGACUU |
| | UUC (SEQ ID NO: 15) | UAUUC (SEQ ID NO: 16) |

Target Sequences

| | | Target Sequence-Without |
|---|---|---|
| Guide Name | Target Sequence (PAM) | PAM |
| TRAC sgRNA | AGAGCAACAGTGCTGTG | AGAGCAACAGTGCTGTG |
| | GCC(TGG) (SEQ ID NO: 17) | GCC (SEQ ID NO: 18) |
| β2M sgRNA | GCTACTCTCTCTTTCTGG | GCTACTCTCTCTTTCTGG |
| | CC(TGG) (SEQ ID NO: 19) | CC (SEQ ID NO: 20) |
| Reg1 sgRNA | ACGACGCGTGGGTGGCA | ACGACGCGTGGGTGGCA |
| | AGC(GGG) (SEQ ID NO: 21) | AGC (SEQ ID NO: 22) |
| TGFBRII sgRNA | CCCCTACCATGACTTTAT | CCCCTACCATGACTTTAT |
| | TC(TGG) (SEQ ID NO: 23) | TC (SEQ ID NO: 24) |

Exemplary sgRNA Formula
nnnnnnnnnnnnnnnnnnnnguuuuagagcuagaaauagcaaguuaaaauaaggcuaguccguuaucaacuugaaaaag
uggcaccgagucggugcuuuu (SEQ ID NO: 25)
nnnnnnnnnnnnnnnnnnnnguuuuagagcuagaaauagcaaguuaaaauaaggcuaguccguuaucaacuugaaaaag
uggcaccgagucggugc (SEQ ID NO: 26)
n₍₁₇₋₃₀₎guuuuagagcuagaaauagcaaguuaaaauaaggcuaguccguuaucaacuug
aaaaaguggcaccgagucggugcu₍₁₋₈₎ (SEQ ID NO: 27)
Exemplary Cas9 sequence (spCas9)
MDKKYSIGLDIGTNSVGWAVITDEYKVPSKKFKVLGNTDRHSIKKNLIGALLFDSGET
AEATRLKRTARRRYTRRKNRICYLQEIFSNEMAKVDDSFFHRLEESFLVEEDKKHERH
PIFGNIVDEVAYHEKYPTIYHLRKKLVDSTDKADLRLIYLALAHMIKFRGHFLIEGDLN
PDNSDVDKLFIQLVQTYNQLFEENPINASGVDAKAILSARLSKSRRLENLIAQLPGEKK
NGLFGNLIALSLGLTPNFKSNFDLAEDAKLQLSKDTYDDDLDNLLAQIGDQYADLFLA
AKNLSDAILLSDILRVNTEITKAPLSASMIKRYDEHHQDLTLLKALVRQQLPEKYKEIFF
DQSKNGYAGYIDGGASQEEFYKFIKPILEKMDGTEELLVKLNREDLLRKQRTFDNGSIP
HQIHLGELHAILRRQEDFYPFLKDNREKIEKILTFRIPYYVGPLARGNSRFAWMTRKSE
ETITPWNFEEVVDKGASAQSFIERMTNFDKNLPNEKVLPKHSLLYEYFTVYNELTKVK
YVTEGMRKPAFLSGEQKKAIVDLLFKTNRKVTVKQLKEDYFKKIECFDSVEISGVEDR
FNASLGTYHDLLKIIKDKDFLDNEENEDILEDIVLTLTLFEDREMIEERLKTYAHLFDDK
VMKQLKRRRYTGWGRLSRKLINGIRDKQSGKTILDFLKSDGFANRNFMQLIHDDSLTF
KEDIQKAQVSGQGDSLHEHIANLAGSPAIKKGILQTVKVVDELVKVMGRHKPENIVIE
MARENQTTQKGQKNSRERMKRIEEGIKELGSQILKEHPVENTQLQNEKLYLYYLONG
RDMYVDQELDINRLSDYDVDHIVPQSFLKDDSIDNKVLTRSDKNRGKSDNVPSEEVV
KKMKNYWRQLLNAKLITQRKFDNLTKAERGGLSELDKAGFIKRQLVETRQITKHVAQ
ILDSRMNTKYDENDKLIREVKVITLKSKLVSDFRKDFQFYKVREINNYHHAHDAYLNA
VVGTALIKKYPKLESEFVYGDYKVYDVRKMIAKSEQEIGKATAKYFFYSNIMNFFKTE
ITLANGEIRKRPLIETNGETGEIVWDKGRDFATVRKVLSMPQVNIVKKTEVQTGGFSKE
SILPKRNSDKLIARKKDWDPKKYGGFDSPTVAYSVLVVAKVEKGKSKKLKSVKELLGI
TIMERSSFEKNPIDFLEAKGYKEVKKDLIIKLPKYSLFELENGRKRMLASAGELQKGNE
LALPSKYVNFLYLASHYEKLKGSPEDNEQKQLFVEQHKHYLDEIIEQISEFSKRVILAD
ANLDKVLSAYNKHRDKPIREQAENIIHLFTLTNLGAPAAFKYFDTTIDRKRYTSTKEVL
DATLIHQSITGLYETRIDLSQLGGD (SEQ ID NO: 28)
*: 2′-O-methyl phosphorothioate residue
“n” refers to the spacer sequence at the 5′ end

TABLE 2
Edited β2M Gene Sequence.

| | Sequence (Deletions indicated by dashes (-); | SEQ ID |
|---|---|---|
| Description | insertions indicated by bold) | NO: |
| β2M gene-edit | CGTGGCCTTAGCTGTGCTCGCGCTACTCTCTCTTTCT- | 29 |
| | GCCTGGAGGCTATCCAGCGTGAGTCTCTCCTACCCTCCCGCT | |
| β2M gene-edit | CGTGGCCTTAGCTGTGCTCGCGCTACTCTCTCTTTC-- | 30 |
| | GCCTGGAGGCTATCCAGCGTGAGTCTCTCCTACCCTCCCGCT | |
| β2M gene-edit | CGTGGCCTTAGCTGTGCTCGCGCTACTCTCTCTTT----- | 31 |
| | CTGGAGGCTATCCAGCGTGAGTCTCTCCTACCCTCCCGCT | |
| β2M gene-edit | CGTGGCCTTAGCTGTGCTCGCGCTACTCTCTCTTTCTGGATAGCCTG | 32 |
| | GAGGCTATCCAGCGTGAGTCTCTCCTACCCTCCCGCT | |
| β2M gene-edit | CGTGGCCTTAGCTGTGCTCGC------------------------- | 33 |
| | GCTATCCAGCGTGAGTCTCTCCTACCCTCCCGCT | |
| β2M gene-edit | CGTGGCCTTAGCTGTGCTCGCGCTACTCTCTCTTTCTGTGGCCTGGA | 34 |
| | GGCTATCCAGCGTGAGTCTCTCCTACCCTCCCGCT | |

TABLE 3
Exemplary Nucleotide Sequences in Disrupted TGFBRII Gene

| Description | Gene Edited Sequence^{b} | SEQ ID NO |
|---|---|---|
| TGFBRII Gene-edit | CATGA-------CTGGAAGA | 35 |
| TGFBRII Gene-edit | CATGAC----TTCTGGAAGA | 36 |
| TGFBRII Gene-edit | CATGACT---TTCTGGAAGA | 37 |
| TGFBRII Gene-edit | CATGACTTTATTTCTGGAAGA | 38 |
| TGFBRII Gene-edit | CATGACTTTAATTCTGGAAGA | 39 |
| TGFBRII Gene-edit | CA-----------TGGAAGA | |
| TGFBRII Gene-edit | CATGACTT--TTCTGGAAGA | 40 |
| TGFBRII Gene-edit | CAT------------GAAGA | |
| TGFBRII Gene-edit | C------------------A | |
| TGFBRII Gene-edit | -------------------- | |
| TGFBRII Gene-edit | CATGA--------------- | |
| TGFBRII Gene-edit | CAT---------------GA | |
| TGFBRII Gene-edit | CA---------TCTGGAAGA | 41 |
| TGFBRII Gene-edit | CATGACTTT-TTCTGGAAGA | 42 |
| TGFBRII Gene-edit | CATGACTTTA-TCTGGAAGA | 43 |
| TGFBRII Gene-edit | CATGACTTT-------AAGA | 44 |
| TGFBRII Gene-edit | ----------TTCTGGAAGA | 45 |
| TGFBRII Gene-edit | CATGACTTTA--CTGGAAGA | 46 |

TABLE 4
Exemplary Nucleotide Sequences in Disrupted Reg1
Gene

| Description | Gene Edited Sequence^{b} | SEQ ID NO |
|---|---|---|
| Reg1 Gene-edit | GTGGGTGGCAAAGCGGGTGGT | 47 |
| Reg1 Gene-edit | GT-----------GGGTGGT | |
| Reg1 Gene-edit | -----------GCGGGTGGT | |
| Reg1 Gene-edit | GTGGGTGGC-AGCGGGTGGT | 48 |
| Reg1 Gene-edit | ---------------GTGGT | |
| Reg1 Gene-edit | GTG--------------GGT | |
| Reg1 Gene-edit | ------------CGGGTGGT | |
| Reg1 Gene-edit | -------------------- | |
| Reg1 Gene-edit | GTGGGTGGC----------- | |
| Reg1 Gene-edit | GTGGGTGGCATAGCGGGTGGT | 49 |
| Reg1 Gene-edit | GTGGGTG------------- | |
| Reg1 Gene-edit | GTGG---------------- | |
| Reg1 Gene-edit | GTGGGTGG--AGCGGGTGGT | 50 |

TABLE 5
Chimeric Antigen Receptor Sequences

| | | SEQ ID |
|---|---|---|
| Description | Sequence | NO |
| signal peptide | MLLLVTSLLLCELPHPAFLLTP | 51 |
| signal peptide | MALPVTALLLPLALLLHAARP | 52 |
| CD8a transmembrane | TYTWAPLAGTCGVLLLSLVTTLY | 53 |
| domain | | |
| 4-1BB nucleotide | AAACGGGGCAGAAAGAAACTCCTGTATATATTCAAACAACCATTT | 54 |
| sequence | ATGAGACCAGTACAAACTACTCAAGAGGAAGATGGCTGTAGCTGC | |
| | CGATTTCCAGAAGAAGAAGAAGGAGGATGTGAACTG | |
| 4-1BB amino acid | KRGRKKLLYTFKQPFMRPVQTTQEEDGCSCRFPEEEEGGCEL | 55 |
| sequence | | |
| CD28 nucleotide | TCAAAGCGGAGTAGGTTGTTGCATTCCGATTACATGAATATGACT | 56 |
| sequence | CCTCGCCGGCCTGGGCCGACAAGAAAACATTACCAACCCTATGCC | |
| | CCCCCACGAGACTTCGCTGCGTACAGGTCC | |
| CD28 amino acid | SKRSRLLHSDYMNMTPRRPGPTRKHYQPYAPPRDFAAYRS | 57 |
| sequence | | |
| CD3-zeta nucleotide | CGAGTGAAGTTTTCCCGAAGCGCAGACGCTCCGGCATATCAGCAA | 58 |
| sequence | GGACAGAATCAGCTGTATAACGAACTGAATTTGGGACGCCGCGAG | |
| | GAGTATGACGTGCTTGATAAACGCCGGGGGAGAGACCCGGAAATG | |
| | GGGGGTAAACCCCGAAGAAAGAATCCCCAAGAAGGACTCTACAAT | |
| | GAACTCCAGAAGGATAAGATGGCGGAGGCCTACTCAGAAATAGGT | |
| | ATGAAGGGCGAACGACGACGGGGAAAAGGTCACGATGGCCTCTAC | |
| | CAAGGGTTGAGTACGGCAACCAAAGATACGTACGATGCACTGCAT | |
| | ATGCAGGCCCTGCCTCCCAGA | |
| CD3-zeta amino acid | RVKFSRSADAPAYQQGQNQLYNELNLGRREEYDVLDKRRGRDPEM | 59 |
| sequence | GGKPRRKNPQEGLYNELQKDKMAEAYSETGMKGERRRGKGHDGLY | |
| | QGLSTATKDTYDALHMQALPPR | |
| anti-CD19 VL CDR1 | RASQDTSKYLN | 60 |
| (Kabat) | | |
| anti-CD19 VL CDR2 | HTSRLHS | 61 |
| (Kabat) | | |
| anti-CD19 VL CDR3 | QQGNTLPYT | 62 |
| (Kabat) | | |
| anti-CD19 VH | DYGVS | 63 |
| CDR1 (Kabat) | | |
| anti-CD19 VH | VTWGSETTYYNSALKS | 64 |
| CDR2 (Kabat) | | |
| anti-CD19 VH | HYYYGGSYAMDY | 65 |
| CDR3 (Kabat) | | |
| anti-CD19 VL CDR1 | RASQDTSKYLN | 66 |
| (Chothia) | | |
| anti-CD19 VL CDR2 | HTSRLHS | 67 |
| (Chothia) | | |
| anti-CD19 VL CDR3 | QQGNTLPYT | 68 |
| (Chothia) | | |
| anti-CD19 VH | GVSLPDY | 69 |
| CDR1 (Chothia) | | |
| anti-CD19 VH | WGSET | 70 |
| CDR2 (Chothia) | | |
| anti-CD19 VH | HYYYGGSYAMDY | 71 |
| CDR3 (Chothia) | | |
| Anti-CD19 CAR | ATGCTTCTTTTGGTTACGTCTCTGTTGCTTTGCGAACTTCCTCAT | 72 |
| FMC63-28Z | CCAGCGTTCTTGCTGATCCCCGATATTCAGATGACTCAGACCACC | |
| (FMC63-CD8[tm]- | AGTAGCTTGTCTGCCTCACTGGGAGACCGAGTAACAATCTCCTGC | |
| CD28[co-stimulatory | AGGGCAAGTCAAGACATTAGCAAATACCTCAATTGGTACCAGCAG | |
| domain]-CD3z) | AAGCCCGACGGAACGGTAAAACTCCTCATCTATCATACGTCAAGG | |
| | TTGCATTCCGGAGTACCGTCACGATTTTCAGGTTCTGGGAGCGGA | |
| | ACTGACTATTCCTTGACTATTTCAAACCTCGAGCAGGAGGACATT | |
| | GCGACATATTTTTGTCAACAAGGTAATACCCTCCCTTACACTTTC | |
| | GGAGGAGGAACCAAACTCGAAATTACCGGGTCCACCAGTGGCTCT | |
| | GGGAAGCCTGGCAGTGGAGAAGGTTCCACTAAAGGCGAGGTGAAG | |
| | CTCCAGGAGAGCGGCCCCGGTCTCGTTGCCCCCAGTCAAAGCCTC | |
| | TCTGTAACGTGCACAGTGAGTGGTGTATCATTGCCTGATTATGGC | |
| | GTCTCCTGGATAAGGCAGCCCCCGCGAAAGGGTCTTGAATGGCTT | |
| | GGGGTAATATGGGGCTCAGAGACAACGTATTATAACTCCGCTCTC | |
| | AAAAGTCGCTTGACGATAATAAAAGATAACTCCAAGAGTCAAGTT | |
| | TTCCTTAAAATGAACAGTTTGCAGACTGACGATACCGCTATATAT | |
| | TATTGTGCTAAACATTATTACTACGGCGGTAGTTACGCGATGGAT | |
| | TATTGGGGGCAGGGGACTTCTGTCACAGTCAGTAGTGCTGCTGCC | |
| | TTTGTCCCGGTATTTCTCCCAGCCAAACCGACCACGACTCCCGCC | |
| | CCGCGCCCTCCGACACCCGCTCCCACCATCGCCTCTCAACCTCTT | |
| | AGTCTTCGCCCCGAGGCATGCCGACCCGCCGCCGGGGGTGCTGTT | |
| | CATACGAGGGGCTTGGACTTCGCTTGTGATATTTACATTTGGGCT | |
| | CCGTTGGCGGGTACGTGCGGCGTCCTTTTGTTGTCACTCGTTATT | |
| | ACTTTGTATTGTAATCACAGGAATCGCTCAAAGCGGAGTAGGTTG | |
| | TTGCATTCCGATTACATGAATATGACTCCTCGCCGGCCTGGGCCG | |
| | ACAAGAAAACATTACCAACCCTATGCCCCCCCACGAGACTTCGCT | |
| | GCGTACAGGTCCCGAGTGAAGTTTTCCCGAAGCGCAGACGCTCCG | |
| | GCATATCAGCAAGGACAGAATCAGCTGTATAACGAACTGAATTTG | |
| | GGACGCCGCGAGGAGTATGACGTGCTTGATAAACGCCGGGGGAGA | |
| | GACCCGGAAATGGGGGGTAAACCCCGAAGAAAGAATCCCCAAGAA | |
| | GGACTCTACAATGAACTCCAGAAGGATAAGATGGCGGAGGCCTAC | |
| | TCAGAAATAGGTATGAAGGGCGAACGACGACGGGGAAAAGGTCAC | |
| | GATGGCCTCTACCAAGGGTTGAGTACGGCAACCAAAGATACGTAC | |
| | GATGCACTGCATATGCAGGCCCTGCCTCCCAGA | |
| Anti-CD19 CAR | MLLLVTSLLLCELPHPAFLLTPDTQMTQTTSSLSASLGDRVTTSC | 73 |
| FMC63-28Z | RASQDTSKYLNWYQQKPDGTVKLLTYHTSRLHSGVPSRFSGSGSG | (with |
| (FMC63-CD8[tm]- | TDYSLTTSNLEQEDTATYFCQQGNTLPYTFGGGTKLETTGSTSGS | signal |
| CD28[co-stimulatory | GKPGSGEGSTKGEVKLQESGPGLVAPSQSLSVTCTVSGVSLPDYG | peptide) |
| domain]-CD3z) | VSWTRQPPRKGLEWLGVTWGSETTYYNSALKSRLTTTKDNSKSQV | 74 |
| Amino Acid | FLKMNSLQTDDTATYYCAKHYYYGGSYAMDYWGQGTSVTVSSAAA | (with no |
| with signal peptide | FVPVFLPAKPTTTPAPRPPTPAPTTASQPLSLRPEACRPAAGGAV | signal |
| | HTRGLDFACDTYTWAPLAGTCGVLLLSLVTTLYCNHRNRSKRSRL | peptide) |
| | LHSDYMNMTPRRPGPTRKHYQPYAPPRDFAAYRSRVKFSRSADAP | |
| | AYQQGQNQLYNELNLGRREEYDVLDKRRGRDPEMGGKPRRKNPQE | |
| | GLYNELQKDKMAEAYSETGMKGERRRGKGHDGLYQGLSTATKDTY | |
| | DALHMQALPPR | |
| Anti-CD19 CAR | DTQMTQTTSSLSASLGDRVTTSCRASQDTSKYLNWYQQKPDGTVK | 75 |
| FMC63-28Z | LLTYHTSRLHSGVPSRFSGSGSGTDYSLTTSNLEQEDTATYFCQQ | |
| (FMC63-CD8[tm]- | GNTLPYTFGGGTKLETTGSTSGSGKPGSGEGSTKGEVKLQESGPG | |
| CD28[co-stimulatory | LVAPSQSLSVTCTVSGVSLPDYGVSWTRQPPRKGLEWLGVTWGSE | |
| domain]-CD3z) | TTYYNSALKSRLTTTKDNSKSQVFLKMNSLQTDDTATYYCAKHYY | |
| Amino Acid | YGGSYAMDYWGQGTSVTVSSAAAFVPVFLPAKPTTTPAPRPPTPA | |
| without signal | PTTASQPLSLRPEACRPAAGGAVHTRGLDFACDTYTWAPLAGTCG | |
| peptide | VLLLSLVTTLYCNHRNRSKRSRLLHSDYMNMTPRRPGPTRKHYQP | |
| | YAPPRDFAAYRSRVKFSRSADAPAYQQGQNQLYNELNLGRREEYD | |
| | VLDKRRGRDPEMGGKPRRKNPQEGLYNELQKDKMAEAYSETGMKG | |
| | ERRRGKGHDGLYQGLSTATKDTYDALHMQALPPR | |
| Anti-CD19 scFv | GATATTCAGATGACTCAGACCACCAGTAGCTTGTCTGCCTCACTG | 76 |
| coding sequence | GGAGACCGAGTAACAATCTCCTGCAGGGCAAGTCAAGACATTAGC | |
| | AAATACCTCAATTGGTACCAGCAGAAGCCCGACGGAACGGTAAAA | |
| | CTCCTCATCTATCATACGTCAAGGTTGCATTCCGGAGTACCGTCA | |
| | CGATTTTCAGGTTCTGGGAGCGGAACTGACTATTCCTTGACTATT | |
| | TCAAACCTCGAGCAGGAGGACATTGCGACATATTTTTGTCAACAA | |
| | GGTAATACCCTCCCTTACACTTTCGGAGGAGGAACCAAACTCGAA | |
| | ATTACCGGGTCCACCAGTGGCTCTGGGAAGCCTGGCAGTGGAGAA | |
| | GGTTCCACTAAAGGCGAGGTGAAGCTCCAGGAGAGCGGCCCCGGT | |
| | CTCGTTGCCCCCAGTCAAAGCCTCTCTGTAACGTGCACAGTGAGT | |
| | GGTGTATCATTGCCTGATTATGGCGTCTCCTGGATAAGGCAGCCC | |
| | CCGCGAAAGGGTCTTGAATGGCTTGGGGTAATATGGGGCTCAGAG | |
| | ACAACGTATTATAACTCCGCTCTCAAAAGTCGCTTGACGATAATA | |
| | AAAGATAACTCCAAGAGTCAAGTTTTCCTTAAAATGAACAGTTTG | |
| | CAGACTGACGATACCGCTATATATTATTGTGCTAAACATTATTAC | |
| | TACGGCGGTAGTTACGCGATGGATTATTGGGGGCAGGGGACTTCT | |
| | GTCACAGTCAGTAGT | |
| Anti-CD19 scFv | DTQMTQTTSSLSASLGDRVTTSCRASQDTSKYLNWYQQKPDGTVK | 77 |
| amino acid sequence | LLTYHTSRLHSGVPSRFSGSGSGTDYSLTTSNLEQEDTATYFCQQ | |
| Linker underlined | GNTLPYTFGGGTKLETTGSTSGSGKPGSGEGSTKGEVKLQESGPG | |
| | LVAPSQSLSVTCTVSGVSLPDYGVSWTRQPPRKGLEWLGVTWGSE | |
| | TTYYNSALKSRLTTTKDNSKSQVFLKMNSLQTDDTATYYCAKHYY | |
| | YGGSYAMDYWGQGTSVTVSS | |
| CD8a extracellular + | GCTGCTGCCTTTGTCCCGGTATTTCTCCCAGCCAAACCGACCACG | 78 |
| CD8a transmembrane + | ACTCCCGCCCCGCGCCCTCCGACACCCGCTCCCACCATCGCCTCT | |
| 5′ Linker | CAACCTCTTAGTCTTCGCCCCGAGGCATGCCGACCCGCCGCCGGG | |
| (underlined) | GGTGCTGTTCATACGAGGGGCTTGGACTTCGCTTGTGATATTTAC | |
| | ATTTGGGCTCCGTTGGCGGGTACGTGCGGCGTCCTTTTGTTGTCA | |
| | CTCGTTATTACTTTGTATTGTAATCACAGGAATCGC | |
| CD8a extracellular + | TTTGTCCCGGTATTTCTCCCAGCCAAACCGACCACGACTCCCGCC | 79 |
| CD8a transmembrane | CCGCGCCCTCCGACACCCGCTCCCACCATCGCCTCTCAACCTCTT | |
| (without linker) | AGTCTTCGCCCCGAGGCATGCCGACCCGCCGCCGGGGGTGCTGTT | |
| | CATACGAGGGGCTTGGACTTCGCTTGTGATATTTACATTTGGGCT | |
| | CCGTTGGCGGGTACGTGCGGCGTCCTTTTGTTGTCACTCGTTATT | |
| | ACTTTGTATTGTAATCACAGGAATCGC | |
| CD8a extracellular + | FVPVFLPAKPTTTPAPRPPTPAPTTASQPLSLRPEACRPAAGGAV | 80 |
| CD8a transmembrane | HTRGLDFACDTYTWAPLAGTCGVLLLSLVTTLYCNHRNR | |
| Anti-CD19 VH | EVKLQESGPGLVAPSQSLSVTCTVSGVSLPDYGVSWTRQPPRKGL | 81 |
| | EWLGVTWGSETTYYNSALKSRLTTTKDNSKSQVFLKMNSLQTDDT | |
| | ATYYCAKHYYYGGSYAMDYWGQGTSVTVSS | |
| Anti-CD19 VL | DTQMTQTTSSLSASLGDRVTTSCRASQDTSKYLNWYQQKPDGTVK | 82 |
| | LLTYHTSRLHSGVPSRFSGSGSGTDYSLTTSNLEQEDTATYFCQQ | |
| | GNTLPYTFGGGTKLETT | |
| CD19 linker | GSTSGSGKPGSGEGSTKG | 83 |

TABLE 6
AAV Donor Template Sequences

| | | SEQ ID |
|---|---|---|
| Name | Sequence | NO: |
| Left ITR (5′ TTR) | TTGGCCACTCCCTCTCTGCGCGCTCGCTCGCTCACTGAGGCCGGG | 84 |
| | CGACCAAAGGTCGCCCGACGCCCGGGCTTTGCCCGGGCGGCCTCA | |
| | GTGAGCGAGCGAGCGCGCAGAGAGGGAGTGGCCAACTCCATCACT | |
| | AGGGGTTCCT | |
| Left ITR (5′ TTR) | CCTGCAGGCAGCTGCGCGCTCGCTCGCTCACTGAGGCCGCCCGGG | 85 |
| (alternate) | CGTCGGGCGACCTTTGGTCGCCCGGCCTCAGTGAGCGAGCGAGCG | |
| | CGCAGAGAGGGAGTGGCCAACTCCATCACTAGGGGTTCCT | |
| Right ITR (3′ TTR) | AGGAACCCCTAGTGATGGAGTTGGCCACTCCCTCTCTGCGCGCTC | 86 |
| | GCTCGCTCACTGAGGCCGCCCGGGCAAAGCCCGGGCGTCGGGCGA | |
| | CCTTTGGTCGCCCGGCCTCAGTGAGCGAGCGAGCGCGCAGAGAGG | |
| | GAGTGGCCAA | |
| Right ITR (3′ TTR) | AGGAACCCCTAGTGATGGAGTTGGCCACTCCCTCTCTGCGCGCTC | 87 |
| (alternate) | GCTCGCTCACTGAGGCCGGGCGACCAAAGGTCGCCCGACGCCCGG | |
| | GCTTTGCCCGGGCGGCCTCAGTGAGCGAGCGAGCGCGCAGCTGCC | |
| | TGCAGG | |
| TRAC-LHA (800 bp) | GAGATGTAAGGAGCTGCTGTGACTTGCTCAAGGCCTTATATCGAG | 88 |
| | TAAACGGTAGTGCTGGGGCTTAGACGCAGGTGTTCTGATTTATAG | |
| | TTCAAAACCTCTATCAATGAGAGAGCAATCTCCTGGTAATGTGAT | |
| | AGATTTCCCAACTTAATGCCAACATACCATAAACCTCCCATTCTG | |
| | CTAATGCCCAGCCTAAGTTGGGGAGACCACTCCAGATTCCAAGAT | |
| | GTACAGTTTGCTTTGCTGGGCCTTTTTCCCATGCCTGCCTTTACT | |
| | CTGCCAGAGTTATATTGCTGGGGTTTTGAAGAAGATCCTATTAAA | |
| | TAAAAGAATAAGCAGTATTATTAAGTAGCCCTGCATTTCAGGTTT | |
| | CCTTGAGTGGCAGGCCAGGCCTGGCCGTGAACGTTCACTGAAATC | |
| | ATGGCCTCTTGGCCAAGATTGATAGCTTGTGCCTGTCCCTGAGTC | |
| | CCAGTCCATCACGAGCAGCTGGTTTCTAAGATGCTATTTCCCGTA | |
| | TAAAGCATGAGACCGTGACTTGCCAGCCCCACAGAGCCCCGCCCT | |
| | TGTCCATCACTGGCATCTGGACTCCAGCCTGGGTTGGGGCAAAGA | |
| | GGGAAATGAGATCATGTCCTAACCCTGATCCTCTTGTCCCACAGA | |
| | TATCCAGAACCCTGACCCTGCCGTGTACCAGCTGAGAGACTCTAA | |
| | ATCCAGTGACAAGTCTGTCTGCCTATTCACCGATTTTGATTCTCA | |
| | AACAAATGTGTCACAAAGTAAGGATTCTGATGTGTATATCACAGA | |
| | CAAAACTGTGCTAGACATGAGGTCTATGGACTTCA | |
| TRAC-RHA (800 bp) | TGGAGCAACAAATCTGACTTTGCATGTGCAAACGCCTTCAACAAC | 89 |
| | AGCATTATTCCAGAAGACACCTTCTTCCCCAGCCCAGGTAAGGGC | |
| | AGCTTTGGTGCCTTCGCAGGCTGTTTCCTTGCTTCAGGAATGGCC | |
| | AGGTTCTGCCCAGAGCTCTGGTCAATGATGTCTAAAACTCCTCTG | |
| | ATTGGTGGTCTCGGCCTTATCCATTGCCACCAAAACCCTCTTTTT | |
| | ACTAAGAAACAGTGAGCCTTGTTCTGGCAGTCCAGAGAATGACAC | |
| | GGGAAAAAAGCAGATGAAGAGAAGGTGGCAGGAGAGGGCACGTGG | |
| | CCCAGCCTCAGTCTCTCCAACTGAGTTCCTGCCTGCCTGCCTTTG | |
| | CTCAGACTGTTTGCCCCTTACTGCTCTTCTAGGCCTCATTCTAAG | |
| | CCCCTTCTCCAAGTTGCCTCTCCTTATTTCTCCCTGTCTGCCAAA | |
| | AAATCTTTCCCAGCTCACTAAGTCAGTCTCACGCAGTCACTCATT | |
| | AACCCACCAATCACTGATTGTGCCGGCACATGAATGCACCAGGTG | |
| | TTGAAGTGGAGGAATTAAAAAGTCAGATGAGGGGTGTGCCCAGAG | |
| | GAAGCACCATTCTAGTTGGGGGAGCCCATCTGTCAGCTGGGAAAA | |
| | GTCCAAATAACTTCAGATTGGAATGTGTTTTAACTCAGGGTTGAG | |
| | AAAACAGCTACCTTCAGGACAAAAGTCAGGGAAGGGCTCTCTGAA | |
| | GAAATGCTACTTGAAGATACCAGCCCTACCAAGGGCAGGGAGAGG | |
| | ACCCTATAGAGGCCTGGGACAGGAGCTCAATGAGAAAGG | |
| EF1a | GGCTCCGGTGCCCGTCAGTGGGCAGAGCGCACATCGCCCACAGTC | 90 |
| | CCCGAGAAGTTGGGGGGAGGGGTCGGCAATTGAACCGGTGCCTAG | |
| | AGAAGGTGGCGCGGGGTAAACTGGGAAAGTGATGTCGTGTACTGG | |
| | CTCCGCCTTTTTCCCGAGGGTGGGGGAGAACCGTATATAAGTGCA | |
| | GTAGTCGCCGTGAACGTTCTTTTTCGCAACGGGTTTGCCGCCAGA | |
| | ACACAGGTAAGTGCCGTGTGTGGTTCCCGCGGGCCTGGCCTCTTT | |
| | ACGGGTTATGGCCCTTGCGTGCCTTGAATTACTTCCACTGGCTGC | |
| | AGTACGTGATTCTTGATCCCGAGCTTCGGGTTGGAAGTGGGTGGG | |
| | AGAGTTCGAGGCCTTGCGCTTAAGGAGCCCCTTCGCCTCGTGCTT | |
| | GAGTTGAGGCCTGGCCTGGGCGCTGGGGCCGCCGCGTGCGAATCT | |
| | GGTGGCACCTTCGCGCCTGTCTCGCTGCTTTCGATAAGTCTCTAG | |
| | CCATTTAAAATTTTTGATGACCTGCTGCGACGCTTTTTTTCTGGC | |
| | AAGATAGTCTTGTAAATGCGGGCCAAGATCTGCACACTGGTATTT | |
| | CGGTTTTTGGGGCCGCGGGCGGCGACGGGGCCCGTGCGTCCCAGC | |
| | GCACATGTTCGGCGAGGCGGGGCCTGCGAGCGCGGCCACCGAGAA | |
| | TCGGACGGGGGTAGTCTCAAGCTGGCCGGCCTGCTCTGGTGCCTG | |
| | GCCTCGCGCCGCCGTGTATCGCCCCGCCCTGGGCGGCAAGGCTGG | |
| | CCCGGTCGGCACCAGTTGCGTGAGCGGAAAGATGGCCGCTTCCCG | |
| | GCCCTGCTGCAGGGAGCTCAAAATGGAGGACGCGGCGCTCGGGAG | |
| | AGCGGGCGGGTGAGTCACCCACACAAAGGAAAAGGGCCTTTCCGT | |
| | CCTCAGCCGTCGCTTCATGTGACTCCACGGAGTACCGGGCGCCGT | |
| | CCAGGCACCTCGATTAGTTCTCGAGCTTTTGGAGTACGTCGTCTT | |
| | TAGGTTGGGGGGAGGGGTTTTATGCGATGGAGTTTCCCCACACTG | |
| | AGTGGGTGGAGACTGAAGTTAGGCCAGCTTGGCACTTGATGTAAT | |
| | TCTCCTTGGAATTTGCCCTTTTTGAGTTTGGATCTTGGTTCATTC | |
| | TCAAGCCTCAGACAGTGGTTCAAAGTTTTTTTCTTCCATTTCAGG | |
| | TGTCGTGA | |
| CD19 | GAGATGTAAGGAGCTGCTGTGACTTGCTCAAGGCCTTATATCGAG | 91 |
| LHA to RHA | TAAACGGTAGTGCTGGGGCTTAGACGCAGGTGTTCTGATTTATAG | |
| | TTCAAAACCTCTATCAATGAGAGAGCAATCTCCTGGTAATGTGAT | |
| | AGATTTCCCAACTTAATGCCAACATACCATAAACCTCCCATTCTG | |
| | CTAATGCCCAGCCTAAGTTGGGGAGACCACTCCAGATTCCAAGAT | |
| | GTACAGTTTGCTTTGCTGGGCCTTTTTCCCATGCCTGCCTTTACT | |
| | CTGCCAGAGTTATATTGCTGGGGTTTTGAAGAAGATCCTATTAAA | |
| | TAAAAGAATAAGCAGTATTATTAAGTAGCCCTGCATTTCAGGTTT | |
| | CCTTGAGTGGCAGGCCAGGCCTGGCCGTGAACGTTCACTGAAATC | |
| | ATGGCCTCTTGGCCAAGATTGATAGCTTGTGCCTGTCCCTGAGTC | |
| | CCAGTCCATCACGAGCAGCTGGTTTCTAAGATGCTATTTCCCGTA | |
| | TAAAGCATGAGACCGTGACTTGCCAGCCCCACAGAGCCCCGCCCT | |
| | TGTCCATCACTGGCATCTGGACTCCAGCCTGGGTTGGGGCAAAGA | |
| | GGGAAATGAGATCATGTCCTAACCCTGATCCTCTTGTCCCACAGA | |
| | TATCCAGAACCCTGACCCTGCCGTGTACCAGCTGAGAGACTCTAA | |
| | ATCCAGTGACAAGTCTGTCTGCCTATTCACCGATTTTGATTCTCA | |
| | AACAAATGTGTCACAAAGTAAGGATTCTGATGTGTATATCACAGA | |
| | CAAAACTGTGCTAGACATGAGGTCTATGGACTTCAGGCTCCGGTG | |
| | CCCGTCAGTGGGCAGAGCGCACATCGCCCACAGTCCCCGAGAAGT | |
| | TGGGGGGAGGGGTCGGCAATTGAACCGGTGCCTAGAGAAGGTGGC | |
| | GCGGGGTAAACTGGGAAAGTGATGTCGTGTACTGGCTCCGCCTTT | |
| | TTCCCGAGGGTGGGGGAGAACCGTATATAAGTGCAGTAGTCGCCG | |
| | TGAACGTTCTTTTTCGCAACGGGTTTGCCGCCAGAACACAGGTAA | |
| | GTGCCGTGTGTGGTTCCCGCGGGCCTGGCCTCTTTACGGGTTATG | |
| | GCCCTTGCGTGCCTTGAATTACTTCCACTGGCTGCAGTACGTGAT | |
| | TCTTGATCCCGAGCTTCGGGTTGGAAGTGGGTGGGAGAGTTCGAG | |
| | GCCTTGCGCTTAAGGAGCCCCTTCGCCTCGTGCTTGAGTTGAGGC | |
| | CTGGCCTGGGCGCTGGGGCCGCCGCGTGCGAATCTGGTGGCACCT | |
| | TCGCGCCTGTCTCGCTGCTTTCGATAAGTCTCTAGCCATTTAAAA | |
| | TTTTTGATGACCTGCTGCGACGCTTTTTTTCTGGCAAGATAGTCT | |
| | TGTAAATGCGGGCCAAGATCTGCACACTGGTATTTCGGTTTTTGG | |
| | GGCCGCGGGCGGCGACGGGGCCCGTGCGTCCCAGCGCACATGTTC | |
| | GGCGAGGCGGGGCCTGCGAGCGCGGCCACCGAGAATCGGACGGGG | |
| | GTAGTCTCAAGCTGGCCGGCCTGCTCTGGTGCCTGGCCTCGCGCC | |
| | GCCGTGTATCGCCCCGCCCTGGGCGGCAAGGCTGGCCCGGTCGGC | |
| | ACCAGTTGCGTGAGCGGAAAGATGGCCGCTTCCCGGCCCTGCTGC | |
| | AGGGAGCTCAAAATGGAGGACGCGGCGCTCGGGAGAGCGGGCGGG | |
| | TGAGTCACCCACACAAAGGAAAAGGGCCTTTCCGTCCTCAGCCGT | |
| | CGCTTCATGTGACTCCACGGAGTACCGGGCGCCGTCCAGGCACCT | |
| | CGATTAGTTCTCGAGCTTTTGGAGTACGTCGTCTTTAGGTTGGGG | |
| | GGAGGGGTTTTATGCGATGGAGTTTCCCCACACTGAGTGGGTGGA | |
| | GACTGAAGTTAGGCCAGCTTGGCACTTGATGTAATTCTCCTTGGA | |
| | ATTTGCCCTTTTTGAGTTTGGATCTTGGTTCATTCTCAAGCCTCA | |
| | GACAGTGGTTCAAAGTTTTTTTCTTCCATTTCAGGTGTCGTGACC | |
| | ACCATGCTTCTTTTGGTTACGTCTCTGTTGCTTTGCGAACTTCCT | |
| | CATCCAGCGTTCTTGCTGATCCCCGATATTCAGATGACTCAGACC | |
| | ACCAGTAGCTTGTCTGCCTCACTGGGAGACCGAGTAACAATCTCC | |
| | TGCAGGGCAAGTCAAGACATTAGCAAATACCTCAATTGGTACCAG | |
| | CAGAAGCCCGACGGAACGGTAAAACTCCTCATCTATCATACGTCA | |
| | AGGTTGCATTCCGGAGTACCGTCACGATTTTCAGGTTCTGGGAGC | |
| | GGAACTGACTATTCCTTGACTATTTCAAACCTCGAGCAGGAGGAC | |
| | ATTGCGACATATTTTTGTCAACAAGGTAATACCCTCCCTTACACT | |
| | TTCGGAGGAGGAACCAAACTCGAAATTACCGGGTCCACCAGTGGC | |
| | TCTGGGAAGCCTGGCAGTGGAGAAGGTTCCACTAAAGGCGAGGTG | |
| | AAGCTCCAGGAGAGCGGCCCCGGTCTCGTTGCCCCCAGTCAAAGC | |
| | CTCTCTGTAACGTGCACAGTGAGTGGTGTATCATTGCCTGATTAT | |
| | GGCGTCTCCTGGATAAGGCAGCCCCCGCGAAAGGGTCTTGAATGG | |
| | CTTGGGGTAATATGGGGCTCAGAGACAACGTATTATAACTCCGCT | |
| | CTCAAAAGTCGCTTGACGATAATAAAAGATAACTCCAAGAGTCAA | |
| | GTTTTCCTTAAAATGAACAGTTTGCAGACTGACGATACCGCTATA | |
| | TATTATTGTGCTAAACATTATTACTACGGCGGTAGTTACGCGATG | |
| | GATTATTGGGGGCAGGGGACTTCTGTCACAGTCAGTAGTGCTGCT | |
| | GCCTTTGTCCCGGTATTTCTCCCAGCCAAACCGACCACGACTCCC | |
| | GCCCCGCGCCCTCCGACACCCGCTCCCACCATCGCCTCTCAACCT | |
| | CTTAGTCTTCGCCCCGAGGCATGCCGACCCGCCGCCGGGGGTGCT | |
| | GTTCATACGAGGGGCTTGGACTTCGCTTGTGATATTTACATTTGG | |
| | GCTCCGTTGGCGGGTACGTGCGGCGTCCTTTTGTTGTCACTCGTT | |
| | ATTACTTTGTATTGTAATCACAGGAATCGCTCAAAGCGGAGTAGG | |
| | TTGTTGCATTCCGATTACATGAATATGACTCCTCGCCGGCCTGGG | |
| | CCGACAAGAAAACATTACCAACCCTATGCCCCCCCACGAGACTTC | |
| | GCTGCGTACAGGTCCCGAGTGAAGTTTTCCCGAAGCGCAGACGCT | |
| | CCGGCATATCAGCAAGGACAGAATCAGCTGTATAACGAACTGAAT | |
| | TTGGGACGCCGCGAGGAGTATGACGTGCTTGATAAACGCCGGGGG | |
| | AGAGACCCGGAAATGGGGGGTAAACCCCGAAGAAAGAATCCCCAA | |
| | GAAGGACTCTACAATGAACTCCAGAAGGATAAGATGGCGGAGGCC | |
| | TACTCAGAAATAGGTATGAAGGGCGAACGACGACGGGGAAAAGGT | |
| | CACGATGGCCTCTACCAAGGGTTGAGTACGGCAACCAAAGATACG | |
| | TACGATGCACTGCATATGCAGGCCCTGCCTCCCAGATAATAATAA | |
| | AATCGCTATCCATCGAAGATGGATGTGTGTTGGTTTTTTGTGTGT | |
| | GGAGCAACAAATCTGACTTTGCATGTGCAAACGCCTTCAACAACA | |
| | GCATTATTCCAGAAGACACCTTCTTCCCCAGCCCAGGTAAGGGCA | |
| | GCTTTGGTGCCTTCGCAGGCTGTTTCCTTGCTTCAGGAATGGCCA | |
| | GGTTCTGCCCAGAGCTCTGGTCAATGATGTCTAAAACTCCTCTGA | |
| | TTGGTGGTCTCGGCCTTATCCATTGCCACCAAAACCCTCTTTTTA | |
| | CTAAGAAACAGTGAGCCTTGTTCTGGCAGTCCAGAGAATGACACG | |
| | GGAAAAAAGCAGATGAAGAGAAGGTGGCAGGAGAGGGCACGTGGC | |
| | CCAGCCTCAGTCTCTCCAACTGAGTTCCTGCCTGCCTGCCTTTGC | |
| | TCAGACTGTTTGCCCCTTACTGCTCTTCTAGGCCTCATTCTAAGC | |
| | CCCTTCTCCAAGTTGCCTCTCCTTATTTCTCCCTGTCTGCCAAAA | |
| | AATCTTTCCCAGCTCACTAAGTCAGTCTCACGCAGTCACTCATTA | |
| | ACCCACCAATCACTGATTGTGCCGGCACATGAATGCACCAGGTGT | |
| | TGAAGTGGAGGAATTAAAAAGTCAGATGAGGGGTGTGCCCAGAGG | |
| | AAGCACCATTCTAGTTGGGGGAGCCCATCTGTCAGCTGGGAAAAG | |
| | TCCAAATAACTTCAGATTGGAATGTGTTTTAACTCAGGGTTGAGA | |
| | AAACAGCTACCTTCAGGACAAAAGTCAGGGAAGGGCTCTCTGAAG | |
| | AAATGCTACTTGAAGATACCAGCCCTACCAAGGGCAGGGAGAGGA | |
| | CCCTATAGAGGCCTGGGACAGGAGCTCAATGAGAAAGG | |

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (1RL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1. Anti-CD19 CAR-T Cell Production and Characterization

Allogeneic human T cells that lack expression of the TRAC gene, β2M gene, Regnase-1 gene, and TGFBRII gene, and express a chimeric antigen receptor (CAR) targeting CD19 (e.g., CTX112 cells) were produced. The following sgRNAs were used for the production: TA-1 (SEQ ID NO: 2) (targeting TRAC to eliminate surface expression of TCR α and β chains), β2M-1 (SEQ ID NO: 6) (targeting β2M to eliminate MHC class I surface expression), TGFBRII-5 (SEQ ID NO: 14) (targeting TGFBRII, an initiator of TGF-β signaling), and ZC3H12A-10 (SEQ ID NO: 10) (targeting Regnase-1, an immune-reaction negative regulator). CRISPR-Cas9 editing at the TRAC locus facilitated the integration of rAAV-138, which encodes the anti-CD19 CAR of SEQ ID NO:73. The anti-CD19 CAR T cells with TRAC gene, β2M gene, Regnase-1 gene and TGFBRII gene knockouts are denoted as CD19 CAR+TRAC KO+β2M KO+R/T KO.

Non-Good Manufacturing Practice (GMP) lots of CD19 CAR+TRAC KO+β2M KO+R/T KO were produced at both small scale and at manufacturing scale from 3 healthy donors each. Small-scale lots (Table 7) showed 55.6±3% CAR cell surface expression, 94.3±3.3% loss of surface TCRαβ (without TCRαβ depletion), 72.8±8.7% loss of B2M surface expression, and indel frequencies of 84.4±4.3 and 96.4±1% for TGFBRII and Regnase-1, respectively. Manufacturing-scale lots (Table 8) showed 63.3±6.2% cell surface CAR expression, 99.7±0.3% loss of surface TCRαβ (with TCRαβ depletion), 82.8±0.2% loss of β2M surface expression, and indel frequencies of 84.9±3.3 and 95.3±1.5% for TGFBR2 and Regnase-1, respectively.

TABLE 7
Small-Scale Non-GMP Lots

| T Cell Lot | Donor 1 | Donor 2 | Donor 3 | Average | S.D. |
|---|---|---|---|---|---|
| % TCRab⁻ | 91.3 | 97.8 | 93.8 | 94.3 | 3.3 |
| % β2M⁻ | 63.8 | 81.1 | 73.5 | 72.8 | 8.7 |
| % Regnase-1 indel | 97.0 | 97.0 | 95.3 | 96.4 | 1.0 |
| % TGFBR2 indel | 83.6 | 89.0 | 80.6 | 84.4 | 4.3 |
| % CAR⁺ | 57.8 | 52.1 | 56.9 | 55.6 | 3.1 |

β2M: β-2 microglobulin; CAR: chimeric antigen receptor; GMP: Good Manufacturing Practice; indel: insertion/deletion; Regnase: regulatory RNase; S.D.: standard deviation; TCRαβ: T cell receptor alpha and beta chains; TGFBR2: transforming growth factor β receptor 2.

T cells from 3 individual donors were thawed and activated using TransAct beads for 48 hours in fully supplemented media (containing 5% human AB serum, IL-2, and IL-7.) On Day 2, the T cells were electroporated with RNPs containing Cas9 and gRNA targeting the Regnase-1 and TGFBRII loci. The edited cells were then seeded in fully supplemented media and cultured for 48 hours. On Day 4, the cells were electroporated with RNPs containing Cas9 and gRNAs targeting the TRAC and β2M loci. This was followed by incubation using AAV6 containing an HDR template encoding CD19 CAR. On Day 8, the cells were supplemented with IL-2 and IL-7. When cells reached a density of 3×10⁶ cells/mL (±10%), they were harvested and cryopreserved in CS5 buffer at 50×10⁶ cells/mL. Staining was performed using antibodies against TRAC and β2M proteins, whereas CAR expression was detected through staining with anti-idiotypic antibody labeled with biotin, followed by incubation with fluorescent streptavidin. The editing efficiencies of the TGFBR2 and Regnase-1 gRNAs and Cas9 were assessed by thawing the cryopreserved DP and extracting genomic DNA. This was followed by Sanger sequencing and indel analysis using tracking of indels by decomposition (TIDE) analysis.

TABLE 8
Manufacturing-Scale Non-GMP Lots

| T Cell Lot | Donor 1 | Donor 2 | Donor 3 | Average | S.D. |
|---|---|---|---|---|---|
| % TCRαβ⁻ | 99.9 | 99.9 | 99.4 | 99.7 | 0.3 |
| % B2M⁻ | 82.6 | 83.0 | 82.7 | 82.8 | 0.2 |
| % Regnase-1 indel | 97.1 | 94.3 | 94.6 | 95.3 | 1.5 |
| % TGFBR2 indel | 85.8 | 87.7 | 81.3 | 84.9 | 3.3 |
| % CAR⁺ | 56.2 | 67.6 | 66.1 | 63.3 | 6.2 |

Cells from 3 donors were produced and analyzed as described above but at manufacturing-level scale. These lots were also subjected to TCRαβ depletion when cell reached a density of 3×10⁶ cells/mL (±10%). Briefly, TCR⁺ cells were depleted using a magnetic column by incubating with anti-TCRαβ-biotin beads followed by incubation with anti-biotin beads. The TCR-depleted cells were seeded into fully supplemented media, and 16 to 20 hours after TCR depletion, the cells were harvested and cryopreserved in CS5 buffer at 50×10⁶ cells/mL.

### Example 2. TGF-β Effects on In Vitro Cell Expansion

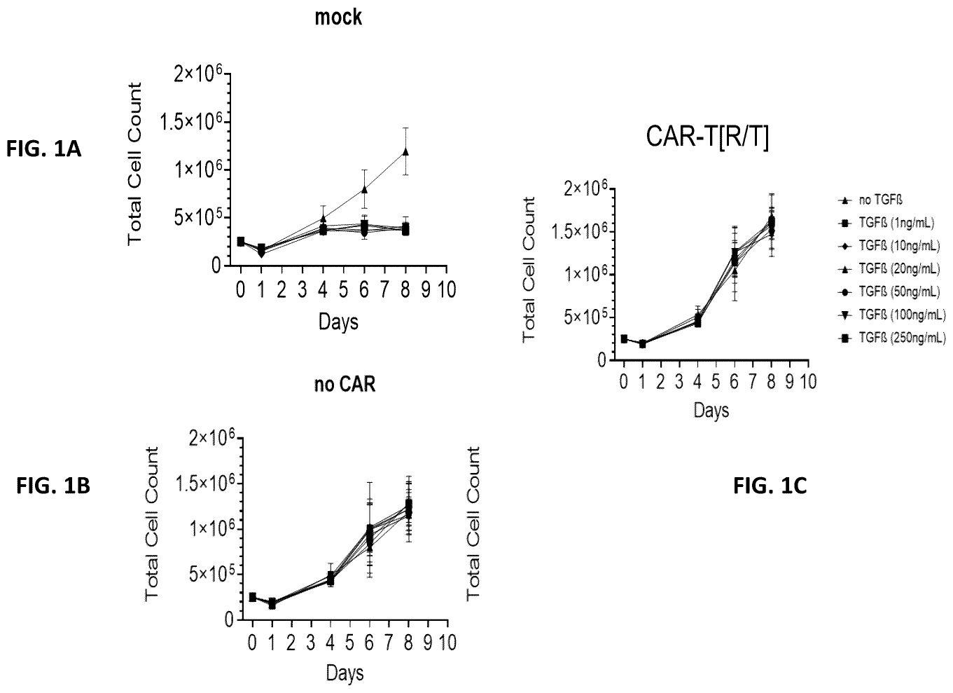

The ability of TGF-β to inhibit the growth of engineered CAR T cells with TRAC, β2M, Reg1 and TGFBRII (R/T) knockouts was assessed. The results show that TGF-β was able to inhibit the growth of mock-electroporated human T cells from 3 donors. TGF-β was unable to inhibit the growth of T cells edited to lack TGFBRII (along with disruptions in the TRAC, β2M, and Regnase-1 loci) either expressing an anti-CD19 CAR or not (no CAR) (see, FIGS. 1A-1C). The data suggest that TGFBRII disruption eliminates the inhibitory effects of TGF-β on human T cell in vitro expansion.

### Example 3. Effector Cytokine Release by Anti-CD19 CAR T Cells

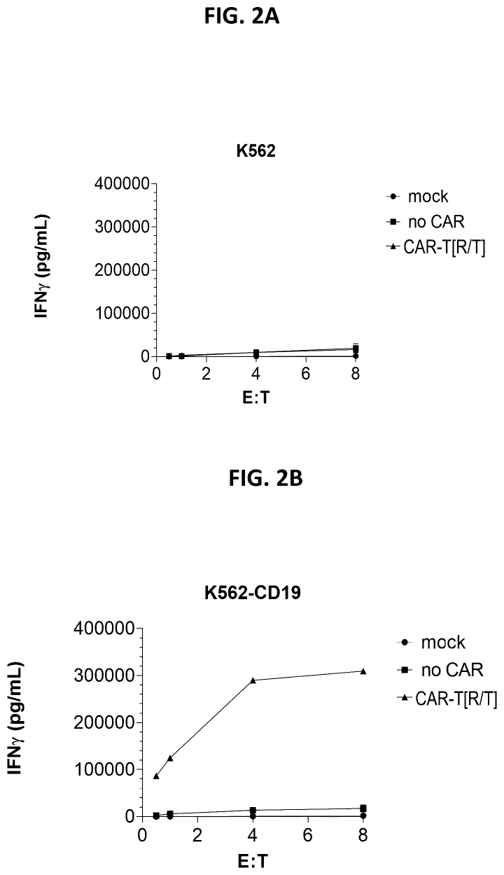

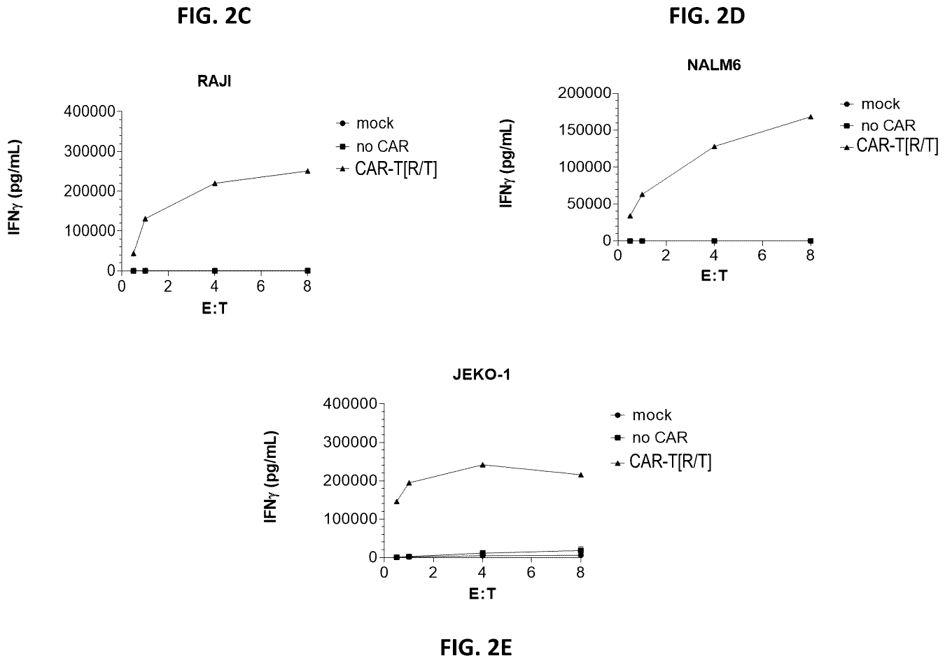

Three lots of the CD19 CAR+TRAC KO+β2M KO+R/T KO T cells were produced from 3 unique donors. Such T cells did not secrete high levels of IFN-γ in the presence of the CD19-negative K562 cell line, but secreted high levels of IFN-7 when CD19 was expressed in K562 cells (K562-CD19) as well as in the presence of 3 different human CD19-positive leukemia/lymphoma cell lines (FIGS. 2A-2E). Cells that contained the TRAC, β2M, Reg1, and TGFBRII edits except for the inserted CAR (no CAR) did not express significant levels of IFN-7. The data suggest that the CD19 CAR+TRAC KO+β2M KO+R/T KO T cells secrete IFN-7 selectively in the presence of CD19 and additional edits do not change this specificity.

### Example 4. Cytotoxicity of Anti-CD19 CAR T Cells Against Tumor Cells

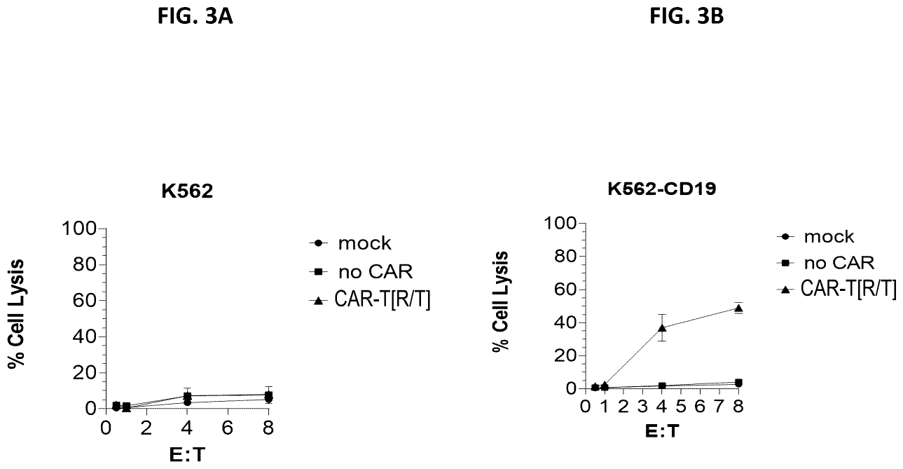

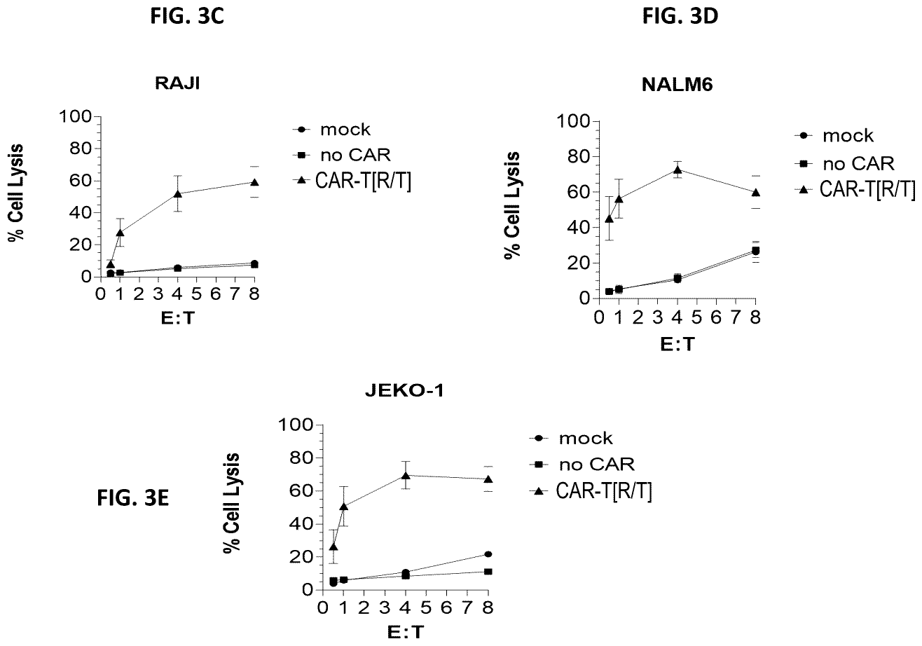

The ability of the CD19 CAR+TRAC KO+β2M KO+R/T KO T cells to kill CD19-expressing cells was assessed. Three lots of the CAR T cells were produced from 3 unique donors. Such T cells did not show levels of cytotoxic activity against the CD19-negative cell line K562 above control cells that were either TCR⁺ T cells that were mock electroporated (mock) or cells containing the TRAC, β2M, Reg1, and TGFBRII edits except for CAR insertion (no CAR). CD19 CAR+TRAC KO+β2M KO+R/T KO T cells displayed high levels of cytotoxicity in K562 cells engineered to express human CD19 (K562-CD19) as well as against 3 different CD19-expressing human leukemia/lymphoma cell lines (FIGS. 3A-3E). The data suggest that CD19 CAR+TRAC KO+β2M KO+R/T KO T cells selectively kill CD19-positive cells and additional edits do not change this specificity.

### Example 5. RNA Sequencing of Activated CAR T Cells

To assess the effects of Regnase-1 and TGFBRII on gene expression, RNA sequencing was performed on various engineered CAR T cells, including single additional edit controls (CD19 CAR disrupted for TRAC and β2M; CD19 CAR disrupted for TRAC, β2M and TGFBRII; and CD19 CAR disrupted for TRAC, β2M, Regnase-1), and CD19 CAR+TRAC KO+β2M KO+R/T KO (CD19 CAR disrupted for TRAC, β2M, TGFBRII, and Regnase-1) in the presence and absence of CD19-positive target cells (Nalm6) at 0 (TO) and 4 hours (T4) after T cell activation. A group of genes that were differentially expressed across both time points (30 upregulated genes and 13 downregulated genes) between anti-CD19 CAR-T cells having disrupted TGFBRII and Regnase-1 genes (CTX112 cells) and the counterpart CAR-T cells having no disruptions in TGFBRII and Regnase-1 genes (were evaluated for their putative functions based on published literature. Both TGFBR2 and Regnase-1 have been implicated in numerous pathways governing immune regulation and inflammatory responses. See, e.g., Batlle and Massague, 2019, Immunity 50, 924-940; Flavell et al., 2010, Nat Rev Immunol 10, 554-567; Kidoya et al., 2019, Nature Communications 10, 1072; Sanjabi et al., 2017, Cold Spring Harb Perspect Biol 9; Takeuchi, 2018, Wiley Interdiscip Rev RNA 9; and Uehata et al., 2013, Cell 153, 1036-1049).

Differential expression of several genes involved in regulating T cell function was observed in CTX112 cells. These include downregulation of forkhead box P3 (FOXP3), a transcription factor reported to be essential for suppression of activated T cells and antitumor immunity by regulatory T cells (Schmidt et al., 2012), of zinc finger BED-type containing 2 (ZBED2), a transcription factor implicated in T cell dysfunction (Li et al., 2019), and of transcription factor 7 (TCF7), another transcription factor involved in T cell development and differentiation (Zhang et al., 2021). Additionally, CTX112 cells showed upregulation of neural cell adhesion molecule 1 (NCAM1), which has been reported to promote CAR T cell survival and antitumor response (Zou et al., 2019).

Several genes that are implicated in inflammatory responses show differential expression in CTX112, at both TO and T4. Examples include pro- and anti-inflammation genes such as IL17F, CXCL8, IL19, and IRAK3, which show differences in RNA expression in CTX112, as compared with the counterpart cells.

Finally, the differentially expressed genes were annotated against a list of cancer-associated genes generated by combining 3 expert-curated data resources (Chakravarty et al., 2017; Tate et al., 2019; Walker et al., 2012). Aside from downregulation of TGFBR2, which reflects the intended effect of CRISPR-Cas9 editing, none of the 43 differentially expressed genes in CTX112 cells was among the broad list of 1,162 cancer-associated genes, consistent with a lack of oncogenicity as supported by other nonclinical studies.

Overall, the observed differences in RNA expression between CTX112 cells and the counterpart CAR-T cells are consistent with gene signatures affecting T cell and immune function. These findings may correlate with enhanced proliferative capacity and antitumor activity observed with CTX112 cells.

### Example 6. Assessment of TGFBRII and/or Regnase-1 Knockouts

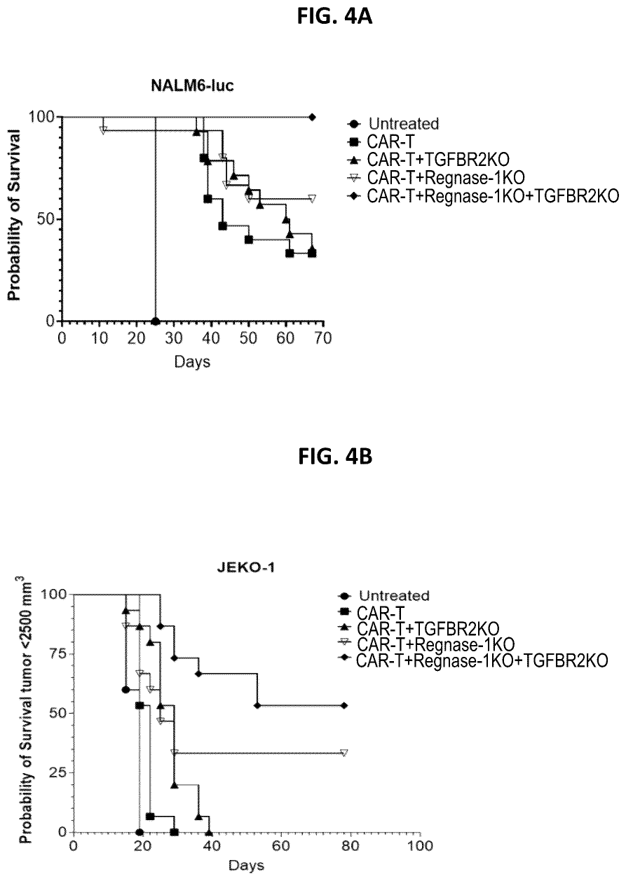

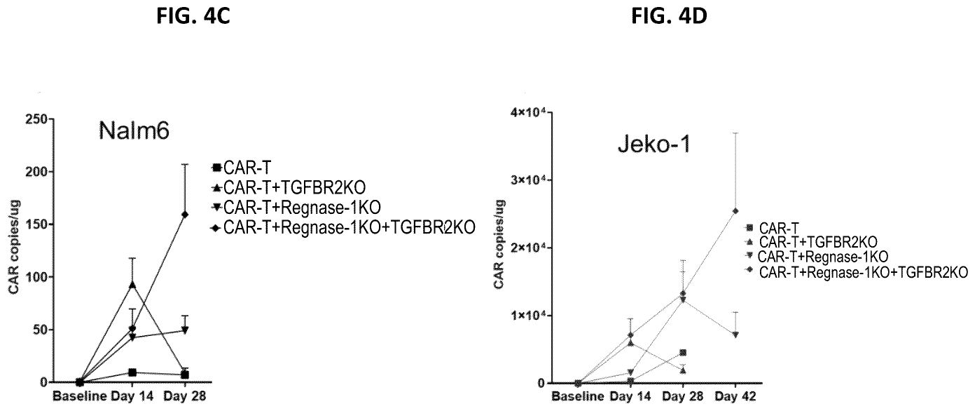

NOG mice were inoculated with either (A) Nalm6 leukemia cells intravenously or (B) Jeko-1 cells subcutaneously. Mice were infused with the indicated CAR T cells at 4×10⁶ CAR⁺ cells/mouse, and survival of mice is shown in FIGS. 4A-4B. CAR T cells were produced from 3 unique donors for both models; in each model, 5 mice were used as controls and 5 mice in each study received cells produced from each donor (15 total). ddPCR (droplet digital polymerase chain reaction) was performed on DNA isolated from peripheral blood cells of mice from the indicated CAR T cell group to detect integrated CAR⁺ human cells. The number of CAR copies per mg of DNA is indicated ±S.E.M (standard error of the mean) (FIGS. 4C-4D).

The results show that further genetic disruption of both TGFBRII and Regnase-1 in CD19 CAR+TRAC KO+β2M KO T cells synergistically increased survival of both the CD19⁺ Nalm6 leukemia and the Jeko-1 lymphoma models in immunocompromised mice (P<0.0001 log-rank [Mantel-Cox] test) for both models). Although either TGFBRII or Regnase-1 could individually increase survival in the Jeko-1 model (P<0.0006 and P=0.019, respectively), neither edit alone could increase survival relative to CD19 CAR+TRAC KO+β2M KO in both models, nor to a greater magnitude than TGFBRII/Regnase-1 double-deficient cells. This increased efficacy was correlated with synergistically increased expansion and persistence of the CD19 CAR+TRAC KO+β2M KO+R/T KO T cells relative to CD19 CAR+TRAC KO+β2M KO T cells or CD19 CAR+TRAC KO+β2M KO T cells with either just TGFBRII knockout or Regnase-1 knockout in both Nalm6 and Jeko-1 models. These data suggest that TGFBRII and Regnase-1 disruptions synergistically act to increase expansion and the functional persistence of CAR T cells. That is, TGFBRII and Regnase-1 disruption synergistically increased potency of the CD19 CAR+TRAC KO+β2M KO T cells against CD19-positive malignancies.

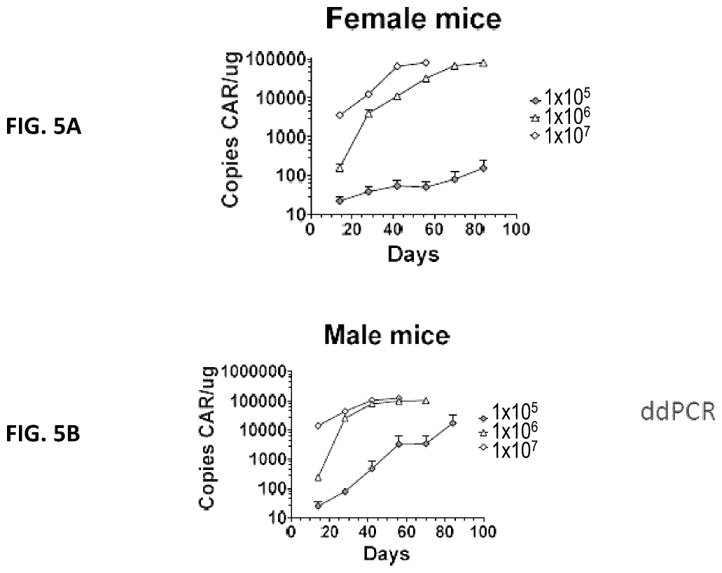

Further assessment shows that polyclonal anti-CD19 CAR T cells with TRAC, β2M, and R/T knockouts were persistent in both female and male mice (see, FIGS. 5A-5B).

### Example 7. Therapeutic Efficacy of Anti-CD19 CAR-T Cells in CD19⁺ Leukemia and Lymphoma Xenograft Models

The CD19 CAR+TRAC KO+β2M KO+R/T KO T cells were infused into NSG mice bearing either Raji-luciferase lymphoma cells (disseminated) or Nalm6-leukemia cells (disseminated) 3 days post-inoculation of the tumor cells. For the Jeko-1 experiment, the T cells were infused into NSG mice bearing Jeko-1 lymphoma cells (subcutaneous) when tumors reached 150 mm³.

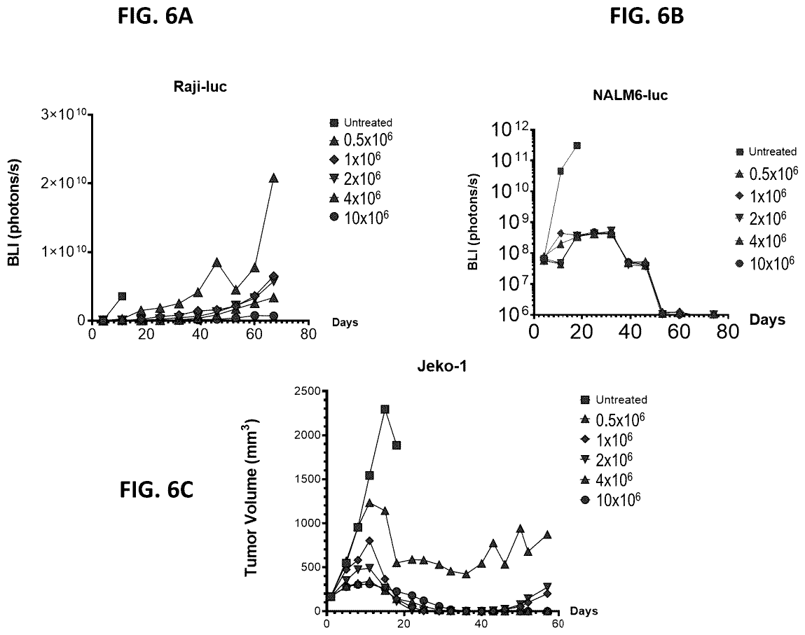

The results showed high levels of tumor control in all 3 models (FIGS. 6A-6C). Levels of leukemia or lymphoma are shown in surviving mice over time after tumor cell inoculation (Day 0). Notably, the CD19 CAR+TRAC KO+β2M KO+R/T KO T cells displayed high levels of control of the Jeko-1 lymphoma model at doses lower than a sub-efficacious dose of CD19 CAR+TRAC KO+β2M KO (<4×10⁶ CAR⁺ cells/mouse) (see, FIGS. 4A-4D).

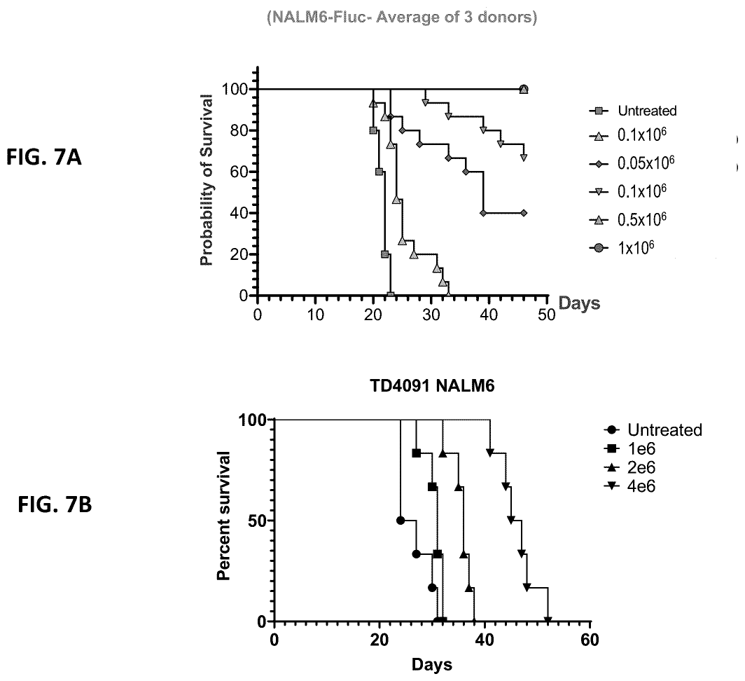

Anti-CD19 CAR T cells with TRAC, β2M, TGFBRII, and Regnase-1 knockouts (e.g., CTX112 cells) are further shown to be efficacious at low doses when infused into NSG mice bearing Nalm6-leukemia cells (see, FIGS. 7A-7B). Similar results were observed in xenograft mice models bearing Jeko-1 or Raji cells).

Overall, increase in mean survival was seen at even the lowest CTX112 doses tested (1×10⁴ CAR+ cells/mouse in the Nalm6 model and 5×10⁵ cells/mouse in the Jeko-1 and Raji models). In the majority of the groups across these studies (18 out of 20), the survival of the treated mice was significantly increased compared to survival of the untreated control (P<0.05, log-rank test). Increasing dose levels of CAR⁺ T cells showed a trend of improved tumor control in both the Raji and Jeko-1 models. Dose dependent tumor growth inhibition by CTX112 cells was observed in at least the Nalm6 mouse model.

### Example 8. Toxicology Study of CAR T Cells with Additional Edits

To evaluate whether the additional genomic edits present in the CAR-T cells (e.g., CTX112 cells) disclosed herein (i.e., TGFBR2 and Regnase-1 disruptions) increase the risk of cell activation in non-target tissues, an in vitro co-culture assay was conducted with primary human cell types from multiple lineages (heart, kidney, lung, bone, skeletal muscle, and blood) to represent major organ systems, and included CD19-expressing cells (peripheral blood mononuclear cells [PBMCs]).

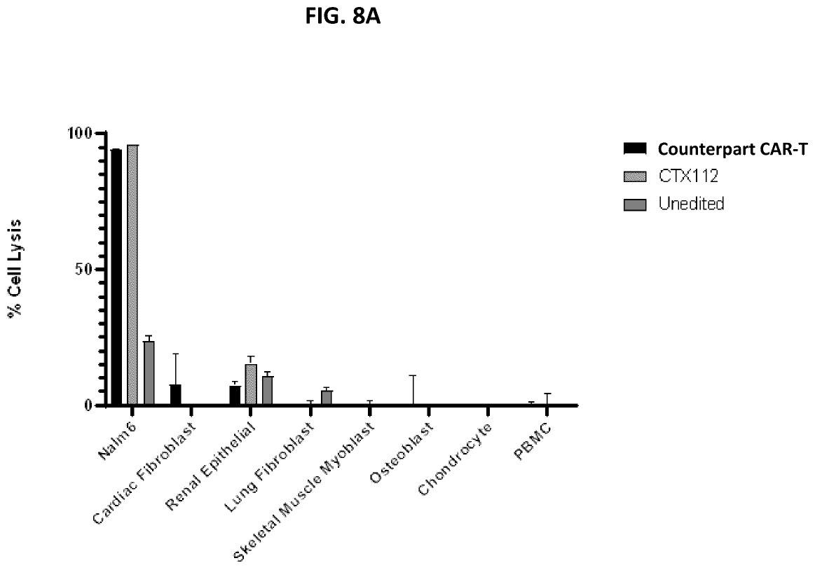

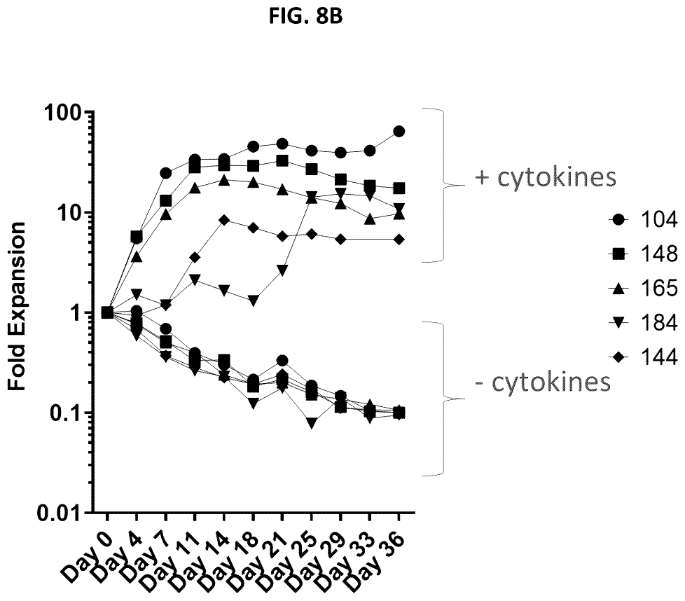

Activation of CTX112 cells was monitored by cytotoxicity assay based on luminescence (for adherent target cells) or flow cytometry (for non-adherent target cells). The CD19+ expressing cell line Nalm6 was used as a positive control for CTX112 activation. Cytotoxic activity of CTX112, the counterpart T cells having no edits in TGFBRII and Reg1, and unedited T cells was assessed following a 24-hour incubation at a 1:1 T cell to target cell ratio, with each of the target cells. As shown in FIG. 8A, high cytotoxic activity of both CTX112 and the counterpart cells lacking genetic edits in the TGFBRII and Regnase-1 genes was detected in the presence of the CD19+ Nalm6 control cell line, with lysis of over 90% of target cells after 24-hour co-culture (compared to ˜20% cell lysis when incubated with unedited T cells). Minimal cytotoxic activity was observed toward the other target cell types in the analyzed period, with either CTX112 or the counterpart T cells as disclosed herein.

To assess the ability of anti-CD19 CAR T cells with TRAC, β2M, TGFBRII, and Regnase-1 knockouts to grow in the absence of human cytokines, such T cells were produced from 5 donors and 10×10⁶ cells were placed in T cell media containing 5% human serum ±IL-2/IL-7.

The results show that although all preparations were able to grow in the presence of cytokines, none grew in the absence of cytokines (FIG. 8B). These data suggest that anti-CD19 CAR T cells with TRAC, β2M, TGFBRII, and Regnase-1 knockouts need cytokines for growth. Further, anti-CD19 CAR-T cells with TGFBRII and Regnase-1 disruptions (CTX112 cells) responded more robustly to both mouse and human cytokines when compared to the counterpart anti-CD19 CAR-T cells having no disruptions in TGFBRII and Regnase-1 genes, indicating that these additional edits make CTX112 cells better able to proliferate in response to immune stimulation.

### Example 9. Tumorigenicity Study in Immunocompromised Mice

To support the development of an allogeneic CAR T cell, a Good Laboratory Practice (GLP)-compliant tumorigenicity study in NSG mice was conducted. In particular, a 12-week, GLP-compliant study was conducted to evaluate the tumorigenic potential of anti-CD19 CAR T cells with TRAC, β2M, TGFBRII, and Regnase-1 knockouts following a single IV slow bolus injection in NOD/SCID/IL2Rγnull (NSG) mice after total body irradiation (total irradiation dose of 200 cGy) (Table 9). Dose volume was 250 μL/mouse for all groups. Radiation was delivered at a rate of 160 cGy/minute and targeted LD_{0/140}.

TABLE 9
GLP Tumorigenicity Study Design

| | Dose Level | Total Irradiation | Number of Animals | | |
|---|---|---|---|---|---|
| Test Article | (cells/mouse) | Dose (cGy) | Male | Female | Endpoints |
| Vehicle - no RT ^{a} | 0 | 0 | 5 | 5 | Clinical |
| Vehicle - RT ^{b} | 0 | 200 | 12 | 12 | observations, body |
| CD19 CAR + | 0.5 × 10⁶ | | 12 | 12 | weight, survival, |
| TRAC KO + β2M | | | | | hematology, serum |
| KO + R/T KO | | | | | chemistry, |
| CD19 CAR + | 1 × 10⁷ | | 12 | 12 | histopathology, |
| TRAC KO + β2M | | | | | exposure (ddPCR, |
| KO + R/T KO | | | | | IHC, cytokines) |

cGy: Centigray; ddPCR: droplet digital polymerase chain reaction; GLP: Good Laboratory Practice; IHC: immunohistochemistry; PBS: phosphate-buffered saline; RT: radiation treatment.
^{a} Animals were not irradiated and were not dosed with cells (administered PBS).
^{b} Animals were irradiated but were not dosed with cells (administered PBS).

Additional pathology and histopathological endpoints were assessed to evaluate the tolerability of the CAR T cells; however, because NSG mice do not produce B cells (the primary source of CD19 antigen), these results should be interpreted only as reflecting general off-target tolerability, with the knowledge that the CAR T cell expansion is not expected to occur in this model due to the absence of human CD19 antigen. The main endpoints of this study were survival, clinical observations, body weight measurements, and histopathology (see, Table 10).

The CAR T cell exposure in mouse blood was assessed by ddPCR and in tissues by immunohistochemistry.

TABLE 10
GLP Tumorigenicity Endpoints

| Endpoints | Assay Format | Time Points |
|---|---|---|
| Clinical observations | Cageside observations | 1-2x daily |
| Peripheral blood exposure | ddPCR | Days 7, 15, 29, |
| | | 43, 71, 84 |
| Histopathology | FFPE, H&E | Day 84 |
| Peripheral blood cytokines | Luminex (IL-2, IL-5, | Day 84 |
| (human cytokines) | IL-6, IL-8, IL-10, | |
| | IFN-γ, TNF-α, GM-CSF) | |
| Tissue exposure | IHC for hCD45 and hTCR | Day 84 |

ddPCR: droplet digital polymerase chain reaction; FFPE: formalin-fixed paraffin-embedded; GM-CSF: granulocyte-macrophage colony-stimulating factor; H&E: hematoxylin and eosin staining; hCD45: human CD45 protein; hTCR: human T cell receptor protein; IFN-γ: interferon gamma; IHC: immunohistochemistry; IL: interleukin; TNF-α: tumor necrosis factor alpha.

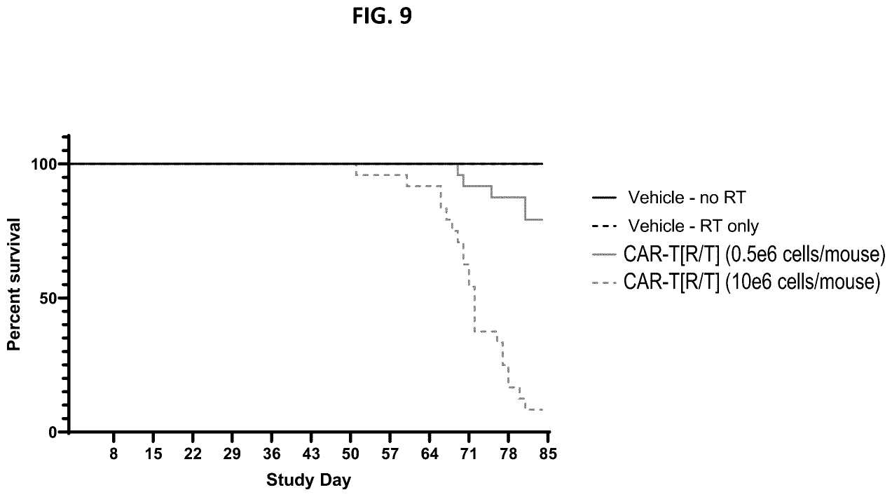

Mortality: There were 5/24 deaths (2/12 males and 3/12 females) in animals that were treated with a low dose of the CD19 CAR+TRAC KO+β2M KO+R/T KO T cells (0.5×10⁶ cells/mouse), with onset at Day 69. Mortality was also observed in animals treated with a high dose of the CAR T cells (1×10⁷ cells/mouse), where 12/12 males and 9/12 females were prematurely euthanized starting on Day 51, and 1/12 female was found dead on Day 66 (FIG. 9). Animals from the low-dose group were euthanized due to suspected bone fractures of the tail or hindlimb, hunched back, tucked abdomen, or difficulty with ambulation. Animals from the high-dose group were prematurely euthanized due to the aforementioned clinical signs, with additional findings of suspected bone fractures of the tail in 8/12 males and 4/9 euthanized females. Additional exhibited clinical signs from the high-dose group included but were not limited to: >20% body weight loss over a 1-week period, exudates around the eyes and nose, occasional respiratory distress (labored breathing), and decreased food and water consumption. Microscopic pathology revealed widespread mononuclear inflammation in all tissues examined. The cells in the mononuclear cell infiltrate often exhibited a positive reaction for hCD45 antigen and, to a lesser extent, hTCR antigen, indicating that they were derived from the infused human cells. All animals in both vehicle-treated groups survived to the scheduled necropsy at Day 84.

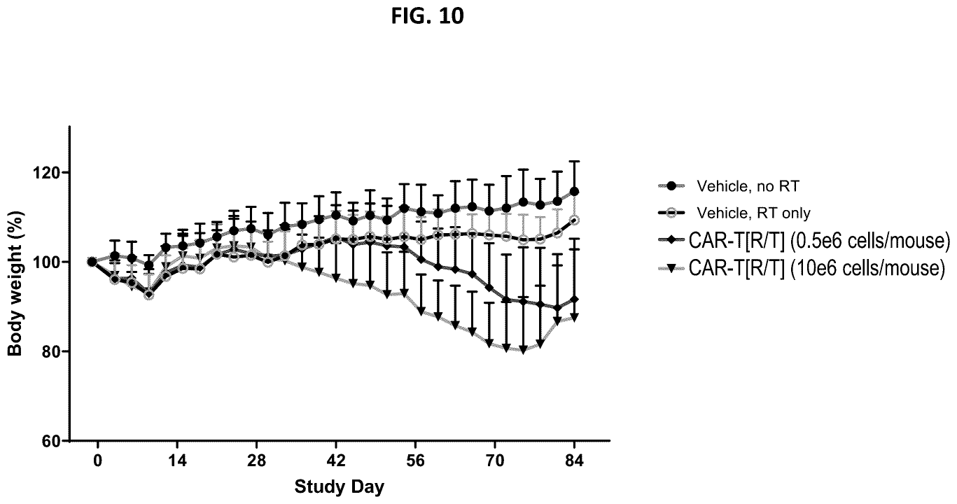

Clinical Observations: Starting on Day 32, nearly all animals treated with a low dose of the CD19 CAR+TRAC KO+β2M KO+R/T KO T cells (0.5×10⁶ cells/mouse) exhibited clinical signs, including: eye abnormalities (partly/fully closed and/or sunken), piloerection, hunched back, decreased activity, weakness, respiratory distress (labored breathing in 20/24 animals and/or increased rate in 11/24 animals), and lack of coordination. Additionally, occasional bone fractures were observed starting on Day 69 in either the hindlimb or tail in 11/24 animals. In the low-dose group, clinical signs were accompanied with body weight loss that warranted premature euthanasia of 5/24 animals between Days 69 and 81. Decreases in body weight changes were observed in animals treated with both the low dose (0.5×10⁶ cells/mouse) and high dose (1×10⁷ cells/mouse) (see, FIG. 10). There were no significant body weight changes in vehicle-treated animals.

Nearly all animals treated with the high dose (1×10⁷ cells/mouse) presented similar but more severe clinical signs with an earlier emergence. Starting on Day 6, animals exhibited hunched backs, followed by the aforementioned clinical signs of the low-dose group starting on Day 30. In addition, animals from the high-dose group exhibited suspected bone fractures in either the hindlimb or tail in 14/24 animals, tremors in 3/24 animals, and shallow respiration in 3/24 animals. These clinical signs were accompanied with severe body weight loss, leading to premature euthanasia of 22/24 animals between Days 51 and 80.

Clinical Pathology: Hematology was evaluated in animals that survived to scheduled necropsy on Day 84. Animals that received a low dose (0.5×10⁶ cells/mouse) saw a markedly higher total white blood cell count (48× in males, 36× in females) due to markedly higher neutrophil (12× in males and females) and lymphocyte (132× in males, 142× in females) counts, with minimal contributions (up to 2×) from large unstained cells and basophils when compared to the irradiated control group. Additional hematology parameters that were found to be up to 2-fold greater than in the irradiated control group included mildly higher reticulocyte counts, which resulted in minimally higher mean corpuscular volume, mildly higher red cell distribution width and hemoglobin distribution width as well as platelet distribution width.

Gross Pathology: Macroscopic observations associated with the administration of the CD19 CAR+TRAC KO+β2M KO+R/T KO T cells were present in the bone, lungs with bronchi, skin, and subcutis, and were considered related to inflammation compatible with GvHD. Enlarged spleen was also present in most animals that received a low or high dose (0.5×10⁶ cells/mouse or 1×10⁷ cells/mouse), which microscopically correlated with mild to marked infiltrate of mononuclear cells expressing moderate to severe reactivity for hCD45, and cells expressing minimal to marked reactivity for hTCR.

Microscopic Pathology: Microscopic observations of inflammation were present in the skin and subcutis, and/or lung, and/or bone with or without increased bone remodeling, and/or eye, and/or heart, and/or infusion/injection site, and/or nose, and or/tail, and/or anus of some animals that received either the low-dose (0.5×10⁶ cells/mouse) or the high-dose (1×10⁷ cells/mouse), with hematopoietic necrosis in 1 male that received the low-dose. Mononuclear cell infiltrate was present in the adrenal glands, bone marrow, brain, duodenum, large intestines, epididymides, esophagus, eyes, gall bladder, heart, infusion/injection site, kidneys, liver, lungs, various lymph nodes, mammary gland, nose, olfactory bulbs, optic nerves, ovaries, pancreas, pituitary gland, prostate gland, mandibular salivary gland, sciatic nerve, seminal vesicle, skeletal muscle (thigh), skin and subcutis, spinal cord (cervical, thoracic, lumbar), spleen, stomach, testes, thymus, thyroids, tongue, trachea, urinary bladder, uterus/cervix, and vagina of some animals that received the low- or high-dose. The cells in the mononuclear cell infiltrate often exhibited a positive reaction for hCD45 antigen and, to a lesser extent, hTCR antigen, indicating that they were derived from the infused human cells. No tumors or neoplastic lesions were found in any animals treated with the CD19 CAR+TRAC KO+β2M KO+R/T KO T cells.

Organ Weights: Organ weights were assessed from animals that survived to scheduled necropsy on Day 84. Organ weight increases related to the administration of the CD19 CAR+TRAC KO+β2M KO+R/T KO T cells were present in spleen, thymus, and adrenal glands from animals that received a low dose (0.5×10⁶ cells/mouse) and high dose (1×10⁷ cells/mouse) the CAR T cells compared to irradiated control animals and were considered to be related to mononuclear cell infiltrate due to the CAR T cell engraftment.

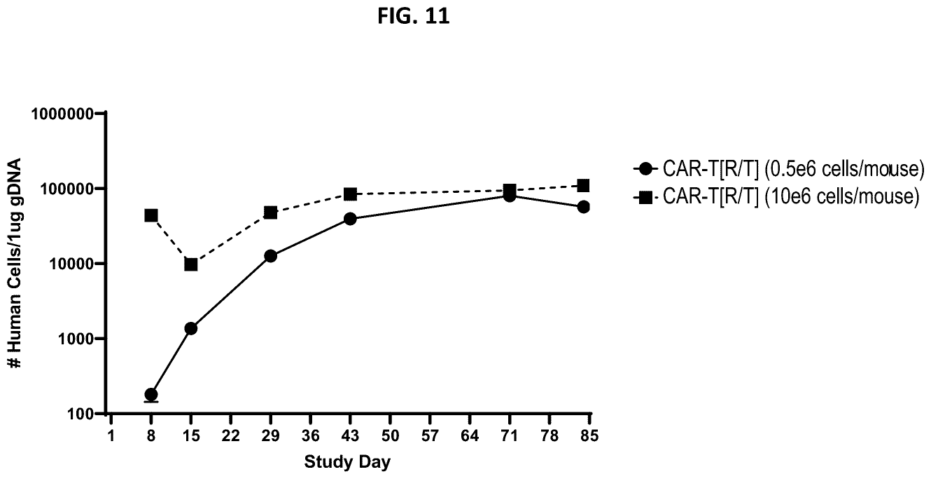

CAR T Cell Exposure in Blood: Circulating CAR T cells were detected by ddPCR at the first time point assessed (Day 8 after administration) in 12/17 animals treated with the low dose and in 19/19 animals treated with the high dose (FIG. 11). At Day 15, circulating CAR T cells were detected in all assayed animal samples and maintained through the course of the study.

An additional toxicity study was conducted to assess the safety and tumorigenicity of the CD19 CAR+TRAC KO+β2M KO+R/T KO T cells at lower doses (5×10³ and 1×10⁵ cells/mouse). The study duration was increased to 20 weeks to enable monitoring of full CAR T cell exposure based upon persistence beyond 12 weeks observed in the initial study. The results of this additional study demonstrate that toxicity can be mitigated by lowering the administered dose, and that there are dose levels that control tumor growth and also exhibit minimal toxicity.

### Example 10. In Vitro Mixed Lymphocyte Reaction Study

The interaction between the anti-CD19 CAR T cells with TRAC, β2M, TGFBRII, and Regnase-1 knockouts and PBMCs were assessed in MLR assays. The CAR T cells were co-cultured with PBMCs and/or subsets, and proliferation or cytotoxicity of allogeneic cells were assessed in vitro compared to co-cultures established with the CAR T cells.

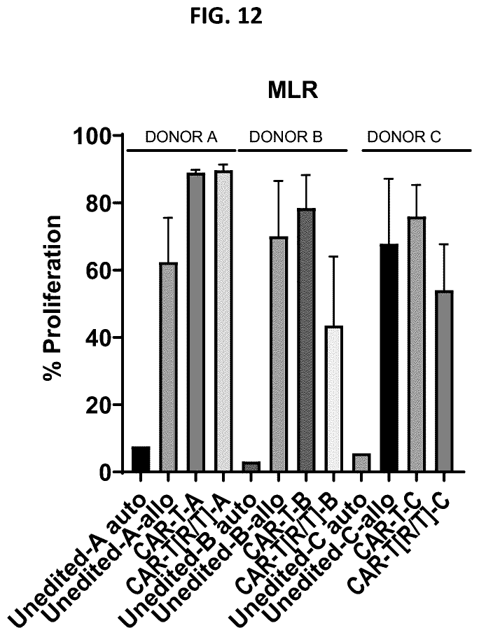

The results are shown in FIG. 12. Comparable allogeneic MLR responses were exhibited in the anti-CD19 CAR T cells with TRAC and β2M knockouts, with or without the additional knockouts of TGFBRII and Regnase-1.

### Example 11. In Vitro Natural Killer Cell Rejection

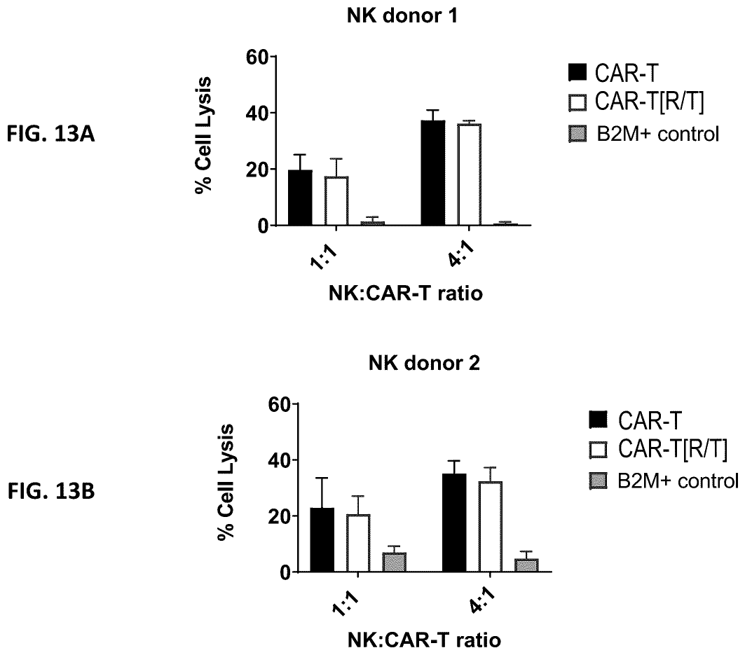

To assess the ability of allogeneic NK cells to lyse the anti-CD19 CAR T cells, the CAR T cells with TRAC and β2M knockouts and with or without the additional knockouts of TGFBRII and Regnase-1 as well as β2M⁺ control cells (CAR T cells that were not edited) from 3 unique healthy donors were co-cultured with allogeneic NK cells from 2 unique donors overnight and subjected to a flow cytometry-based cytotoxicity assay. The percentages of lysed T cells were shown in FIG. 13.

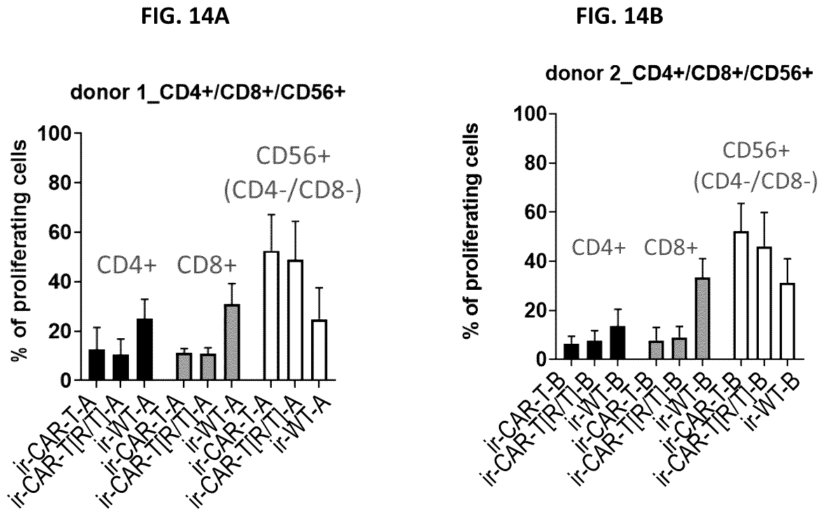

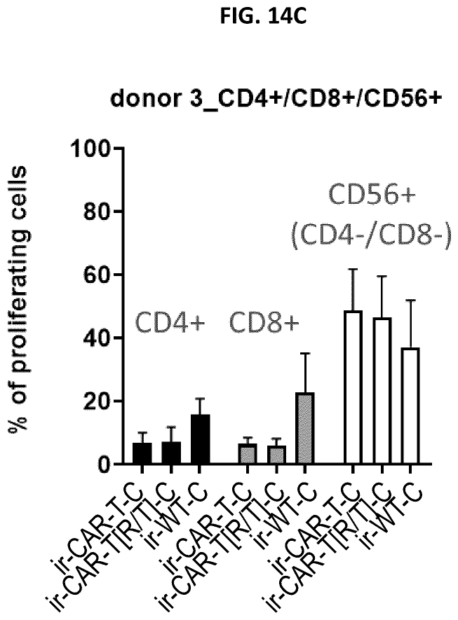

The results show that the anti-CD19 CAR T cells with TRAC and β2M knockouts and without R/T knockouts (β2M-negative average 66%) and the anti-CD19 CAR T cells with TRAC, β2M, and R/T knockouts (β2M-negative average 64%) were shown to be comparably lysed by NK cells. Control allogeneic cells from the same healthy donors as the CAR T cells that were not edited (β2M⁺) were not lysed at similar levels. The CAR T cells with the additional R/T knockouts did not show any appreciable resistance to NK-mediated attack relative the CAR T cells without the additional R/T knockouts. Additionally, the CAR T cells with or without the additional R/T knockouts showed comparable allogeneic T cell responses as well (see, FIGS. 14A-14B).

### Example 12. A Phase 1/2, Open-Label, Multicenter, Dose Escalation and Cohort Expansion Study of the Safety and Efficacy of Anti-CD19 Allogeneic CRISPR-Cas9-Engineered T Cells (CTX112) in Subjects with Relapsed or Refractory B Cell Malignancies

This is an open-label, multicenter, Phase 1/2 study evaluating the safety and efficacy of CTX112 in adult subjects with relapsed/refractory B cell malignancies.

### 1. Investigational Product

CTX112 is a CD19-directed allogeneic T cell immunotherapy consisting of T cells that are genetically modified using CRISPR-Cas9 (clustered regularly interspaced short palindromic repeats-CRISPR-associated protein 9) gene editing components (single-guide RNA and Cas9 nuclease). Genetic modifications involved in CTX112 cells are listed below:
Disruption of the T cell receptor alpha constant (TRAC) region gene to eliminate surface expression of T cell receptor (TCR) alpha and beta chains and thus its interactions with the host major histocompatibility system (MHC) system to reduce the probability of graft vs host disease (GvHD)
Disruption of the beta-2 microglobulin (B2M) gene to eliminate MHC class I surface expression and increase CAR T cell persistence by reducing the probability of host rejection
Insertion of a CD19-targeting CAR (comprising the amino acid sequence of SEQ ID NO: 72) into the TRAC gene locus at the site of SEQ ID NO: 18 via an adeno associated virus (AAV) expression cassette to direct the CAR T cells towards CD19-expressing tumor cells.
Disruption of the transforming growth factor beta receptor 2 (TGFBR2) gene to eliminate surface expression of TGFBR2 and reduce the immunosuppressive effect of transforming growth factor beta (TGF-β)
Disruption of the regulatory RNase 1 (Regnase-1) gene to improve functional persistence of CAR T cells (see Section 1.6.2)

CTX112 cells are prepared from healthy donor peripheral blood mononuclear cells obtained via a standard leukapheresis procedure. The modified T cells are expanded in cell culture, washed, and formulated into a suspension comprising human serum albumin in normal saline and a cryopreservation solution, which is then cryopreserved. The product is thawed prior to administration.

CTX112 cells for clinical uses contain ≤0.15% TCR⁺ cells and ≥30% CAR⁺ T cells.

### 2. Study Objectives and Endpoints

The study objectives and endpoint of this example is summarized in Table 11 below:

TABLE 11
Study Objectives and Endpoints

| Objectives | Endpoints |
|---|---|
| Primary Objective | Primary Endpoint |
| Phase 1 - Part A (dose escalation) and Part B (dose | Phase 1 - Parts A and Part B |
| optimization in disease-specific cohorts) | Incidence of AEs defined as DLTs |
| To assess the safety of escalating doses of | |
| CTX112 in combination with LD | |
| chemotherapy in subjects with | |
| relapsed/refractory B cell malignancies to | |
| determine the recommended Phase 2 dose(s) | |
| Phase 2 (expansion of selected Part B cohorts) | Phase 2 |
| To assess the efficacy of CTX112 in | The ORR (CR + PR) per the Lugano |
| combination with LD chemotherapy in selected | Response Criteria for Malignant Lymphoma |
| Part B cohorts at the recommended Phase 2 | or iwCLL 2018 Guidelines for CLL/SLL |
| dose determined in Parts A and B | |
| Secondary Objectives (Phase 1 and Phase 2) | Secondary Endpoints (Phase 1 and Phase 2) |
| To further characterize the efficacy, safety, | Efficacy |
| and pharmacokinetics of CTX112 | For Phase 1 - Parts A and B, the |
| | objective response rate (CR + PR) per the |
| | Lugano Response Criteria for Malignant |
| | Lymphoma or iwCLL 2018 Guidelines for |
| | CLL/SLL |
| | Duration of response/remission |
| | Duration of clinical benefit |
| | Treatment-failure-free survival |
| | Progression-free survival |
| | Overall survival |
| | Time to response |
| | Safety |
| | Frequency and severity of AEs and |
| | clinically significant laboratory |
| | abnormalities |
| | Pharmacokinetic |
| | Levels of CTX112 in blood over time |
| Exploratory Objectives (Phase 1 and Phase 2) | Exploratory Endpoints (Phase 1 and Phase 2) |
| To identify biomarkers associated with | Levels of CTX112 in tissues (trafficking |
| CTX112 that may indicate or predict clinical | of CTX112 in bone marrow, CSF, and/or |
| response, resistance, safety, or | tumor tissue may be evaluated in any |
| pharmacodynamic activity | samples collected per protocol-specific |
| | sampling) |
| | Levels of cytokines in blood. Levels in |
| | other tissues may additionally be evaluated |
| | Incidence of anti-CTX112 antibodies |
| | Levels of B cells and Igs over time |
| | Impact of anti-cytokine therapy on |
| | CTX112 proliferation, CRS, and response |
| | Phase 1 Part B and Phase 2 only: For |
| | CLL/SLL subjects only, percentage of |
| | subjects who are negative for MRD |
| To characterize outcomes to subsequent | Incidence and outcomes of autologous or |
| therapies for patients who progress or relapse | allogeneic HSCT following CTX112 |
| after CTX112 | therapy |
| | Incidence and type of subsequent |
| | anticancer therapy |
| | First subsequent therapy-free survival |
| Phase 2 Only: To evaluate the changes | Phase 2 Only: Change over time in |
| over time in patient-reported outcomes | PROs |
| associated with CTX112 | |

AE: adverse event; CLL: chronic lymphocytic leukemia; CR: complete response; CRS: cytokine release syndrome; DLT: dose-limiting toxicity; Ig: immunoglobulin; iwCLL: International Workshop on Chronic Lymphocytic Leukemia; HSCT: hematopoietic stem cell transplant; LD: lymphodepleting; MRD: minimal residual disease; ORR: objective response rate; PR: partial response; PRO: patient-reported outcome; relapsed/refractory:relapsed or refractory; SLL: small lymphocytic leukemia

### 3. Subject Eligibility

Inclusion Criteria
1. Age ≅18 years.
2. Able to understand and comply with protocol-required study procedures and voluntarily sign a written informed consent document.
3. Refractory or relapsed B cell malignancy in 1 of the following disease types based on histology shown in Table 12 below:

TABLE 12
Histology of B Cell Malignancies

| Disease Type (Tumor Histology | |
|---|---|
| and Prior Treatments) | Eligibility Criteria |
| FL, grade 1-3a | At least 1 of the following: |
| | a. Progressed after at least 2 lines of systemic therapy, including |
| | treatment with anti-CD20 antibody-based regimen (e.g., R- |
| | bendamustine, R-CHOP) |
| | b. Early relapse, defined as recurrence or progression of disease |
| | within 24 months of front-line treatment (POD24) |
| MZL | Relapsed and/or refractory disease after up to 5 prior lines of |
| | therapy; prior treatment must include an anti-CD20 antibody- |
| | based regimen |
| MCL | Relapsed and/or refractory disease after up to 5 prior lines of |
| | therapy; must include prior anthracycline- or |
| | bendamustine-containing chemotherapy, an anti-CD20 |
| | monoclonal antibody, or a BTK inhibitor |
| CLL/SLL | Progressed after at least 2 prior therapies and prior treatment with, |
| | or intolerance of, both a BTK inhibitor (e.g., ibrutinib) and |
| | venetoclax |
| High-grade LBCL: DLBCL NOS, | Prior therapy must include: |
| high-grade B cell lymphoma with | a. 2 or more lines of prior therapy, including an anti-CD20 |
| MYC and BCL2 and/or BCL6 | monoclonal antibody and an anthracycline-containing regimen. |
| rearrangements, grade 3b FL, | Subjects who have received autologous HSCT must have |
| transformed FL, transformed MCL, | recovered from HSCT-related toxicities |
| or transformed MZL; For Phase 2, | b. For subjects with transformed FL, subjects must have received |
| also PMBCL | at least 1 line of chemotherapy for disease after transformation to |
| | DLBCL |
| DLBCL with prior CAR T treatment | All of the following:¹ |
| (Phase 1 Part B/Phase 2 only) | a. Received prior treatment with FDA-approved autologous |
| | CD19-directed CAR T cell therapy (i.e., axi-cel, tisa-cel, |
| | liso-cel) ≥12 months prior to enrollment |
| | b. Achieved a CR for ≥4 months duration from that prior |
| | treatment |
| | c. No elevated LDH |
| | d. Age <70 years old |

BTK: Bruton tyrosine kinase; CAR: chimeric antigen receptor; CLL: chronic lymphocytic leukemia; CR: complete response; DLBCL: diffuse large B cell lymphoma; FDA: Food and Drug Administration; FL: follicular lymphoma; HSCT: hematopoietic stem cell transplant; LDH: lactate dehydrogenase; MCL: mantle cell lymphoma; MZL: marginal zone lymphoma; NOS: not otherwise specified; PMBCL: primary mediastinal large B cell lymphoma; R-CHOP: chemoimmunotherapy consisting of rituximab, cyclophosphamide, doxorubicin, vincristine, and prednisone; SLL: small lymphocytic leukemia
¹Subjects with relapse after autologous CAR T cell therapy <6 months and/or 6 to 12 months prior to enrollment, those with elevated LDH, and those >70 years old may be considered after safety and efficacy data of at least 6 patients dosed in the initial cohort are reviewed.

4. For all disease subtypes, histologically confirmed and measurable disease based on:
   a. Confirmation of tumor histology from local pathology lab (archival tissue from last relapse/progression [within 3 months of enrollment] or biopsy during screening [or within 28 days prior to CTX112 infusion, if a recent sample was collected as part of standard of care]).
   b. At least 1 measurable lesion that is fluorodeoxyglucose positron emission tomography (PET)-positive, as defined by Lugano criteria (Deauville score of 4 or 5 on Lugano criteria 5-point scale). Note: Previously irradiated lesions will be considered measurable only if progression of the irradiated lesion(s) is documented following completion of radiation therapy.
   c. For MCL, confirmation of cyclin D1 overexpression or presence of translocation t(11; 14).
   d. For CLL/SLL, diagnosis of CLL with indication for treatment based on the iwCLL 2018 guidelines and clinically measurable disease or SLL with measurable disease that is biopsy-proven.
5. Eastern Cooperative Oncology Group (ECOG) performance status 0 or 1.
6. Meets criteria to undergo LD chemotherapy and CAR T cell infusion.
7. Adequate organ function:
   a. Renal: Estimated glomerular filtration rate >50 mL/min/1.73 m².
   b. Liver: Aspartate transaminase (AST) or alanine transaminase (ALT)≤3× upper limit of normal (ULN); total bilirubin <2.0 mg/dL (for subjects with Gilbert's syndrome or lymphomatous infiltration of the liver, total bilirubin <3.0 mg/dL).
   c. Cardiac: Hemodynamically stable and left ventricular ejection fraction ≥45% by echocardiogram.
   d. Pulmonary: Oxygen saturation level on room air >91% per pulse oximetry.
   e. Hematological: No grade 4 neutropenia and at least 2 weeks from last growth factor support injection. No grade 4 thrombocytopenia and at least 1 week from last platelet transfusion.
8. Female subjects of childbearing potential (postmenarcheal with an intact uterus and at least 1 ovary, who are less than 1 year postmenopausal) must agree to use acceptable method(s) of contraception from enrollment through at least 12 months after the most recent CTX112 infusion.
9. Male subjects must agree to use effective acceptable method(s) of contraception from enrollment through at least 12 months after the most recent CTX112 infusion.

### Exclusion Criteria

1. Prior allogeneic HSCT
2. Treatment with the following therapies:
   a. Prior treatment with any gene therapy or genetically modified cell therapy Exception: Subjects with DLBCL who have received prior anti-CD19 autologous CAR T cell therapy may be enrolled in a designated Part B cohort, which could expand in Phase 2.
   b. Prior treatment with a CD19-directed therapy, including CD19-directed antibody, bispecific T cell engager, or antibody-drug conjugate. For subjects meeting inclusion criteria for DLBCL/high-grade B cell lymphoma: Prior CD19-directed treatment may be allowed if there is confirmed CD19 expression (by immunohistochemistry [IHC] or flow cytometry) after progression or relapse following most recent CD19-directed treatment.
3. For subjects with prior immunotherapy (i.e., bispecific T cell engagers or CAR T cells, if applicable):
   a. History of grade ≥2 ICANS from prior immunotherapy
   b. History of grade 4 CRS
   c. Any other prior or residual toxicity from prior immunotherapy that would place the subject at increased risk of toxicity from a subsequent CAR T therapy
4. Subjects with Richter's transformation.
5. Diagnosis of Burkitt's lymphoma/leukemia.
6. Known contraindication to cyclophosphamide, fludarabine, or any of the excipients of CTX112 product.
7. Life expectancy of less than 6 weeks.
8. Detectable malignant cells from CSF or magnetic resonance imaging (MRI) indicating brain metastases during screening, or a history of central nervous system (CNS) involvement by malignancy (CSF or imaging).
9. History of a seizure disorder, major cerebrovascular ischemia/hemorrhage, dementia, cerebellar disease, or any autoimmune disease with CNS involvement.
10. Unstable angina, clinically significant arrhythmia, or myocardial infarction within 6 months of enrollment, or grade ≥3 pericardial effusion at the time of enrollment.
11. Presence of active bacterial, viral, or fungal infection that is uncontrolled.
12. Positive for presence of human immunodeficiency virus (HIV) type 1 or 2, or active hepatitis B virus (HBV) or hepatitis C virus (HCV) infection. Subjects with prior history of HBV or HCV infection who have documented undetectable viral load (by quantitative polymerase chain reaction [PCR] or nucleic acid testing) are permitted.
13. Previous or concurrent malignancy, except basal cell or squamous cell skin carcinoma, adequately resected and in situ carcinoma of cervix, or a previous malignancy that was completely resected and has been in remission for ≥5 years of enrollment.
14. Radiation therapy within 7 days prior to enrollment.
15. Use of systemic antitumor therapy (including steroids) or investigational agent within 14 days or 5 half-lives whichever is longer, prior to enrollment.
   Exceptions: 1) prior inhibitory/stimulatory immune checkpoint molecule therapy, is allowed within 3 half-lives prior to enrollment; 2) rituximab, other anti-CD20 monoclonal antibodies, or polatuzumab vedotin is allowed within 30 days prior to enrollment (however, PET/computed tomography (CT) scan needs to occur at least 2 weeks after last dose); and 3) for subjects with MCL only, a BTK inhibitor within 7 days prior to LD chemotherapy is allowed.
16. Primary immunodeficiency disorder or active autoimmune disease requiring steroids and/or other immunosuppressive therapy.
17. Diagnosis of significant psychiatric disorder or other medical condition that could impede the subject's ability to participate in the study.
18. Women who are pregnant or breastfeeding.

### 4. Study Design

This is an open-label, multicenter, Phase 1 study evaluating the safety and efficacy of CTX112 in adult subjects with relapsed/refractory B cell malignancies.

Investigational Plan

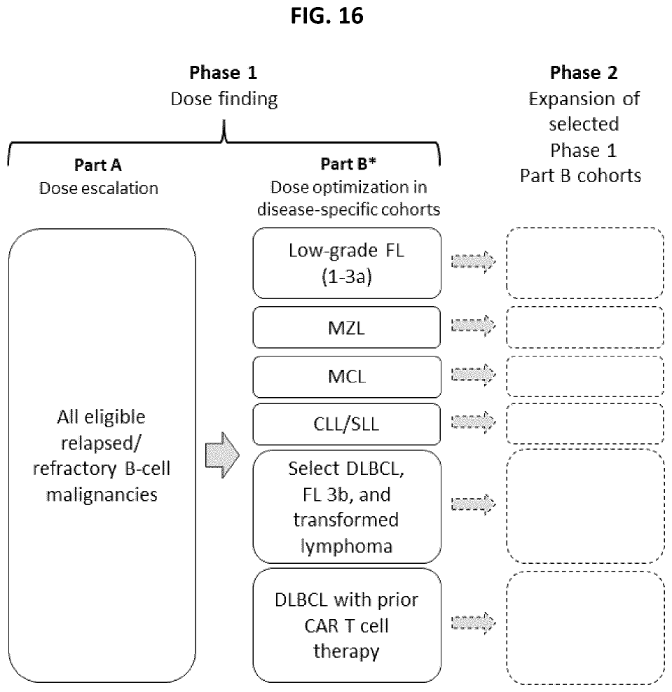

The study is divided into 2 phases: Phase 1 for dose escalation (Part A) and dose optimization in disease-specific cohorts as determined by histology or prior treatments (Part B) followed by Phase 2 for expansion of 1 or more Phase 1 Part B cohorts (see, FIG. 16). Any or all Phase 1 Part B disease-specific cohorts may enroll. In addition, cohorts in Phase 1 Part B may start enrolling at or below the highest dose level cleared in Part A and before Phase 1 Part A is complete. Select subtypes of large B cell lymphoma include DLBCL not otherwise specified and high-grade B cell lymphoma with MYC and BCL2 and/or BCL6 rearrangements, primary mediastinal large B cell lymphoma (Phase 2 only), transformed FL and grade 3b FL, transformed MCL, or transformed MZL.

The study consists of 3 main stages as follows:
Screening to determine eligibility for treatment (up to 14 days).
LD chemotherapy and infusion of CTX112.
   LD chemotherapy consisting of co-administration of fludarabine 30 mg/m²+cyclophosphamide 500 mg/m² IV daily for 3 days; both agents may be started on the same day and administered for 3 consecutive days and completed at least 48 hours (but no more than 7 days) prior to CTX112 infusion.
   CTX112 infusion.
Follow up for all cohorts (up to 5 years after the last CTX112 infusion).

In Phase 1, dose escalation (Part A) investigates escalating doses of CTX112 in subjects with any of the eligible lymphoma and leukemia disease histology. A maximum of 2 subjects of the same disease histology are enrolled at each dose level. Part B (dose optimization) assess safety and preliminary efficacy in selected disease-specific cohorts. Part B cohorts may enroll concurrently with Part A. Multiple dose levels may be evaluated in Part B (up to 9 subjects per dose level within each Part B cohort), with the starting dose level at or below any dose level cleared in Part A. If a maximum tolerated dose (MTD) is reached in Part A, individual Part B cohorts may explore higher dose levels under certain conditions described in the Dose Optimization in Disease-specific Cohorts section below.

Phase 2 involves expansion (n≤20) for 1 or more Part B cohorts at the recommended Phase 2 dose(s) determined in Part A and Part B.
Subjects participate in this study for up to 5 years after the last CTX112 infusion. After completion of this study, all subjects are asked to participate in a separate long-term follow-up study for an additional 10 years to assess long-term safety and survival.

Phase 1: CTX112 Dose Escalation

Part A: Dose Escalation

Table 13 presents the doses of CTX112, based on the total number of CAR⁺ T cells, that may be evaluated in this study. There is a dose limit of 7×10⁴ TCR⁺ cells/kg per CTX112 infusion for all dose levels. Based on the percentage of CAR⁺ T cells in the CTX112 lot to be administered, enrollment at higher dose levels (e.g., DL4) may be restricted to subjects with a minimum weight to ensure the TCR⁺ cell limit is not exceeded.

TABLE 13
Planned CTX112 Dose Levels

| Dose Level | Total CAR⁺ T Cell Dose |
|---|---|
| −1 (de-escalation) | 1 × 10⁷ |
| 1 | 3 × 10⁷ |
| 2 | 1 × 10⁸ |
| 2.5 | 2 × 10⁸ |
| 3 | 3 × 10⁸ |
| 3.5 | 4.5 × 10⁸ |
| 4 | 6 × 10⁸ |

CAR: chimeric antigen receptor; DL: Dose Level.

Dosing begins at Dose Level 1 and escalation occurs in half-log (DL2 and DL3) or 2-fold (DL4) increments. Following review of the clinical data, intermediate dose levels (i.e., DL2.5 and DL3.5) may be explored in escalation or de-escalation contingent on the cumulative safety, efficacy, and PK data generated at previous dose levels. If an intermediate dose level is selected, further dose escalation will be capped at a half-log increment from the highest cleared dose.

In Part A, dose escalation is performed using the Bayesian Optimal Interval Design (BOIN), calibrated with a target DLT rate of 30% for the MTD, an escalation boundary of 25%, and a de-escalation boundary of 35% based on the observed DLT rate. The following provisions apply:
A minimum of 3 subjects are evaluated for each dose escalation decision
If a DLT occurs in the first 3 subjects at any dose level, a minimum of 6 subjects are evaluated for the dose escalation decision at that dose level
A maximum of 30 subjects are evaluated in Part A across all dose levels
A cap of 1 to 3 subjects of any single disease histology at each dose level may be imposed to ensure that a variety of disease types are explored in Part A

The DLT evaluation period begins with CTX112 infusion and lasts for 28 days. The first subject is dosed with CTX112 at DL1 (3×10⁷ CAR⁺ T cells) (Table 13). The first 3 subjects enrolled in the study (e.g., Dose Level 1) are treated in a staggered manner such that each subject only receives CTX112 once the preceding subject has completed the DLT evaluation period (i.e., staggered by 28 days). Based on the observed toxicity profile at Dose Level 1 (e.g., no DLTs), a 28-day staggering interval applies to the first 2 subjects in Dose Level 2 and above. Additional subjects at each dose level may be enrolled and dosed concurrently.

Part B: Dose Optimization in Disease-Specific Cohorts

In Part B, subjects are enrolled into specific cohorts as determined by disease histology to inform the dose selection for Phase 2 (FIG. 16). Part B cohorts open and may enroll concurrently with Part A. The number of subjects enrolled to a given dose level for Part B cohorts is determined on an ongoing basis based on cumulative safety, efficacy, and applicable PK data. Up to 9 subjects may be enrolled to each dose level for any Part B cohort.

The starting dose for each Part B cohort must be at or below any dose level already cleared in Part A. Additional dose levels cleared in Part A may be explored within each Part B cohort for dose optimization.

However, if an MTD is reached in Part A, individual Part B cohorts may continue to explore higher dose levels if the following two conditions are met, combining data from Parts A and B:
At least 3 subjects in that disease-specific cohort have been treated at a given dose level across Part A and Part B (e.g., the MTD established in Part A), and
An escalation decision is recommended by the BOIN design method, with a target DLT rate and boundaries as described for Part A.

Conversely, if a dose level is cleared in Part A, but emerging safety data in a Part B cohort show higher toxicity at that dose, BOIN may also be applied to determine a safe dose for that disease-specific cohort. If de-escalation occurs in a Part B cohort, this does not impact enrollment for other disease histologies in Part A or in other Part B cohorts. Final decisions whether to enroll subjects to a higher/lower dose level or eliminate a dose from consideration shall be made in accordance with the BOIN recommendations.

The rationale for Part B is that it enables CTX112 dosing to be optimized for each disease histology. Although the occurrence of DLTs, if observed, is expected to be similar across these disease subtypes, some previous studies have shown that response to autologous CAR T cell therapy may vary by disease histology, as may the incidence and severity of CRS and ICANS (BREYANZI USPI, 2021; Jacobson et al., 2020; KYMRIAH USPI, 2020; Siddiqi et al., 2019; Wang et al., 2020; YESCARTA USPI, 2021, 2022). The design of Part B allows cumulative safety and efficacy data to inform dose selection for each specific disease histology in this study.

Bayesian Optimal Interval Design (BOIN) Method

The BOIN method is a model-assisted approach which provides prespecified decision rules that recommend escalating, de-escalating, or maintaining the same dose or stopping the dose escalation based on the number of subjects with DLTs observed in the dose level under evaluation (Liu S, 2015)

The BOIN method is applied in Part A and within each disease-specific cohort in Part B, where the dose rules are calibrated with a target DLT rate of 30% for the MTD and an escalation boundary of 25% and de-escalation boundary of 35% based on the observed DLT rate. Detailed steps to implement the BOIN method as well as to identify the MTD following conventional practice.

Maximum Tolerated Dose Definition

In Part A, the MTD is determined by methods described above using Part A data only. In Part B, the disease-specific MTD is determined by the same methods, taking into account data from both Part A and Part B. An MTD may not be determined in this study. However, a decision to move to the Phase 2 expansion of disease-specific cohorts may be made in the absence of an MTD.

DLT Definitions

The DLT evaluation period begins with CTX112 infusion and lasts for 28 days. Subjects must receive CTX112 to be evaluated for a DLT. If a subject discontinues the study any time prior to CTX112 infusion, the subject will not be evaluated for DLT and a replacement subject will be enrolled at the same dose level as the discontinued subject. If a DLT-evaluable subject has signs or symptoms of a potential DLT, the DLT evaluation period will be extended according to the protocol-defined window to allow for improvement or resolution before a DLT is declared.

Toxicities are graded and documented according to National Cancer Institute Common Terminology Criteria for Adverse Events (CTCAE) version 5.0, with the following exceptions:

CRS:
American Society for Transplantation and Cellular Therapy (ASTCT) criteria (Lee et al., 2019, Biol Blood Marrow Transplant 25, 625-638)

Neurotoxicity:
CTCAE v5.0
ICANS criteria (Lee et al., 2019)

GvHD:
Mount Sinai Acute GVHD International Consortium (MAGIC) criteria (Harris et al., 2016, Biol Blood Marrow Transplant 22, 4-10).

AEs that have no plausible causal relationship with CTX112 are not considered DLTs. A DLT is defined as any of the following CTX112-related events occurring during the DLT evaluation period that persist beyond the specified duration (relative to the time of onset):
1. Any death without clinical or radiologic evidence of disease progression
2. Cytokine release syndrome (CRS): Grade 4 CRS, or any grade 3 CRS that fails to improve to grade ≤2 within 72 hours, per ASTCT criteria
3. ICANS: Grade 3 or higher events of neurotoxicity/ICANS, per ASTCT criteria, of any duration
4. Acute GvHD: Grade 3 or higher acute GvHD of any duration; grade 2 acute GvHD that is steroid refractory, defined as progression after 3 days of steroid treatment (e.g., 1 mg/kg/day), or no response after 7 days of steroid therapy
5. Grade 4 neutropenia or thrombocytopenia, not attributable to underlying disease, that does not improve to grade ≤3 within 42 days. Grade 3 thrombocytopenia accompanied by clinically significant (grade 2 or higher) bleeding
6. Excessive occurrence of grade 3 or higher infections are assessed as follows: all grade 3 or higher infections occurring within 3 months following treatment with CTX112 are assessed retrospectively. After at least 6 subjects are administered CTX112, if ≥35% of subjects experience grade 3 or higher infections (bacterial, viral, and/or fungal) in the absence of clinical or radiological evidence of disease progression, dose escalation are suspended
7. Grade ≥3 toxicity involving vital organs (e.g., cardiac, pulmonary) of any duration
8. All other grade 3 or higher toxicities that are clinically significant, without clinical or radiologic evidence of disease progression, and that do not resolve to grade ≤2 within 72 hours except as listed below.

The following are NOT considered DLTs:
a. Grade 3 or 4 fever
b. Grade 3 or 4 febrile neutropenia that resolves within 72 hours
c. Grade ≥3 allergic reaction improving to grade ≤2 within 48 hours of instituting supportive care
d. B cell aplasia and/or hypogammaglobulinemia
e. Grade 3 or 4 abnormal hepatic function tests (i.e., elevated aspartate aminotransferase [AST] or alanine aminotransferase [ALT]) that improve to grade ≤2 within 7 days
f. Grade 3 or 4 abnormal renal function studies (e.g., elevated creatinine) that improve to grade ≤2 within 7 days
g. Grade ≤3 tumor lysis syndrome (TLS) lasting ≤7 days

AEs that have no plausible causal relationship with CTX112 are not considered DLTs.

Subjects must receive CTX112 to be evaluated for a DLT. If a subject discontinues the study any time prior to CTX112 infusion, the subject is not evaluated for DLT and a replacement subject is enrolled at the same dose level as the discontinued subject. If a DLT-evaluable subject has signs or symptoms of a potential DLT for which the protocol definition allows time for improvement or resolution, the DLT evaluation period is extended accordingly before a DLT is declared.

Phase 2: CTX112 Cohort Expansion

Phase 2 involves expansion (n≤20) for 1 or more Part B cohorts at the recommended Phase 2 dose determined in Part A and Part B in order to evaluate the safety and efficacy of the recommended Phase 2 dose(s).

### 5. Study Treatment

Lymphodepleting Chemotherapy

All subjects receive LD chemotherapy prior to CTX112 infusion. LD chemotherapy consists of:
Fludarabine 30 mg/m² IV daily for 3 doses AND
Cyclophosphamide 500 mg/m² IV daily for 3 doses.

Both LD agents are started on the same day and administered for 3 consecutive days. Subjects should start LD chemotherapy within 7 days after study enrollment. Adult subjects with moderate impairment of renal function (creatinine clearance 30-70 mL/min/1.73 m²) may receive a reduced dose of fludarabine in accordance with applicable prescribing information.

Reference the current full prescribing information for fludarabine and cyclophosphamide for guidance regarding the storage, preparation, administration, supportive care instructions, and toxicity management associated with LD chemotherapy.

LD chemotherapy before the CTX112 infusion is delayed if any of the following signs or symptoms are present:
Significant worsening of clinical status that increases the potential risk of AEs associated with LD chemotherapy
Requirement for supplemental oxygen to maintain a saturation level of >91%
New uncontrolled cardiac arrhythmia
Hypotension requiring vasopressor support
Active infection: Positive blood cultures for bacteria, fungus, or virus not responding to treatment, or negative culture but active infection
Grade ≥2 acute neurological toxicity

During Parts A or B, if LD chemotherapy is delayed more than 30 days or the subject starts anticancer therapy, the subject is replaced if deemed required for dose escalation decisions. For subjects whose toxicity(ies) are driven by underlying disease and require anticancer therapy, they must subsequently meet disease inclusion criteria, treatment washout, and end-organ function criteria before restarting LD chemotherapy. Additionally, any subject who received anticancer therapy after enrollment (besides LD chemotherapy) must have disease evaluation (including PET/CT scan) performed prior to starting LD chemotherapy.

Administration of CTX112

CTX112 consists of allogeneic T cells modified with CRISPR-Cas9, resuspended in cryopreservative solution and supplied in a 6-mL infusion vial. A flat dose of CTX112 (based on CAR⁺ T cells) is administered as a single IV infusion. A dose limit of 7×10⁴ TCR⁺ cells/kg per CTX112 infusion is imposed for all dose levels. The total dose may be contained in 1 or multiple vials. Infusion must occur through a central venous catheter. A leukocyte filter must not be used.

Prior to the start of CTX112 infusion, the site pharmacy must ensure that 2 doses of tocilizumab and emergency equipment are available for each specific subject treated. Subjects should be premedicated per the site standard of practice with acetaminophen orally (PO) (i.e., paracetamol or its equivalent per site formulary) and diphenhydramine hydrochloride IV or PO (or another H1-antihistamine per site formulary) approximately 30 to 60 minutes prior to CTX112 infusion.

Prophylactic systemic corticosteroids should not be administered, as they may interfere with the activity of CTX112.

CTX112 infusion is delayed if any of the following signs or symptoms are present:
New active uncontrolled infection
Worsening of clinical status compared to prior to start of LD chemotherapy that places the subject at increased risk of toxicity
Neurotoxicity known to increase risk of ICANS (e.g., seizures, stroke, change in mental status). Neurotoxicity of benign origin (e.g., headache) lasting less than 48 hours and considered reversible is allowed.

CTX112 infusion is administered at least 48 hours (but no more than 7 days) after the completion of LD chemotherapy. If CTX112 infusion is delayed by more than 10 days, LD chemotherapy must be repeated.

CTX112 Postinfusion Monitoring

Following CTX112 infusion, subject's vitals should be monitored every 30 minutes for 2 hours after infusion or until resolution of any potential clinical symptoms.

During Phase 1, all subjects are hospitalized for observation for the first 7 days after CTX112 infusion. For all phases of the study, subjects must remain in proximity of the investigative site (i.e., 1-hour transit time) for at least 28 days after CTX112 infusion, and postinfusion hospitalization may be required or extended by local regulation or site practice.

During this acute toxicity monitoring period, subjects are routinely assessed for AEs, including CRS, TLS, ICANS, and GvHD according to the schedule of assessments (Tables 22 and 23).

Subjects should remain hospitalized until CTX112-related nonhematologic toxicities (e.g., fever, hypotension, hypoxia, ongoing neurological toxicity) return to grade 1 or resolve.

Administration of an Additional CTX112 Infusion after Disease Progression

Initially, the dose regimen consists of LD chemotherapy followed by a single CTX112 infusion. However, subjects who achieve an initial response to CTX112 (PR or better) and subsequently experience disease progression may be eligible for an additional CTX112 infusion. The dose would be at or below the highest dose level (i.e., a dose level enrolling in Phase 1 Part A or, if applicable, a dose level enrolling for subjects with the same disease histology in the respective Phase 1 Part B disease-specific cohort).

All subjects must meet safety and eligibility criteria described in the protocol before receiving any additional CTX112 infusions.

Subjects who achieve PR or better have the option to receive a second course of treatment with lymphodepleting chemotherapy and an additional CTX112 infusion if they meet the following criteria:
Confirmation tumor remains CD19⁺ at relapse (by flow cytometry or IHC)
No prior DLT during dose escalation (if applicable)
No prior grade ≥3 CRS without resolution to grade ≤2 within 72 hours following CTX112 infusion
No prior GvHD following CTX112 infusion
No prior grade 4 ICANS following CTX112 infusion and any grade 1, 2, or 3 ICANS must have resolved more than 14 days prior to a subsequent CTX112 infusion
Meet initial study inclusion criteria (#1, #2, #5 through #9) and exclusion criteria (#2 [except prior treatment with CTX112] through #15)
Meet criteria for LD chemotherapy and CTX112 infusion

All screening assessments (see schedule of assessments in Table 22) must be repeated, including the following additional considerations:
Brain MRI and lumbar puncture (LP) to be repeated if at high risk for CNS recurrence (e.g., subjects with high-grade B cell lymphoma with MYC and BCL2 and/or BCL6 rearrangement or double expressor lymphoma; subjects with testicular involvement of lymphoma; subjects with DLBCL treated with R-CHOP with high-risk scores on the CNS International Prognostic Index (IPI) criteria (Schmitz et al., 2016, J Clin Oncol 34, 3150-3156); or subjects with other disease histologies assessed as high risk for CNS involvement) at the time of the additional infusion. LP may be omitted if the additional infusion occurs within 3 months of last CTX112 infusion when the LP was performed and if no new CNS symptoms are present.
Echocardiogram may be omitted if the additional infusion occurs within 3 months of last CTX112 infusion when echocardiogram was performed and if no new cardiac symptoms have occurred.
The PET/CT scan demonstrating disease relapse/progression serves as the new baseline for tumor response evaluation. The additional infusion must occur within 28 days of that scan. Bone marrow aspirate and biopsy must be repeated if it was not performed at the time of relapse/progression.

A maximum of 1 additional infusion may occur per subject. Subjects who receive an additional CTX112 infusion follow the same treatment schedule and procedural requirements per the first course of treatment (i.e., the schedule of assessments, safety reporting periods, and long-term follow up restart at the time an additional CTX112 infusion is administered).

For subjects who receive an additional CTX112 infusion after PD, disease response is assessed relative to the most recent PET/CT scan and bone marrow prior to the additional infusion.

Prior and Concomitant Medications

Allowed Medications

Necessary supportive measures for optimal medical care is given throughout the study, including IV antibiotics to treat infections, growth factors, blood components, etc., except for prohibited medications listed below. Steroids administered for the purposes of disease control must be stopped within 14 days or 5 half-lives, whichever is longer, prior to enrollment.

All concurrent therapies, including prescription and nonprescription medication, must be recorded from the date of signed informed consent through 3 months after CTX112 infusion. Beginning 3 months post-CTX112 infusion, only the following selected concomitant medications are collected: IV Igs, vaccinations, anticancer treatments (e.g., chemotherapy, radiation, immunotherapy), immunosuppressants (including steroids), and any investigational agents.

Prohibited Medications

The following medications are prohibited during certain periods of the study as specified below:
Corticosteroid therapy at a pharmacologic dose (>10 mg/day of prednisone or equivalent doses of other corticosteroids) and other immunosuppressive drugs should be avoided after CTX112 administration unless medically indicated to treat new toxicity or as part of management of CRS or ICANS associated with CTX112.
Granulocyte-macrophage colony-stimulating factor following CTX112 infusion due to the potential to worsen symptoms of CRS.
Granulocyte colony-stimulating factor (G-CSF) is prohibited following CTX112 infusion but may be administered ≥10 days following CTX112 infusion if subject shows no signs of CRS. After at least 3 subjects have been dosed with CTX112, earlier use of G-CSF may be allowed, if deemed appropriate based on emerging safety data.
Intrathecal CNS prophylaxis must be stopped at least 7 days prior to CTX112 infusion.
Any anticancer therapy (e.g., chemotherapy, immunotherapy, targeted therapy, radiation or other investigational agents) other than LD chemotherapy prior to disease progression

### 6. Toxicity Management

General Guidance

Subjects must be closely monitored for at least 28 days after CTX112 infusion. General recommendations are provided below:
Fever is the most common early manifestation of CRS; however, subjects may also experience weakness, hypotension, or confusion as first presentation.
Diagnosis and management of CRS should be based on clinical symptoms and NOT laboratory values.
In subjects who do not respond to CRS-specific management, always consider sepsis and resistant infections. Subjects should be continually evaluated for resistant or emergent bacterial infections, as well as fungal or viral infections.
CRS, including HLH, and TLS may occur at the same time following CAR T cell infusion. Subjects should be consistently monitored for signs and symptoms of all the conditions and managed appropriately.
Neurotoxicity may occur at the time of CRS, during CRS resolution, or following resolution of CRS. Grading and management of neurotoxicity will be performed separately from CRS.
Tocilizumab must be administered within 2 hours from the time of order.

Toxicity-Specific Guidance

Infusion Reactions

Infusion reactions have been reported in autologous CD19-directed CAR T cell studies, including transient fever, chills, and/or nausea. Acetaminophen (paracetamol) and diphenhydramine hydrochloride (or another H1-antihistamine) may be repeated every 6 hours after CTX112 infusion, as needed, if an infusion reaction occurs. For subjects who do not respond to initial treatment, additional therapy (e.g., H2 antagonist, nonsteroidal anti-inflammatory medication) may be considered per institutional practices. Systemic corticosteroids should be avoided, as they may interfere with the activity of CTX112.

Febrile Reaction and Infection Prophylaxis

Infection prophylaxis should occur according to the institutional standard of care.

In the event of febrile reaction, an evaluation for infection should be initiated and the subject managed appropriately with antibiotics, fluids, and other supportive care as medically indicated and determined by the treating physician. Viral and fungal infections should be considered throughout a subject's medical management if fever persists. If a subject develops sepsis or systemic bacteremia following CTX112 infusion, appropriate cultures and medical management should be initiated. Additionally, consideration of CRS should be given in any instances of fever following CTX112 infusion within 30 days postinfusion.

For subjects receiving a CTX112 infusion with LD chemotherapy after initial clinical response and subsequent progressed disease, Pneumocystis jirovecii prophylaxis is recommended per local guidelines.

Tumor Lysis Syndrome

Subjects receiving CAR T cell therapy are at increased risk of TLS. Subjects should be closely monitored for TLS via laboratory assessments and symptoms from the start of LD chemotherapy until 28 days following CTX112 infusion.

All subjects at risk for TLS should receive prophylactic allopurinol (or a nonallopurinol alternative, such as febuxostat, per institutional guidelines) and increased oral/IV hydration during screening and before initiation of LD chemotherapy. Prophylaxis can be stopped after 28 days following CTX112 infusion or once the risk of TLS passes.

Sites should monitor and treat TLS as per their institutional standard of care, or according to published guidelines (Cairo and Bishop, 2004). TLS management, including administration of rasburicase, should be instituted promptly when clinically indicated.

Cytokine Release Syndrome

Cytokine release syndrome (CRS) is a major toxicity reported with autologous CD19-directed CAR T cell therapy. CRS is due to hyperactivation of the immune system in response to CAR engagement of the target antigen, resulting in multi-cytokine elevation from rapid T cell stimulation and proliferation (Frey et al., 2014, Blood 124, 2296; Maude et al., 2014, Cancer J 20, 119-122). When cytokines are released, a variety of clinical signs and symptoms associated with CRS may occur, including cardiac, gastrointestinal (GI), neurological, respiratory (dyspnea, hypoxia), skin, cardiovascular (hypotension, tachycardia), and constitutional (fever, rigors, sweating, anorexia, headaches, malaise, fatigue, arthralgia, nausea, and vomiting) symptoms, and laboratory (coagulation, renal, and hepatic) abnormalities.

The goal of CRS management is to prevent life-threatening sequelae while preserving the potential for the antitumor effects of CTX112. Symptoms usually occur 1 to 14 days after autologous CAR T cell therapy, but the timing of symptom onset has not been fully defined for allogeneic CAR T cells.

Cytokine release syndrome should be identified and treated based on clinical presentation and not laboratory cytokine measurements. If CRS is suspected, grading should be applied according to the ASTCT (formerly known as American Society for Blood and Marrow Transplantation) consensus recommendations (Lee et al., 2019) (Table 14), and management should be performed according to the recommendations in Table 15.

Neurotoxicity is graded and managed as described herein.

TABLE 14
Grading of CRS According to ASTCT Consensus Criteria

| CRS Parameter | Grade 1 | Grade 2 | Grade 3 | Grade 4 |
|---|---|---|---|---|
| Fever1 | Temperature ≥38° C. | Temperature ≥38° C. | Temperature ≥38° C. | Temperature ≥38° C. |
| With | None | Not requiring | Requiring a | Requiring multiple |
| hypotension | | vasopressors | vasopressor with or | vasopressors |
| | | | without vasopressin | (excluding |
| | | | | vasopressin) |
| And/or | None | Requiring low-flow | Requiring high- | Requiring positive |
| hypoxia2 | | nasal cannula³ or | flow nasal cannula³, | pressure (e.g., |
| | | blow-by | facemask, | CPAP, BiPAP, |
| | | | nonrebreather | intubation, and |
| | | | mask, or Venturi | mechanical |
| | | | mask | ventilation) |

ASTCT: American Society for Transplantation and Cellular Therapy; BiPAP: bilevel positive airway pressure; C: Celsius; CPAP: continuous positive airway pressure; CRS: cytokine release syndrome; CTCAE: Common Terminology Criteria for Adverse Events.
Note:
Organ toxicities associated with CRS may be graded according to CTCAE v5.0 but they do not influence CRS grading.
¹Fever is defined as temperature ≥38° C. not attributable to any other cause. In subjects who have CRS then receive antipyretics or anti-cytokine therapy such as tocilizumab or steroids, fever is no longer required to grade subsequent CRS severity. In this case, CRS grading is driven by hypotension and/or hypoxia.
²CRS grade is determined by the more severe event: hypotension or hypoxia not attributable to any other cause. For example, a subject with temperature of 39.5° C., hypotension requiring 1 vasopressor, and hypoxia requiring low-flow nasal cannula is classified as grade 3 CRS.
³Low-flow nasal cannula is defined as oxygen delivered at ≤6 L/minute. Low flow also includes blow-by oxygen delivery, sometimes used in pediatrics. High-flow nasal cannula is defined as oxygen delivered at >6 L/minute.

TABLE 15
CRS Management Guidance

| CRS | | |
|---|---|---|
| Severity¹ | Tocilizumab | Corticosteroids |
| Grade 1 | If <72 hours post infusion OR if | If <72 hours post infusion OR if symptoms (e.g., |
| | symptoms (e.g., fever) not improving | fever) not improving after 24 hours, consider |
| | after 24 hours, consider managing as | managing as Grade 2. |
| | Grade 2. | |
| Grade 2 | Administer tocilizumab² 8 mg/kg IV | Administer dexamethasone (e.g., 10 mg IV once |
| | over 1 hour (not to exceed 800 mg) | daily or 1 to 3 days) |
| | If improving, discontinue | If improving, manage as Grade 1 above |
| | tocilizumab. | and continue corticosteroids until the severity |
| | If no clinical improvement in | is Grade 1 or less, then quickly taper as |
| | the signs and symptoms of CRS | clinically appropriate. |
| | after the first dose, repeat | If not improving, manage as appropriate |
| | tocilizumab as early as 4 hours | grade below. |
| | later. Limit to a maximum of 3 | |
| | doses in a 24-hour period; | |
| | maximum total of 4 doses. | |
| | If still not improving, consider | |
| | siltuximab (e.g., 11 mg/kg IV once | |
| | per day) or anakinra (e.g., 100 mg | |
| | subcutaneously 1 to 3 times per | |
| | day). | |
| Grade 3 | Per grade 2 | Administer dexamethasone (e.g., 20 mg IV every |
| | If improving, manage as | 6-12 hours for 1 to 3 days). |
| | appropriate grade above. | If improving, manage as appropriate grade |
| | | above and continue corticosteroids until the |
| | | severity is Grade 1 or less, then quickly taper |
| | | as clinically appropriate. |
| | | If not improving, manage as Grade 4. |
| Grade 4 | Per grade 2 | Administer dexamethasone 20 mg IV every 6-12 |
| | If improving, manage as | hours for 1 to 3 days then taper as clinically |
| | appropriate grade above. | appropriate If improving, manage as appropriate grade |
| | | above and continue corticosteroids until the |
| | | severity is Grade 1 or less, then taper as |
| | | clinically appropriate. |
| | | If not improving, or for life threatening |
| | | CRS, consider methylprednisolone 1000 mg 1 |
| | | to 3 times per day or alternate therapy (which |
| | | may include, but is not limited to, anakinra, |
| | | siltuximab, ruxolitinib, cyclophosphamide, IV |
| | | immunoglobulin, or antithymocyte globulin). |

CRS: cytokine release syndrome; IV: intravenously; N/A: not applicable.
¹See (Lee et al., 2019)
²Refer to tocilizumab prescribing information.

Throughout the duration of CRS, subjects should be provided with supportive care consisting of antipyretics, IV fluids, and oxygen. Subjects who experience grade ≥2 CRS (e.g., hypotension, or hypoxia requiring supplemental oxygenation) should be monitored with continuous cardiac telemetry and pulse oximetry. For subjects experiencing grade 3 CRS, consider performing an echocardiogram to assess cardiac function. For grade 3 or 4 CRS, consider intensive care supportive therapy. Intubation for airway protection due to neurotoxicity (e.g., seizure) and not due to hypoxia should not be captured as grade 4 CRS. Similarly, prolonged intubation due to neurotoxicity without other signs of CRS (e.g., hypoxia) is not considered grade 4 CRS. The potential of an underlying infection should always be considered in cases of severe CRS, as the presentation (fever, hypotension, hypoxia) is similar. Resolution of CRS is defined as resolution of fever (temperature ≥38° C.), hypoxia, and hypotension (Lee et al., 2019)

Neurotoxicity

Neurotoxicity has been observed with autologous CD19-directed CAR T cell therapies. Signs and symptoms can be progressive and may include headache, aphasia, altered level of consciousness, impairment of cognitive skills, motor weakness, seizures, and cerebral edema. Neurotoxicity may occur at the time of CRS, during the resolution of CRS, or following resolution of CRS, and its pathophysiology is unclear. The ASTCT consensus further defined neurotoxicity associated with CRS as ICANS, a disorder characterized by a pathologic process involving the CNS following any immune therapy that results in activation or engagement of endogenous or infused T cells and/or other immune effector cells (Lee et al., 2019). Potential etiologies of neurotoxicity include ICANS or viral infection.

Evaluation of any new onset neurotoxicity should include a neurological examination, including ICE assessment tool (Table 17), brain MRI, and examination of the CSF (via LP), as clinically indicated. If a brain MRI is not possible, all subjects should receive a noncontrast CT to rule out intracerebral hemorrhage. LP is required for any grade ≥3 neurotoxicity and is strongly recommended for grade 1 and grade 2 events, if clinically feasible. LP must be performed within 48 hours of symptom onset, unless not clinically feasible. Electroencephalogram should also be considered as clinically indicated. Endotracheal intubation may be needed for airway protection in severe cases.

Nonsedating, antiseizure prophylaxis (e.g., levetiracetam) should be considered in all subjects for at least 21 days following CTX112 infusion or upon resolution of neurological symptoms (unless the antiseizure medication is considered to be contributing to the detrimental symptoms). For severe or life-threatening neurologic toxicities, intensive care supportive therapy should be provided. Neurology consultation should always be considered. Monitor platelets and for signs of coagulopathy and transfuse blood products appropriately to diminish risk of intracerebral hemorrhage. For subjects who receive active steroid management for more than 3 days, antifungal and antiviral prophylaxis is recommended to mitigate a risk of severe infection with prolonged steroid use. Consideration for antimicrobial prophylaxis should also be given.

In cases of suspected ICANS, subjects should be graded according to the ICANS grading table (Table 16) which incorporates assessment of level of consciousness, presence/absence of seizures, motor findings, presence/absence of cerebral edema, and overall assessment of neurologic domains by using a modified assessment tool called the ICE (immune effector cell-associated encephalopathy) assessment tool (Table 17).

Headache, which may occur in a setting of fever or after chemotherapy, is very common but is a nonspecific symptom. Headache alone may not necessarily be a manifestation of ICANS and further evaluation should be performed. Weakness or balance problem resulting from deconditioning and muscle loss are excluded from definition of ICANS. Similarly, intracranial hemorrhage with or without associated edema may occur due to coagulopathies in these subjects and are also excluded from definition of ICANS. These and other neurotoxicities should be captured in accordance with CTCAE v5.0.

ICANS

If ICANS is diagnosed, management should be performed according to Table 18 depending on the ICANS grade (Table 16) and whether the subject has concurrent CRS or not. In addition, nonsteroidal agents (e.g., anakinra, etc.) may be considered for ICANS management after discussion with the CRISPR medical monitor (Neill et al., 2020, Pract Neurol 20, 285-293). Subjects who experience ICANS grade ≥2 should be monitored with continuous cardiac telemetry and pulse oximetry.

TABLE 16
ICANS Grading

| Neurotoxicity | | | | |
|---|---|---|---|---|
| Domain | Grade 1 | Grade 2 | Grade 3 | Grade 4 |
| ICE score¹ | 7-9 | 3-6 | 0-2 | 0 (subject is unarousable and |
| | | | | unable to undergo ICE |
| | | | | assessment) |
| Depressed | Awakens | Awakens | Awakens only to | Subject is unarousable or |
| level of | spontaneously | to voice | tactile stimulus | requires vigorous or repetitive |
| consciousness² | | | | tactile stimuli to arise; stupor |
| | | | | or coma |
| Seizure | N/A | N/A | Any clinical seizure, | Life-threatening prolonged |
| | | | focal or generalized, | seizure (>5 min) or repetitive |
| | | | that resolves rapidly, or | clinical or electrical seizures |
| | | | nonconvulsive seizures | without return to baseline in |
| | | | on EEG that resolve | between |
| | | | with intervention | |
| Motor | N/A | N/A | N/A | Deep focal motor weakness |
| findings³ | | | | such as hemiparesis or |
| | | | | paraparesis |
| Elevated ICP/ | N/A | N/A | Focal/local edema | Diffuse cerebral edema on |
| cerebral | | | on neuroimaging 4 | neuroimaging, decerebrate or |
| edema | | | | decorticate posturing, cranial |
| | | | | nerve VI palsy, papilledema, |
| | | | | or Cushing's triad |

CTCAE: Common Terminology Criteria for Adverse Events; EEG: electroencephalogram; ICANS: immune effector cell-associated neurotoxicity syndrome; ICE: immune effector cell-associated encephalopathy (assessment tool); ICP: intracranial pressure; N/A: not applicable.
ICANS grade is determined by the most severe event (ICE score, level of consciousness, seizure, motor findings, raised ICP/cerebral edema) not attributable to any other cause.
¹A subject with an ICE score of 0 may be classified as grade 3 ICANS if awake with global aphasia, but a subject with an ICE score of 0 may be classified as grade 4 ICANS if unarousable.
²Depressed level of consciousness should be attributable to no other cause (e.g., sedating medication).
³Tremors and myoclonus associated with immune effector therapies should be graded according to CTCAE v5.0 but do not influence ICANS grading.

TABLE 17
ICE Assessment

| | | Maximum | |
|---|---|---|---|
| Domain | Assessment | Score | |
| Orientation | Orientation to year, month, city, hospital | 4 | points |
| Naming | Name 3 objects (e.g., point to clock, pen, | 3 | points |
| | button) | | |
| Following | Ability to follow commands (e.g., “Show me | 1 | point |
| command | 2 fingers” or “Close your eyes and stick out | | |
| | your tongue”) | | |
| Writing | Ability to write a standard sentence (includes | 1 | point |
| | a noun and verb) | | |
| Attention | Ability to count backward from 100 by 10 | 1 | point |

ICE score is reported as the total number of points (0-10) across all assessments.

TABLE 18
ICANS Management Guidance

| | Management¹ | |
|---|---|---|
| Severity | Concurrent CRS | No Concurrent CRS |
| Grade 1 | Consider tocilizumab per management for grade | Consider dexamethasone 10 mg IV once |
| | 1 CRS (Table 14) | daily for 1 to 3 days |
| | Consider dexamethasone 10 mg IV once daily for | |
| | 1 to 3 days, unless subject already on equivalent | |
| | dose for CRS | |
| Grade 2 | Administer tocilizumab per management for | Administer dexamethasone (e.g., 10-20 mg |
| | grade 2 CRS (Table 14) | IV every 6-12 hours for 1 to 3 days) |
| | Administer dexamethasone (e.g., 10-20 mg IV | If improving, continue |
| | every 6-12 hours for 1 to 3 days), unless subject | corticosteroids until the severity is |
| | already on equivalent dose for CRS | Grade 1 or less, then quickly taper as |
| | If improving, continue corticosteroids | clinically appropriate. |
| | until the severity is Grade 1 or less, then | If not improving, consider adding |
| | quickly taper as clinically appropriate. | siltuximab (e.g., 11 mg/kg IV once |
| | If not improving, consider adding | per day or anakinra (e.g., 100 mg SC |
| | siltuximab (e.g., 11 mg/kg IV once per day | 1 to 3 times per day). Siltuximab and |
| | or anakinra (e.g., 100 mg SC 1 to 3 times | anakinra treatment should be |
| | per day). Siltuximab and anakinra treatment | discussed with the medical monitor |
| | should be discussed with the medical | |
| | monitor. | |
| Grade 3 | Same as grade 2 | Same as grade 2 |
| | If not improving, manage as Grade 4. | If not improving, manage as Grade 4. |
| Grade 4 | Administer tocilizumab per management for | Administer methylprednisolone 1000 mg |
| | grade 2 CRS (Table 14) | IV twice per day. |
| | Administer methylprednisolone 1000 mg IV | If improving, manage as |
| | twice per day unless subject already on | appropriate grade above and continue |
| | equivalent dose for CRS | corticosteroids until the severity is |
| | If improving, manage as appropriate | Grade 1 or less, then taper as |
| | grade above and continue corticosteroids | clinically appropriate. |
| | until the severity is Grade 1 or less, then | If not improving, consider 1000 |
| | taper as clinically appropriate. | mg of methylprednisolone |
| | If not improving, consider 1000 mg of | intravenously 3 to 4 times a day or |
| | methylprednisolone intravenously 3 to 4 | alternate therapy (which may include, |
| | times a day or alternate therapy (which may | but is not limited to, anakinra, |
| | include, but is not limited to, anakinra, | siltuximab, ruxolitinib, |
| | siltuximab, ruxolitinib, cyclophosphamide, | cyclophosphamide, intravenous |
| | intravenous immunoglobulin, or | immunoglobulin, or antithymocyte |
| | antithymocyte globulin) | globulin) |

CRS: cytokine release syndrome;
ICANS: immune effector cell-associated neurotoxicity syndrome;
IV: intravenously;
SC: subcutaneously
¹Consider levetiracetam for seizure prophylaxis for grade 1 ICANS. For grade ≥ 2 ICANS, start seizure prophylaxis/treatment if not already active

Viral Encephalitis

Reactivation of chronic latent infections has been reported in patients who are highly immunosuppressed, including those who have undergone hematopoietic stem cell transplantation (HSCT) or CAR T cell therapy (Handley et al., 2021, J Clin Virol 136, 104740; Rebechi et al., 2021, Open Forum Infect Dis 8, ofab470). Specifically, there have been 8 reported cases of human herpesvirus 6 (HHV-6) encephalitis following CAR T cell infusion (Spanjaart et al., 2022, N Engl J Med 386, 80-87).

Viral encephalitis can be distinguished from ICANS based on the characteristics described in Table 19. In addition, encephalitis should be considered in subjects who initially respond to ICANS treatment, but subsequently worsen.

TABLE 19
Differentiating Between ICANS and HHV-6 Encephalitis After CAR T Cell Therapy

| | Neurotoxicity After CAR T Cell | |
|---|---|---|
| | Therapy (ICANS) | HHV-6 Encephalitis |
| Clinical | Tremor, dysphagia, expressive aphasia, | Headache, confusion, or delirium; |
| presentation | impaired attention, apraxia, and lethargy | progresses to anterograde then |
| | | retrograde amnesia |
| Timing | Very early (usually within first | Typically within the first 21 days |
| | 3 to 10 days) | |
| Progression | Rapidly over hours to days | More slowly, over several days |
| Associated | Cytokine release syndrome | Fever is uncommon |
| symptoms | | |
| Seizures | Less common | Very common |
| CSF findings | Often normal, may have mildly | Mildly abnormal (minimal |
| | elevated protein | pleocytosis, slightly elevated |
| | | protein, normal glucose) |
| CSF HHV-6 | PCR negative | PCR positive |
| EEG abnormalities | Diffuse slowing | Epileptiform activity |
| Imaging findings | Usually normal, although vasogenic | T2/FLAIR/DWI hyperintensities in |
| (MRI) | edema, leptomeningeal enhancement, and | the medial temporal lobes, especially |
| | microhemorrhages | the amygdala and the hippocampus |
| Treatment | Corticosteroids and/or anti-IL-6 | Antivirals (ganciclovir, foscarnet, |
| | monoclonal antibodies | cidofovir) |
| Outcomes | Highly variable, ranges from complete | Highly variable, ranges from complete |
| | recovery to significant residual disability | recovery to disability and death; |
| | and death | majority recover |

CAR: chimeric antigen receptor; CSF: cerebrospinal fluid; EEG: electroencephalogram; HHV-6, human herpesvirus 6; IL-6: interleukin-6; MRI: magnetic resonance imaging; PCR: polymerase chain reaction; T2/FLAIR/DWI: T2-weighted fluid attenuated inversion recovery/diffusion-weighted imaging.
Source: Adapted from (Rebechi et al., 2021).

In cases of suspected viral encephalitis, the following diagnostic tests are required:
brain MRI,
blood (plasma preferred) for HHV-6 DNA PCR, and
LP for:
   HHV-6 DNA PCR (should be performed within 48 hours of symptoms if clinically feasible),
   the standard panel performed at site (which should include at least cell count, Gram stain, and Neisseria meningitidis), and
   CSF PCR analysis for herpes simplex virus (HSV)-1 and -2, enterovirus, varicella zoster virus (VZV), cytomegalovirus (CMV), and HHV-6. Baseline serology samples are collected and sent to a central lab and may be tested in the event of viral infection to distinguish between primary infection and reactivation.

Results from the infectious disease panel should be available within 5 business days of the LP in order to appropriately manage the subject.

HHV-6 management should include the following:
Primary therapy should include foscarnet and ganciclovir, and second-line therapy should include cidofovir.
Recommended duration of therapy is 3 weeks. Treatment duration can be increased to 6 weeks in the event of persistent clinical manifestations or ongoing detectable HHV-6 copies in the CSF or peripheral blood.
An infectious disease consultation is recommended.

B Cell Aplasia

B cell aplasia may occur and is monitored by following IgG blood levels. IV gammaglobulin is recommended for clinically significant hypogammaglobulinemia (systemic infections, <400 mg/dL) or according to institutional standard of care.

Hemophagocytic Lymphohistiocytosis

HLH has been reported after treatment with autologous CD19-directed CAR T cells (Barrett et al., 2014, Curr Opin Pediatr 26, 43-49; Maude et al., 2014, Cancer J 20, 119-122; Maude et al., 2015, Blood 125, 4017-4023; Porter et al., 2015, Sci Transl Med 7, 303ra139; Teachey et al., 2013, Blood 121, 5154-5157). HLH is a clinical syndrome that is a result of an inflammatory response following infusion of CAR T cells in which cytokine production from activated T cells leads to excessive macrophage activation. Signs and symptoms of HLH may include fevers, cytopenias, hepatosplenomegaly, hepatic dysfunction with hyperbilirubinemia, coagulopathy with significantly decreased fibrinogen, and marked elevations in ferritin and C-reactive protein (CRP). Neurologic findings have also been observed (Jordan et al., 2011, Blood 118, 4041-4052; La Rosée, 2015, Hematology 2015, 190-196).

CRS and HLH are overlapping clinical syndromes and have similar pathophysiology. HLH, if present, will likely occur at the time of CRS or as CRS is resolving. HLH has been considered an overlap syndrome with CRS and should be treated according to the CRS management guidelines.

The diagnosis of HLH is confirmed in subjects who meet the following criteria (Neelapu et al., 2020, J Clin Oncol 38, 8002): Peak serum ferritin level of >10,000 ng/mL during the CRS phase of CAR T cell therapy (typically the first 5 days after cell infusion, AND who subsequently develop any of the following:
Grade ≥3 increase in serum bilirubin, AST, or ALT levels
Grade ≥3 oliguria or increase in serum creatinine levels
Grade ≥3 pulmonary edema
Presence of hemophagocytosis in bone marrow or organs based on histopathological assessment of cell morphology and/or CD68 ICH

HLH may be suspected in the presence of unexplained elevated liver function tests or cytopenias with or without other evidence of CRS. In the event of a suspicion or diagnosis of HLH, the following should be collected:
Serum soluble interleukin-2 (soluble CD25)
Bone marrow biopsy, aspirate may be collected for further confirmation if safe to conduct. Where feasible, excess bone marrow samples should be sent to a central laboratory.

If HLH is suspected or confirmed:
Frequently monitor coagulation parameters, including fibrinogen. These tests may be done more frequently than indicated in the schedule of assessments, and frequency should be driven based on laboratory findings.
Fibrinogen should be maintained ≥100 mg/dL to decrease risk of bleeding.
Coagulopathy should be corrected with blood products.
Check for soluble CD25 and triglycerides.
If possible, perform bone marrow biopsy to assess for hemophagocytosis.
Given the overlap with CRS, subjects should be managed per CRS treatment guidance in Table 15. The IL-1 inhibitor anakinra or other anti-cytokine therapies (such as emapalumab-lzsg) may also be considered.

Cytopenias

Grade 3 neutropenia and thrombocytopenia, at times lasting more than 28 days postinfusion, have been reported in subjects treated with autologous CD19-directed CAR T cell products (KYMRIAH USPI, 2020; YESCARTA USPI, 2022). Therefore, subjects receiving CTX112 should be monitored for such toxicities and appropriately supported. Consideration should be given to antimicrobial and antifungal prophylaxis for any subject with prolonged neutropenia. For subjects experiencing grade ≥3 neutropenia, thrombocytopenia, or anemia that has not resolved within 28 days of CTX112 infusion, a complete blood count with differential should be performed twice a week until resolution to grade ≤2 or administration of a new systemic anticancer therapy weekly until Month 3 after each dose of CTX112, then a minimum of monthly until in accordance with institutional practice.

G-CSF may be considered in cases of grade 4 neutropenia 10 days post-CTX112 infusion, when the risk of CRS has passed. G-CSF administration may be considered earlier but must first be discussed with the medical monitor. Antimicrobial and antifungal prophylaxis should be considered for any subject with prolonged neutropenia or on high doses of steroids.

Graft vs Host Disease

Graft vs host disease (GvHD) is seen in the setting of allogeneic HSCT and is the result of immunocompetent donor T cells (the graft) recognizing the recipient (the host) as foreign. The subsequent immune response activates donor T cells to attack the recipient to eliminate foreign antigen-bearing cells. GvHD is divided into acute, chronic, and overlap syndromes based on both the time from allogeneic HSCT and clinical manifestations. Signs of acute GvHD may include a maculopapular rash; hyperbilirubinemia with jaundice due to damage to the small bile ducts, leading to cholestasis; nausea, vomiting, and anorexia; and watery or bloody diarrhea and cramping abdominal pain (Zeiser and Blazar, 2017).

Due to the specificity of CAR insertion at the TRAC locus, it is highly unlikely for a T cell to be both CAR⁺ and TCR⁺. Remaining TCR⁺ cells are removed during the manufacturing process by immunoaffinity chromatography on an anti-TCR antibody column to achieve a minimum threshold of TCR⁺ cells in the final product in order to ensure a limit of 7×10⁴ TCR⁺ cells/kg is imposed for all dose levels. This limit is lower than the limit of 1×10⁵ TCR⁺ cells/kg based on published reports on the number of allogeneic cells capable of causing severe GvHD during HSCT with haploidentical donors (Bertaina et al., 2014, Blood 124, 822-826). Through this specific editing, purification, and strict product release criteria, the risk of GvHD following CTX112 should be low, although the true incidence is unknown. Subjects should be monitored closely for signs of acute GvHD following infusion of CTX112. The timing of potential symptoms is unknown. However, given that CAR T cell expansion is antigen-driven and will likely occur only in TCR⁻ cells, it is unlikely that the number of TCR⁺ cells will appreciably increase above the number infused.

Diagnosis and grading of GvHD should be based on published criteria (Harris et al., 2016), as outlined in Table 20.

TABLE 20
Criteria for Grading Acute GvHD

| | Skin (active | Liver | | Lower GI |
|---|---|---|---|---|
| Stage | erythema only) | (bilirubin mg/dL) | Upper GI | (stool output/day) |
| 0 | No active | <2 | No or intermittent | <500 mL/day |
| | (erythematous) | | nausea, vomiting, | or <3 episodes/ |
| | GvHD rash | | or anorexia | day |
| 1 | Maculopapular | 2-3 | Persistent nausea, | 500-999 mL/day or |
| | rash <25% BSA | | vomiting, or | 3-4 episodes/day |
| | | | anorexia | |
| 2 | Maculopapular | 3.1-6 | — | 1000-1500 mL/day |
| | rash 25-50% BSA | | | or 5-7 episodes/day |
| 3 | Maculopapular | 6.1-15 | — | >1500 mL/day |
| | rash >50% BSA | | | or >7 episodes/day |
| 4 | Generalized | >15 | — | Severe abdominal pain |
| | erythroderma | | | with or without ileus, or |
| | (>50% BSA) plus | | | grossly bloody stool |
| | bullous formation and | | | (regardless of stool |
| | desquamation >5% BSA | | | volume) |

BSA: body surface area; GI: gastrointestinal; GvHD: graft vs host disease.

Overall GvHD grade is determined based on most severe target organ involvement.
Grade 0: No stage 1-4 of any organ
Grade 1: Stage 1-2 skin without liver, upper GI, or lower GI involvement
Grade 2: Stage 3 rash and/or stage 1 liver and/or stage 1 upper GI and/or stage 1 lower GI
Grade 3: Stage 2-3 liver and/or stage 2-3 lower GI, with stage 0-3 skin and/or stage 0-1 upper GI
Grade 4: Stage 4 skin, liver, or lower GI involvement, with stage 0-1 upper GI

Potential confounding factors that may mimic GvHD such as infections and reactions to medications should be ruled out. Skin and/or GI biopsy should be obtained for confirmation before or soon after treatment has been initiated. In instance of liver involvement, liver biopsy should be attempted if clinically feasible. Sample(s) of all biopsies will also be sent to a central laboratory for pathology assessment.

Recommendations for management of acute GvHD are outlined in Table 21. To allow for intersubject comparability at the end of the study, These recommendations should be followed except in specific clinical scenarios in which following them could put the subject at risk.

TABLE 21
Acute GvHD Management

| Grade | Management |
|---|---|
| 1 | Skin: Topical steroids or immunosuppressants; if stage 2: |
| | methylprednisolone 1 mg/kg (or equivalent dose). |
| 2-4 | Initiate methylprednisolone 2 mg/kg daily (or equivalent |
| | dose). |
| | IV form of steroid such as methylprednisolone should be |
| | considered if there are concerns with malabsorption. |
| | Steroid taper may begin after improvement is seen after ≥3 |
| | days of steroids. Taper should be 50% decrease of total |
| | daily steroid dose every 5 days. |
| | GI: In addition to steroids, start anti-diarrheal agents |
| | per standard practice. |

GI: gastrointestinal; IV: intravenous.

Decisions to initiate second-line therapy should be made sooner for subjects with more severe GvHD. For example, secondary therapy may be indicated after 3 days with progressive manifestations of GvHD, after 1 week with persistent grade 3 GvHD, or after 2 weeks with persistent grade 2 GvHD. Second-line systemic therapy may be indicated earlier in subjects who cannot tolerate high-dose glucocorticoid treatment (Martin et al., 2012, Biol Blood Marrow Transplant 18, 1150-1163). Choice of secondary therapy and when to initiate the same is based on clinical judgment and local practice (including ASTCT and/or European Society for Blood and Marrow Transplantation [EBMT] guidelines) Holler et al., 2019, In The EBMT Handbook: Hematopoietic Stem Cell Transplantation and Cellular Therapies [Internet], E. Carreras, C. Dufour, M. Mohty, and N. Krager, eds. (Cham (CH): Springer); Lee et al., 2019).

Management of refractory acute GvHD or chronic GvHD is per institutional guidelines. Anti-infective prophylaxis measures should be instituted per local guidelines when treating subjects with immunosuppressive agents (including steroids).

Hypotension and Renal Insufficiency

Hypotension and renal insufficiency have been reported with CAR T cell therapy and should be treated with IV administration of normal saline boluses according to institutional practice guidelines. Dialysis should be considered when appropriate.

Special Consideration During COVID-19 Pandemic

Subjects enrolled in this study undergo LD chemotherapy, are immunocompromised, and at increased risk of infections. Hence, the clinical study protocol requires exclusion of subjects in the case of any ongoing active infection during screening, prior to LD chemotherapy, and prior to CTX112 infusion, or delayed infusions.

This measure includes subjects with active infection with Severe Acute Respiratory Syndrome Coronavirus-2 (SARS-CoV-2), the causal agent of COVID-19 (coronavirus disease-2019). Due to the rapidly changing evidence as well as locoregional differences, local regulations and institutional guidelines are followed if the current situation allows a safe conduct of the study for an individual subject at a given time. Whenever possible, a COVID-19 vaccine or booster should not be administered 4 weeks prior to scheduled imaging for disease assessment (Minamimoto and Kiyomatsu, 2021). Additionally, the minimal requirements are to be defined in a memorandum to the study centers that is periodically updated as evidence and guidelines evolve.

### 7. Study Procedures

The study consists of 3 distinct stages: (1) screening and eligibility confirmation; (2) treatment, consisting of LD chemotherapy and CTX112 infusion; and (3) follow up. During the screening stage, subjects are assessed according to the eligibility criteria.

After enrollment, subjects receive LD chemotherapy followed by infusion of CTX112. During follow up, subjects are assessed for tumor response, disease progression, and survival. Throughout all study stages, subjects are regularly monitored for safety.

A complete schedule of assessments is provided in Tables 22 and 23. Descriptions of all required study procedures are provided in this section. In addition to protocol-mandated assessments, subjects should be followed per institutional guidelines, and unscheduled assessments should be performed when clinically indicated.

Missed evaluations should be rescheduled and performed as close to the original scheduled date as possible. An exception is made when rescheduling becomes medically unnecessary or unsafe because it is too close in time to the next scheduled evaluation. In that case, the missed evaluation should be abandoned.

For the purposes of this protocol, there is no Day 0. All visit dates and windows are to be calculated using Day 1 as the date of CTX112 infusion. Each monthly visit should occur at least 30 days after the previous visit.

TABLE 22
Schedule of Assessments (Screening to Month 24)

| | | Treatment | | | | | Follow Up | | | | | | | | | | | | | | | |
|---|---|---|---|---|---|---|---|---|---|---|---|---|---|---|---|---|---|---|---|---|---|---|
| Study | | | | | D | D | | D | D | D | D | D | D | D | M | M | M | M | M | M | M | M |
| Stage | Screen- | LDC | LDC | LDC | 1⁽²⁾ | 1⁽²⁾ | | 3 ± | 5 ± | 8 ± | 10 ± | 14 ± | 21 ± | 28 ± | 2 ± | 3 ± | 6 ± | 9 ± | 12 ± | 15 ± | 18 ± | 24 ± |
| Day | ing¹ | D −3 | D −2 | D −1 | (pre) | (post) | D 2 | 1 d | 2 d | 2 d | 2 d | 2 d | 2 d | 4 d | 7 d | 14 d | 14 d | 14 d | 14 d | 14 d | 14 d | 21 d |

| | | | | | | | | | | | | | | | | | | | | | |
|---|---|---|---|---|---|---|---|---|---|---|---|---|---|---|---|---|---|---|---|---|---|
| Informed | X | | | | | | | | | | | | | | | | | | | | |
| consent | | | | | | | | | | | | | | | | | | | | | |
| Confirm | X | X | | | X | | | | | | | | | | | | | | | | |
| eligibility³ | | | | | | | | | | | | | | | | | | | | | |
| Enrollment | X | | | | | | | | | | | | | | | | | | | | |
| Medical | X | | | | | | | | | | | | | | | | | | | | |
| history⁴ | | | | | | | | | | | | | | | | | | | | | |
| Physical | X | X | X | X | X | X | X | X | X | X | X | X | X | X | X | X | X | X | X | X | X |
| exam⁵ | | | | | | | | | | | | | | | | | | | | | |
| Vital | X | X | X | X | X | X | X | X | X | X | X | X | X | X | X | X | X | X | X | X | X |
| signs⁶ | | | | | | | | | | | | | | | | | | | | | |
| Height, | X | X | X | X | X | | | | X | | X | | X | X | X | X | X | X | X | X | X |
| weight⁷ | | | | | | | | | | | | | | | | | | | | | |
| Pregnancy | X | X | | | | | | | | | | | | | | | | | | | |
| test⁸ | | | | | | | | | | | | | | | | | | | | | |
| ECOG status | X | | | | X | | | | | | | | X | | X | | | | | | |
| Brain MRI | X | | | | | | | | | | | | | | | | | | | | |
| Lumbar | X | | | | | | | | | | | | | | | | | | | | |
| puncture⁹ | | | | | | | | | | | | | | | | | | | | | |
| Echocardiogram | X | | | | | | | | | | | | | | | | | | | | |
| 12-lead ECG | X | X¹⁰ | | | X | | | | | | | | X | | | | | | | | |
| ICE | X | | | | X | X | X | X | X | | | | X | | | | | | | | |
| assessment* | | | | | | | | | | | | | | | | | | | | | |
| Patient- | X | | | | X | | | | | | | | X | | X | X | X | X | | X | X |
| reported | | | | | | | | | | | | | | | | | | | | | |
| outcomes¹¹ | | | | | | | | | | | | | | | | | | | | | |

| Concomitant | Continuous | |
|---|---|---|
| meds¹² | | |
| Adverse | Continuous | |
| events¹³ | | |
| Hospital | | Continuous |
| utilization | | |

Treatment

| | | | |
|---|---|---|---|
| LDC¹⁴ | X | X | X |

| | |
|---|---|
| CTX112 | X |
| infusion¹⁵ | |

NHL Disease Response/Assessment
(Central and Local)

| | | | | | | | | | |
|---|---|---|---|---|---|---|---|---|---|
| PET scan/ | X | | X | X | X | X | X | X | X |
| CT with | | | | | | | | | |
| IV contrast¹⁶ | | | | | | | | | |
| BM aspirate/ | X | | X | | | | | | |
| biopsy¹⁷ | | | | | | | | | |
| Tumor | X¹⁹ | X¹⁸ | | | | | | | |
| biopsy/ | | | | | | | | | |
| pathology¹⁸ | | | | | | | | | |

CLL/SLL Disease Response/Assessment
(Central and Local)

| | | | | | | | | | |
|---|---|---|---|---|---|---|---|---|---|
| CLL/SLL | X | | | | | | | | |
| disease risk | | | | | | | | | |
| labs and | | | | | | | | | |
| disease | | | | | | | | | |
| staging²⁰ | | | | | | | | | |
| PE, CBC, and | | X | X | X | X | X | X | x | X |
| B symptoms | | | | | | | | | |
| for CLL/SLL | | | | | | | | | |
| response | | | | | | | | | |
| assessment²¹ | | | | | | | | | |
| CT with IV | X | X²² | X²² | X²² | X²² | X²² | X²² | X²² | X²² |
| contrast²² | | | | | | | | | |

| BM aspirate/ | X | X²⁴ | | |
|---|---|---|---|---|
| biopsy²³ | | (required at time of CR/CRi and | | |
| | | as clinically indicated) | | |
| MRD assessment | X | | X | X²⁴ |
| (blood and | | | | |
| BM)²⁴ | | | | |

| | | |
|---|---|---|
| Tumor biopsy/ | X | X (Additional biopsy is required only to confirm |
| pathology²⁵ | | suspected disease progression or more aggressive |
| | | histology [e.g., Richter's transformation]) |

Laboratory Assessments
(Local)

| | | | | | | | | | | | | | | | | | | | | | |
|---|---|---|---|---|---|---|---|---|---|---|---|---|---|---|---|---|---|---|---|---|---|
| CBC w/ | X | X | X | X | X | X | X | X | X | X | X | X | X* | X | X | X | X | X | X | X | X |
| differential | | | | | | | | | | | | | | | | | | | | | |
| Serum | X | X | X | X | X | X | X | X | X | X | X | X | X | X | X | X | X | X | X | X | X |
| chemistry | | | | | | | | | | | | | | | | | | | | | |
| Coagulation | X | X | | | X | X | X | X | X | X | X | | X | | | | | | | | |
| parameters | | | | | | | | | | | | | | | | | | | | | |
| Viral | X | | | | | | | | | | | | | | | | | | | | |
| serology²⁶ | | | | | | | | | | | | | | | | | | | | | |
| Immunoglobulins | X | | | | X | | | | | | | | X | X | X | X | X | X | X | X | x |
| Ferritin, | X | | | | X | X | X | X | X | X | X | X | X | | | | | | | | |
| CRP* | | | | | | | | | | | | | | | | | | | | | |
| Lymphocyte | X | X | | | X | X | X | X | X | X | X | X | X | X | X | X | X | X | X | X | X |
| subset | | | | | | | | | | | | | | | | | | | | | |
| panel²⁷ | | | | | | | | | | | | | | | | | | | | | |
| B cell (CD19, | X | | | | X | | | | | | | | X | X | X | X | X | X | X | X | x |
| CD20) | | | | | | | | | | | | | | | | | | | | | |

Biomarkers (Blood, Central)

| | | | | | | | | | | | | | | | | | | |
|---|---|---|---|---|---|---|---|---|---|---|---|---|---|---|---|---|---|---|
| CTX112 | X | | X | X | X | X | X | X | X | X | X | X | X | X | X | | X | X |
| PK^{28, 29, *} | | | | | | | | | | | | | | | | | | |
| Cytokines^{29, *} | | X¹⁰ | X | | X | X | X | X | X | X | X | X | X | | | | | |
| Anti-CTX112 | X | | | | | | | | | | | X | | X | | | X | X |
| Ab²⁹ | | | | | | | | | | | | | | | | | | |
| Exploratory | X | X¹⁰ | X | X | | | X | X | X | X | X | X | X | X | X | X | x | X |
| biomarkers^{29, 30, 31, *} | | | | | | | | | | | | | | | | | | |

Ab: antibody; AE: adverse event; AESI: adverse event of special interest; BM: bone marrow: CBC: complete blood count; CNS: central nervous system; CR: complete response; CRi: complete remission with incomplete hematologic recovery; CRP: C-reactive protein; CRS: cytokine release syndrome; CT: computed tomography; D or d: day; ECG: electrocardiogram; ECOG: Eastern Cooperative Oncology Group; HBV: hepatitis B virus; HCV: hepatitis C virus; HHV-6: human herpesvirus 6; HIV-1/-2: human immunodeficiency virus type 1 or 2; HLH: hemophagocytic lymphohistiocytosis; ICE: immune effector cell-associated encephalopathy; IPI: International Prognostic Index; IV: intravenously; LDC: lymphodepleting chemotherapy; LP: lumbar puncture; M: month (assume 30 days); MRI: magnetic resonance imaging; MRD: minimal residual disease; NHL: non-Hodgkin lymphoma; NK: natural killer; PCR: polymerase chain reaction; PET: positron emission tomography; PK: pharmacokinetic(s); PRO: patient-reported outcome; relapsed/refractory: relapsed or refractory; SAE: serious adverse event; TBNK: T-, B-, natural killer (cells).
Note:
Study visits cannot be combined, even if visit windows allow overlap (e.g., D3 ± 1 day could overlap with D2 or D5 ± 2 days, but separate visits are required for the D2, D3, and D5 visits.)
*If a subject experiences CRS, HLH, neurotoxicity, or prolonged cytopenia, additional assessments may be requested:
If CNS symptoms persist, ICE assessment should continue to be performed approximately every 2 days until symptom resolution to grade 1 or baseline.
For subjects experiencing grade ≥3 neutropenia, thrombocytopenia, or anemia that has not resolved within 28 days after CTX112 infusion, a CBC with differential must be performed weekly until resolution to grade ≤2 or administration of a new systemic anticancer therapy until Month 3 after each dose of CTX112, then a minimum of monthly or in accordance with institutional practice.
Additional blood samples for PK, cytokines, ferritin/CRP, and exploratory biomarkers should be drawn; however, on days when any of these samples are already scheduled for collection, an additional sample is not needed.
If clinically feasible, LPs are performed during neurotoxicity.
¹Screening assessments to be completed up to 14 days after signing informed consent form. Imaging and biopsies performed 28 days prior to enrollment as part of standard of care may be used to satisfy screening requirements. Subjects allowed 1-time rescreening within 3 months of initial consent.
²All baseline assessments on Day 1 are performed prior to CTX112 infusion. Additional collections of PK and exploratory biomarkers should also be collected 20 (±5) minutes postinfusion.
³Enrollment eligibility confirmed before enrollment. Eligibility for treatment based on clinical status confirmed before LDC -3 and D1.
⁴Includes complete surgical and cardiac history.
⁵For subjects with CLL/SLL, systems of clinical relevance and associated with clinical signs/symptoms must be assessed (e.g., lymph nodes, liver, and spleen) if response is suspected.
⁶Includes sitting blood pressure, heart rate, respiratory rate, pulse oximetry, and temperature.
⁷Height at screening only.
⁸For female subjects of childbearing potential. Serum pregnancy test at screening. Serum or urine pregnancy test within 72 hours before start of LDC.
⁹LP at screening for subjects with high risk for CNS involvement (e.g., high-grade B cell lymphoma with MYC and BCL2 and/or BCL6 rearrangements or double-expressor lymphoma, subjects with testicular involvement of lymphoma, or subjects with DLBCL treated with R-CHOP with high-risk CNS IPI scores, or subjects with other disease histologies assessed as high risk for CNS involvement). For LPs performed during neurotoxicity, samples are sent to central laboratory for CTX112 PK and exploratory biomarkers whenever possible.
¹⁰Prior to initiation of LDC.
¹¹Phase 2 only: PRO surveys should be administered before any visit-specific procedures are performed.
¹²All concomitant medications are collected ≤3 months post-CTX112 infusion, after which only select concomitant medications are collected.
¹³Collect all AEs from informed consent to 3 months after CTX112 infusion and collect only SAEs, AESIs, and nonserious AEs related to LDC, study procedure, or CTX112 from 3 months after CTX112 infusion through Month 24 visit. After Month 24 to Month 60 or after a subject starts a new anticancer therapy anytime ≥3 months after CTX112 infusion, only SAEs and AESIs related to LDC study procedure, or CTX112 are reported.
¹⁴Start LDC within 7 days after study enrollment. After completion of LDC, ensure washout period of ≥48 hours (but ≤7 days) before CTX112 infusion.
¹⁵CTX112 is administered 48 hours to 7 days after completion of LDC.
¹⁶For subjects with NHL: Baseline disease assessment (PET scan/CT with IV contrast) performed within 28 days prior to enrollment. MRI with contrast allowed if CT is clinically contraindicated, or as required by local regulation. Additional imaging at Month 2 allowed.
¹⁷Performed ±5 days of visit date. Additional BM biopsy and aspirate should be performed to confirm CR as part of disease evaluation and to evaluate CTX112 trafficking. BM biopsy and aspirate may also be performed at time of disease relapse. Samples from all BM biopsy and aspirate should be sent for CTX112 PK and exploratory biomarkers. If HLH is suspected, BM biopsy and aspirate to be performed.
¹⁸For subjects who have disease amenable to biopsy. The biopsy on Day 3 may be performed within +5 days of visit date; however, Day 3 is preferred. If relapse occurs on study, every attempt should be made to obtain biopsy of relapsed tumor and send to central laboratory. Tumor biopsy refers to tumor tissue other than BM.
¹⁹Subjects will undergo tumor biopsy during screening for histopathological diagnosis of NHL subtype. However, if a biopsy of relapsed/refractory disease was performed after completion of the most recent line of therapy and within 3 months prior to enrollment, and an archival tissue sample is of sufficient volume and quality, archival tissue may be used in lieu of a biopsy at screening.
²⁰For subjects with CLL/SLL: Local assessment. Includes collection of peripheral blood lymphocytes. Laboratory analyses are performed. BALL prognostic score and disease stage (Binet and Rai Staging System) are determined.
²¹For subjects with CLL/SLL: Local assessment. Perform CBC with differential if not already scheduled, examine lymph nodes, liver, and spleen, and assess for absence of B symptoms.
²²For subjects with CLL/SLL: CT (neck, chest, abdomen, and pelvis) at screening and at each disease response assessment if indicated based on baseline evaluation. In addition, imaging should be performed at time of CR or PR. CT of diagnostic quality performed as part of PET/CT is acceptable provided bidimensional nodal and liver/spleen measurements are possible. Scan performed 28 days prior to enrollment as part of standard of care may be used for screening.
²³For subjects with CLL/SLL: BM aspirate and biopsy should be performed at screening and to confirm CR as part of disease evaluation. BM biopsy and aspirate may also be performed to assess cytopenia of uncertain cause (e.g., BM toxicity, disease progression) For any BM aspirates collected, samples should be sent for CTX112 levels and/or other exploratory analyses, including for assessment of MRD in Phase 1 Part B and Phase 2.
²⁴For subjects with CLL/SLL in Phase 1 Part B and Phase 2 only: Peripheral blood and BM aspirate samples are collected for central laboratory MRD assessment at screening and Month 3. Additional MRD blood and BM aspirate samples should be collected 1) for subjects at time of CR/CRi and 2) for subjects with no evidence of CLL in peripheral blood or BM but who had a PR based on residual lymphadenopathy or splenomegaly at Months 6, 9, 12, 15, 18, and 24.
²⁵For subjects with disease amenable to biopsy (e.g., SLL). Tumor biopsy refers to tumor tissue other than BM.
²⁶Infectious disease testing (HIV-1, HIV-2, HCV antibody/PCR, HBV surface antigen, HBV surface antibody, HBV core antibody) ≤45 days of enrollment may be considered for subject eligibility.
²⁷Lymphocyte subset panel assessment at screening, before start of first day of LDC, before CTX112 infusion, then all listed time points. To include 6-color TBNK panel, or equivalent for T, B, and NK cells (Table 30).
²⁸Samples for CTX112 PK should be sent to the central laboratory from any LP, BM aspirate, or tissue biopsy performed following CTX112 infusion.
²⁹Discontinuation of sample collection may be requested (e.g., if consecutive tests are negative). Continue sample collection for all listed time points.
³⁰Samples for exploratory biomarkers should be sent to the central laboratory from any LP or BM aspirate performed following CTX112 infusion.
³¹Whole blood is collected and sent to the central laboratory.

TABLE 23
Schedule of Assessments (Months 30-60)

| | M 30 (± | M 36 (± | M 42 (± | M 48 (± | M 54 (± | M 60 (± | Progressive | Secondary |
|---|---|---|---|---|---|---|---|---|
| Assessments | 21 days) | 21 days) | 21 days) | 21 days) | 21 days) | 21 days) | Disease¹ | Follow Up² |
| Vital signs³ | X | X | X | X | X | X | X | X |
| Physical exam | X | X | X | X | X | X | X | X |
| Concomitant | X | X | X | X | X | X | X | X |
| medications⁴ | | | | | | | | |
| Survival status | X | X | X | X | X | X | X | X |
| Disease assessment⁵ | X | X | X | X | X | X | X | |
| Tumor biopsy⁵ | | | | | | | X | |
| CBC with | X | X | X | X | X | X | X | X |
| differential⁶ | | | | | | | | |
| Serum chemistry⁶ | X | X | X | X | X | X | X | X |
| Immunoglobulins^{6, 7} | X | X | X | X | X | X | X | |
| Lymphocyte | X | X | X | X | X | X | X | |
| subset profile^{6, 7} | | | | | | | | |
| CTX112 PK | | X | | X | | X | X | X |
| (central)^{7, 8} | | | | | | | | |
| Exploratory | | X | | X | | X | X | X |
| biomarkers | | | | | | | | |
| (central)^{7, 9} | | | | | | | | |
| Anti-CTX112 | | X | | X | | X | X | |
| Ab (blood, | | | | | | | | |
| central)⁷ | | | | | | | | |
| Patient-reported | X | X | X | X | X | X | X | |
| outcomes¹⁰ | | | | | | | | |
| Adverse events¹¹ | X | X | X | X | X | X | X | X |
| Subsequent | X | X | X | X | X | X | X | X |
| anticancer | | | | | | | | |
| therapy | | | | | | | | |

Ab: antibody; AESI: adverse event of special interest; ALL: acute lymphoblastic leukemia; BM: bone marrow; CBC: complete blood count; CT: computed tomography; GvHD: graft vs host disease; iwCLL: International Workshop on Chronic Lymphocytic Leukemia; LDC: lymphodepleting chemotherapy; M: month; NHL: non-Hodgkin lymphoma; PD: progressive disease; PET: positron emission tomography; PK: pharmacokinetic; PRO: patient-reported outcome; SAE: serious adverse event; TBNK: T-, B-, natural killer (cells).
¹If it is determined that a subject has PD at a scheduled study visit, the subject must complete all assessments of the scheduled visit, as well as any additional assessments listed for the progressive disease time point. If it is determined that a subject has progressive disease at an unscheduled visit, the subject should complete all assessments for the progressive disease time point at that unscheduled visit.
²Subjects with PD or who receive a different line of anticancer therapy discontinue the normal schedule of assessments and attend annual study visits. Subjects who partially withdraw consent undergo these procedures at minimum.
³Includes temperature, blood pressure, pulse rate, and respiratory rate.
⁴Only select concomitant medications are collected.
⁵For subjects with NHL, disease assessment consists of review of physical exam, CBC, clinical chemistry, and lactate dehydrogenase for subjects with NHL. Subjects with suspected malignancy undergo PET/CT imaging and/or a BM biopsy to confirm relapse. Every attempt should be made to obtain a biopsy of the relapsed tumor in subjects who progress and to send the sample to the central laboratory. For subjects with CLL/SLL, disease evaluations based on assessments in accordance with iwCLL guidelines including PE, CBC with differential, and CT and/or BM biopsy as clinically indicated.
⁶Assessed at local laboratory. To include 6-color TBNK panel, or equivalent for T, B, and NK cells (please see Table 30).
⁷Continue sample collection for all listed time points until otherwise instructed.
⁸Samples for CTX112 PK analysis should be sent to the central laboratory from any LP, BM biopsy, or tissue biopsy performed following CTX112 infusion.
⁹Samples for exploratory biomarkers should be sent to the central laboratory from any LP or BM aspirate performed.
¹⁰Phase 2 only.
¹¹SAEs, AESIs, and nonserious AEs related to LDC, study procedure, or CTX112 should be reported until last study visit. Only SAEs and AESIs related to LD chemotherapy, study procedure, or CTX112 are reported after Month 24 to Month 60 or if a subject starts new anticancer therapy anytime ≥3 months after CTX112 infusion.

Subject Screening, Enrollment, and Withdrawal

Subject Screening and Enrollment

A log is kept of all potential subjects reviewed and evaluated for study participation. The screening log captures limited information such as date of screening, date of enrollment, and reason why a subject failed screening. If at any time in the screening period a subject fails to meet the eligibility criteria, the subject is designated a screen failure.

The screening period begins on the date that the subject signs the informed consent form (ICF) and continues through confirmation of eligibility and enrollment into the study. Once informed consent has been obtained, the subject is screened to confirm study eligibility as outlined in the schedule of assessments (Table 22). Screening assessments are to be completed up to 14 days after initial enrollment.

After screening is completed, a subject is deemed enrolled in the study upon confirmation of eligibility and approval of enrollment. Subjects should start LD chemotherapy within 7 days after study enrollment.

Subjects are allowed a one-time rescreening, which may take place within 3 months of the initial consent. If rescreening occurs, screening assessments should be repeated as outlined in the schedule of assessments (Table 22) with the following considerations:
Echocardiogram (unless new cardiac signs or symptoms), brain MRI, and LP (unless new neurological symptoms concerning for progression) are not required.
PET/CT and bone marrow biopsy and aspirate must be repeated if >4 weeks from planned CTX112 infusion.
Tumor biopsy does not need to be repeated if the initial screening sample was collected ≤3 months prior to rescreening.

Assignment of Subjects to Treatment Cohorts

In Phase 1 Part A, subjects with B cell malignancies, including FL grade 1-3a, MCL, MZL, CLL/SLL, and DLBCL NOS, high-grade B cell lymphoma with MYC and BCL2 and/or BCL6 rearrangements, PMBCL (Phase 2 only), transformed FL, grade 3b FL, transformed MCL, or transformed MZL are enrolled and allocated. In Phase 1 Part B and Phase 2, subjects are assigned to separate cohorts based on disease histology and prior treatments.

Progressive Disease Procedures

Subjects who are determined to have PD while attending a scheduled study visit must complete all assessments of the scheduled visit, as well as any additional assessments listed for the PD time point. Subject who are determined to have PD at an unscheduled visit should complete all assessments for the PD time point at that unscheduled visit.

Subjects who have progressed and will no longer receive study-specified treatment should remain on study and enter the Secondary Follow-Up Phase as described in Table 23.

Secondary Follow-Up Procedures

Subjects with PD or who receive a different line of anticancer therapy discontinue the normal schedule of assessments and attend annual study visits. The assessments listed under secondary follow up are performed at these annual visits. Subjects who partially withdraw consent undergo these procedures at minimum.

Subjects who enter secondary follow up due to disease progression and start of new anticancer therapy should attend annual visits through Month 60 to collect safety information, as described in Table 23 under secondary follow-up visit.

Study Assessments

Refer to the schedule of assessments in Tables 22-23 for the timing of the required procedures.

Medical History

Demographic data is collected. Medical history, including a full history of the subject's disease, previous cancer treatments, and response to treatment from date of diagnosis is obtained. Cardiac, neurological, and surgical history are obtained.

For study entry, all subjects must fulfill all inclusion criteria described, and have none of the exclusion criteria described.

Physical Exam

Physical examination, including examination of major body systems, including general appearance, skin, neck, head, eyes, ears, nose, throat, heart, lungs, abdomen, lymph nodes, extremities, and nervous system, are performed at every study visit and the results documented. Changes noted from the exam performed at screening are recorded as an AE.

For subjects with CLL/SLL, systems of clinical relevance and associated with clinical signs/symptoms must be assessed (e.g., lymph nodes, liver, and spleen) if response to CTX112 is suspected.

Vital Signs, Including Height and Weight

Vital signs are recorded at every study visit and include sitting blood pressure, heart rate, respiratory rate, pulse oximetry, temperature, and height. Weight is obtained according to the schedule in Table 22, and height will only be obtained at screening.

Pregnancy Test

Female subjects of childbearing potential (i.e., women who are postmenarcheal with an intact uterus and at least 1 ovary who are less than 1 year postmenopausal) must have a serum pregnancy test performed at the time of screening, and a serum or urine pregnancy test within 72 hours of beginning LD chemotherapy.

ECOG Performance Status

Performance status is assessed at the screening, CTX112 infusion (Day 1), Day 28, and Month 3 visits. Performance status is assessed using the ECOG scale to determine the subject's general well-being and ability to perform activities of daily life.

TABLE 24
ECOG Performance Status

| Grade | ECOG Performance Status |
|---|---|
| 0 | Fully active, able to carry on all pre-disease performance |
| | without restriction |
| 1 | Restricted in physically strenuous activity but ambulatory |
| | and able to carry out work of a light or sedentary nature, |
| | e.g., light house work, office work |
| 2 | Ambulatory and capable of all self-care but unable to carry |
| | out any work activities; up and about more than 50% of waking |
| | hours |
| 3 | Capable of only limited self-care; confined to bed or chair |
| | more than 50% of waking hours |
| 4 | Completely disabled; cannot carry on any self-care; totally |
| | confined to bed or chair |
| 5 | Dead |

Developed by the Eastern Cooperative Oncology Group, Robert L. Comis, MD, Group Chair (Oken et al., 1982, Am J Clin Oncol 5, 649-655).

Echocardiogram

A transthoracic cardiac echocardiogram (for assessment of left ventricular ejection fraction) is performed and read by trained medical personnel at screening to confirm eligibility. Additional cardiac assessment is recommended during grade 3 or 4 CRS for all subjects who require >1 fluid bolus for hypotension, who are transferred to the intensive care unit for hemodynamic management, or who require any dose of vasopressor for hypotension (Brudno and Kochenderfer, 2016).

Electrocardiogram

Twelve-lead electrocardiograms (ECGs) are obtained during screening, prior to LD chemotherapy on the first day of treatment, and prior to CTX112 administration on Day 1, and on Day 28. QTc and QRS intervals are determined from ECGs. Additional ECGs may be obtained.

Tumor Pathology and Biopsy

Tumor Pathology at Screening

Histopathological diagnosis of NHL subtype is based on local laboratory assessment. All subjects with disease amenable to biopsy (including SLL) undergo tumor biopsy during screening. However, if a biopsy of relapsed/refractory disease was performed after completion of the most recent line of therapy and within 3 months prior to enrollment, and an archival tissue sample is of sufficient volume and quality, archival tissue may be used in lieu of a biopsy at screening. Tumor biopsy refers to tumor tissue other than bone marrow, and whenever possible should not be performed on an identified target lesion. Bone biopsies and other decalcified tissues are not acceptable due to interference with downstream assays.

Portions of the tissue biopsy are submitted to a central laboratory for analysis. Archival tumor tissue samples may be analyzed for markers of aggressive NHL (e.g., MYC, BCL2, BCL6) as well as immune markers in the tumor and surrounding microenvironment (e.g., programmed cell death protein 1, programmed cell death-ligand 1).

Postinfusion Tumor Biopsy

To understand more about the trafficking of CTX112 into the tumor tissue and the impact of tumor environment on the function of CTX112, postinfusion tumor biopsies are obtained from subjects provided that the tumor is accessible and a biopsy is deemed safe by the treating physician. Tumor biopsy refers to tissue other than bone marrow.

For subjects with NHL, the postinfusion tumor biopsy is performed at Day 3 (within +5 days, although Day 3 is preferred). If disease progression or relapse occurs on study, every attempt should be made to obtain biopsy of the tumor and send to central pathology. For subjects with CLL/SLL, a postinfusion tumor biopsy is required only to confirm suspected disease progression or more aggressive histology (e.g., Richter's transformation).

Anytime tumor biopsy is performed, a sample (if available) should be sent to the central laboratory.

Brain MRI

To rule out CNS metastasis, a brain MRI is performed during the screening.

Lumbar Puncture

An LP is performed at screening according to institutional standard procedures in subjects with high risk for CNS involvement. These include subjects with high-grade B cell lymphoma with MYC and BCL2 and/or BCL6 rearrangement or double-expressor lymphoma (Riedell and Smith, 2018, Cancer 124, 4622-4632); subjects with testicular involvement of lymphoma; subjects with DLBCL treated with R-CHOP with high-risk scores on the CNS IPI, a tool used to estimate risk of CNS relapse/progression (Schmitz et al., 2016, J Clin Oncol 34, 3150-3156); or subjects with other disease histologies assessed as high risk for CNS involvement.

If clinically feasible, LPs is performed during neurotoxicity. CSF samples collected during neurotoxicity should be sent to the central laboratory for exploratory biomarkers and for presence of CTX112 (by PCR). Whenever LP is performed in the setting of neurotoxicity evaluation, in addition to the standard panel performed at the site (which should include at least cell count, Gram stain, and Neisseria meningitidis) the following viral panel must be performed: CSF PCR analysis for HSV-1 and -2, enterovirus, VZV, CMV, and HHV-6.

Results of viral panel should be available within 5 business days from draw to support appropriate management of a subject.

Excess sample (if available) is retained for exploratory biomarker research as described in this protocol.

Immune Effector Cell-Associated Encephalopathy (ICE) Assessment

Neurocognitive assessment is performed using the ICE assessment. The ICE assessment (Table 17) examines various areas of cognitive function: orientation, naming, following commands, writing, and attention.

The ICE assessment is performed at screening, before administration of CTX112 on Day 1, and on Days 2, 3, 5, 8, and 28. If a subject experiences CNS symptoms, the ICE assessment should continue to be performed approximately every 2 days until resolution of symptoms to grade 1 or baseline. To minimize variability, whenever possible the assessment should be performed by the same research staff member who is familiar with or trained in administration of the ICE assessment.

Patient-Reported Outcomes

In Phase 2 of the study, 2 PRO surveys, the Functional Assessment of Chronic Illness Therapy (FACIT) Measurement System's Functional Assessment of Cancer Therapy (FACT)-specific subscale for lymphoma (FACT-Lym v 4.0) or subscale for leukemia (FACT-Leu v 4.0), and the EuroQoL Group 5-Dimension (EQ-5D-5L) questionnaire is administered according to the schedules in Table 22 and Table 23.

FACT-Lym is a standardized tool based on the validated general FACT assessment in oncology, the FACT-G (Cella et al., 1993, J Clin Oncol 11, 570-579). The FACT-Lym evaluates the 4 domains of quality of life: physical, social/family, emotional, and functional well-being in the FACT-G, along with a 15-question subscale specific to lymphoma. The subscale component is designed to capture patient concerns and clinical issues specific to patients undergoing treatment for lymphoma (Cella et al., 2005, Blood 106, 750).

The FACT-Leu is a standardized tool also based on the validated general FACT assessment in oncology, the FACT-G Cella et al., 1993. In addition to the FACT-G subscales, it comprises a 17-question leukemia-specific subscale designed to capture concerns and symptoms specific to patients undergoing treatment for acute or chronic disease (Cella et al., 2012, Value Health 15, 1051-1058).

The EQ-5D-5L comprises a 5-item descriptive system questionnaire and a visual analogue scale (VAS). The descriptive system assesses health on the dimensions of mobility, self-care, usual activities, pain/discomfort, and anxiety/depression, with each dimension including 5 levels of severity: no problems, slight problems, moderate problems, severe problems, and extreme problems. The VAS assesses self-rated health on a 20-cm vertical scale from 0 (worst imaginable health) to 100 (best imaginable health).

Hospitalizations and Health Care Resource Utilization

Data on hospitalization, including intensive care unit stay and outpatient day use, is collected during the first 3 months after CTX112 infusion.

PET/CT and Radiologic Disease Response Assessments

Subjects with NHL

PET/CT (CT must include IV contrast) scans of all sites of disease (including the neck, chest, abdomen, and pelvis) are required. The CT portion of PET/CT should be diagnostic quality, or a standalone CT with IV contrast should be performed. MRI with contrast is allowed if CT is clinically contraindicated or as required by local regulation.

The baseline PET/CT (with IV contrast) must be performed within 28 days prior to administration of CTX112, and postinfusion scans are conducted per the schedule of assessments in Tables 22 and 23. Additional imaging at Month 2 is allowed. If a subject has symptoms consistent with possible disease progression, an unscheduled PET/CT (with IV contrast) should be performed.

Note that for subjects who receive an additional CTX112 infusion after PD, disease response is assessed relative to the most recent PET/CT scan and bone marrow prior to the additional infusion.

When possible, the imaging modalities, machines, and scanning parameters used to acquire PET/CT should be kept consistent during the study. All radiological images available at the site are transmitted by the sites to a central imaging vendor designated.

Tumor burden is quantified at baseline according to Lugano criteria. Tumor burden assessments are to include the sum of the products of diameters (SPD) calculated by aggregating the dimensions of each target lesion (nodal or extranodal) for a maximum of 6 target lesions, by multiplying the 2 longest perpendicular diameters of lesions. Target lesions should be selected from those with the largest size that can be reproducibly measured, representing overall tumor burden across multiple sites and organs.

For subjects with NHL, tumor response is determined according to Lugano criteria and is assessed (all parts of the study) and a central imaging review (only Phase 1 Part B and Phase 2).

TABLE 25
Lugano Classification Assessment Components

| Diagnostic CT/MRI | F¹⁸ FDG-PET | |
|---|---|---|
| Target Lymph Nodes and Extra Nodal Lesions | 5 Point Scale (Deauville) PET Score | |
| Up to 6 of the largest target nodes, nodal | (Lymph Nodes and Extra Lymphatic Sites)¹ | |
| masses, or other lymphomatous lesions that are | The 5-point scale scores the site of the most | |
| measurable in 2 diameters (longest diameter | intense FDG uptake for the time point, as follows: | |
| [LDi] and shortest diameter) should be identified | Score | Criteria |
| from different body regions representative of the | 1 | No uptake |
| subject's overall disease burden and include | 2 | Uptake ≤ mediastinum |
| mediastinal and retroperitoneal disease, if | 3 | Uptake > mediastinum but ≤ liver |
| involved. | 4 | Uptake moderately higher than liver |
| Nodal disease: Must have an LDi >1.5 cm | | (moderately indicates uptake greater |
| Extranodal disease: Must have an LDi >1.0 cm | | than normal liver) |
| Nonmeasured Lesions | 5 | Uptake markedly higher than liver |
| All other lesions (including nodal, extranodal, | | (markedly indicates much higher than |
| and assessable disease) should be followed as | | normal liver) |
| nonmeasured disease (e.g., cutaneous, GI, bone, | | and/or |
| spleen, liver, kidneys, pleural or pericardial | | New lesions |
| effusions, ascites). | X | New areas of uptake unlikely to be |
| Organ Enlargement (Spleen) | | related to lymphoma |

| | |
|---|---|
| The spleen is considered enlarged | Bone Marrow: FDG uptake assessed as |
| (splenomegaly) when >13 cm in the cranial to | No FDG uptake consistent with lymphoma |
| caudal dimension. | Focal FDG uptake consistent with lymphoma |
| New Lesions | Diffuse FDG uptake consistent with lymphoma |
| Nodal disease: Must have an LDi >1.5 cm | |
| Extranodal disease: Any size | |

CT: computed tomography; F¹⁸ FDG: fluorodeoxyglucose F18; LDi: longest diameter; MRI: magnetic resonance imaging; PET: positron emission tomography.
See (Barrington et al., 2014, J Clin Oncol 32, 3048-3058)

At each follow-up time point, a PET-based response and a CT-based response is made per the definitions in Table 26 below.

TABLE 26
Lugano Criteria for Response Assessment

| | | |
|---|---|---|
| Response | | |
| and Site | PET-based Response | CT-based Response |

COMPLETE

| | Complete Metabolic Response¹ | Complete Radiologic Response |
|---|---|---|
| | ALL of the following | ALL of the following |
| Lymph nodes, | Score of 1, 2, or 3¹ | Lymph nodes: All <1.5 cm in longest |
| extranodal | | diameter |
| lesions | | Extralymphatic disease absent |
| Nonmeasured | N/A | Absent |
| lesion | | |
| Organ | N/A | Spleen: normal size |
| enlargement | | |
| New | No new metabolically active lesions | None |
| lesions | (new lesions drive score 5) | |
| Bone | No FDG-avid disease in marrow | Normal by morphology; if |
| marrow | | indeterminate, IHC negative |

PARTIAL

| | | Partial Remission |
|---|---|---|
| | Partial Metabolic Response | ALL of the following |
| Lymph nodes, | Score of 4, or 5 with reduced uptake | ≥50% decrease in SPD of all |
| extranodal | from baseline and residual masses of | target lesions from baseline |
| lesions | any size | |
| Nonmeasured | N/A | Absent, normal, or regressed, |
| lesion | | but no increase |
| Organ | N/A | Spleen: ≥50% decrease from |
| enlargement | | baseline in enlarged portion |
| New | None | None |
| lesions | | |
| Bone | Residual uptake higher than uptake | N/A |
| marrow | in normal marrow but reduced | |
| | compared with baseline (e.g., | |
| | persistent focal changes in the | |
| | marrow with nodal response) | |

NO RESPONSE/STABLE DISEASE

| | No Metabolic Response | Stable Disease |
|---|---|---|
| Lymph nodes, | Score of 4, or 5 with no significant | <50% decrease in SPD of all target |
| extranodal | change in FDG uptake from baseline | lesion from baseline |
| lesions | | No progression |
| Nonmeasured | N/A | No increase consistent with |
| lesion | | progression |
| Organ | N/A | Spleen: No increase |
| enlargement | | consistent with progression |
| New lesions | None | None |
| Bone marrow | No change from baseline | N/A |

PROGRESSION

| | | Progressive Disease |
|---|---|---|
| | Progressive Metabolic Response | ANY of the following |
| Lymph nodes, | Lymph nodes/nodal masses: Score of | PPD Progression |
| extranodal | 4 or 5 with increased uptake | An individual node/extranodal lesion |
| lesions | compared to baseline | must be abnormal (nodal disease with |
| | Extranodal lesions: New FDG-avid | LDi >1.5 cm, extranodal disease with |
| | foci consistent with lymphoma | and LDi >1.0 cm) with: |
| | | Increase of ≥50% from the product of |
| | | the perpendicular diameters (PPD) |
| | | from nadir AND |
| | | Increase in LDi or SDi from nadir |
| | | ≥0.5 cm for lesions ≤2 cm |
| | | ≥1.0 cm for lesions >2 cm |
| Nonmeasured | None | Unequivocal progression |
| lesion | | |
| Organ | None | Progression of pre-existing |
| enlargement | | splenomegaly: Splenic length must |
| | | increase by 50% of the extent of its |
| | | prior increase beyond baseline (e.g., a |
| | | 15-cm spleen must increase to 16 cm). |
| | | New splenomegaly: Spleen must |
| | | increase by at least 2 cm from baseline |
| | | Or |
| | | Recurrent splenomegaly |
| New | New FDG-avid foci consistent with | Regrowth of previously resolved |
| lesions | lymphoma rather than another | lesions |
| | etiology | New node >1.5 cm in any axis |
| | | New extranodal site >1.0 cm in any |
| | | axis |
| | | New extranodal site <1.0 cm in any |
| | | axis or unequivocal/attributable to |
| | | lymphoma |
| | | New assessable disease unequivocal/ |
| | | attributable to lymphoma of any size |
| Bone | New/recurrent FDG-avid foci | New or recurrent involvement |
| marrow | | |

FDG: fluorodeoxyglucose; IHC: immunohistochemistry; LDi: longest diameter; N/A: not applicable; PPD: perpendicular diameters; SDi: shortest diameter; SPD: sum of the products of diameters.
Note:
It is recognized that in Waldeyer's ring or extranodal sites with high physiologic uptake or with activation within spleen or marrow (e.g., with chemotherapy or myeloid colony-stimulating factors), uptake may be greater than normal mediastinum and/or liver. In this circumstance, complete metabolic response may be inferred if uptake at sites of initial involvement is no greater than surrounding normal tissue even if the tissue has high physiologic uptake.
Deauville score of 3 represents a complete metabolic response (Barrington et al., 2014).

Subjects with CLL/SLL

For subjects with CLL/SLL, a CT (with IV contrast) of the neck, chest, abdomen, and pelvis is required at screening and at each disease response assessment (if indicated based on baseline evaluation; see Tables 22 and 23). If a subject has symptoms consistent with possible disease progression based on lymphadenopathy and/or splenomegaly/hepatomegaly, an unscheduled CT (with IV contrast) should be performed. In addition, imaging should be performed at the time of CR or PR. A CT of diagnostic quality performed as part of PET/CT is acceptable provided bidimensional modal and liver/spleen measurements are possible. After consultation with and agreement from the medical monitor, scans performed within 28 days prior to enrollment as part of standard of care may be used to satisfy screening requirements.

Note that for subjects who receive an additional CTX112 infusion after PD, disease response is assessed relative to the most recent PET/CT scan and bone marrow prior to the additional infusion.

When possible, the imaging modalities, machines, and scanning parameters used to acquire PET/CT should be kept consistent during the study. All radiological images available at the site are transmitted by the sites to a central imaging vendor designated.

For subjects with CLL/SLL, tumor response is determined according to iwCLL criteria. Response for all subjects is assessed (all phases of the study) and a central imaging review (only Phase 1 Part B and Phase 2). The determination of study eligibility and decisions regarding subject management and disease progression is made.

The International Workshop on CLL (iwCLL) Response Criteria (Hallek et al., 2018a, Blood 131, 2745-2760) provides a detailed description of the assessment of treatment response in subjects with CLL/SLL. To define response to therapy, 2 groups of parameters need to be assessed and documented: parameters of group A assess the lymphoid tumor load and constitutional symptoms; parameters of group B assess the hematopoietic system (Table 27).

TABLE 27
Response Definition after Treatment of Subjects With CLL/SLL

| Group | Parameter | CR | PR | PD | SD |
|---|---|---|---|---|---|
| A | Lymph nodes | None ≥1.5 cm | Decrease ≥50% | Increase ≥50% | Change |
| | | | (from baseline)* | from baseline or | of −49% |
| | | | | from response | to +49% |
| | Liver and/or | Spleen size | Decrease ≥50% | Increase ≥50% | Change |
| | spleen size† | <13 cm; liver | (from baseline) | from baseline or | of −49% |
| | | size normal | | from response | to +49% |
| | Constitutional | None | Any | Any | Any |
| | symptoms | | | | |
| | Circulating | Normal | Decrease ≥50% | Increase ≥50% | Change |
| | lymphocyte | | (from baseline) | from baseline | of −49% |
| | count | | | | to +49% |
| B | Platelet | ≥100 × 10⁹/L | ≥100 × 10⁹/L or | Decrease of | Change |
| | count | | increase ≥50% | ≥50% from | of −49% |
| | | | over baseline | baseline | to +49% |
| | | | | secondary | |
| | | | | to CLL | |
| | Hemoglobin | ≥11.0 g/dL | ≥11 g/dL or | Decrease of | Increase |
| | | (untransfused | increase ≥50% | ≥2 g/dL from | <11.0 g/dL |
| | | and without | over baseline | baseline | or <50% over |
| | | erythropoietin) | | secondary to | baseline, or |
| | | | | CLL | decrease <2 g/dL |
| | Marrow | Normocellular, | Presence of CLL | Increase of CLL | No change in |
| | | no CLL cells, | cells, or of B- | cells by ≥50% on | marrow |
| | | no B-lymphoid | lymphoid | successive | infiltrate |
| | | nodules | nodules, or not | biopsies | |
| | | | done | | |

*Sum of the products of 6 or fewer lymph nodes (as evaluated by CT scans and physical examination in clinical trials or by physical examination in general practice).
†Spleen size is considered normal if <13 cm. There is not firmly established international consensus of the size of a normal liver; therefore, liver size should be evaluated by imaging and manual palpation in clinical trials and be recorded according to the definition used in a study protocol.

CR (complete remission): all the criteria have to be met.
PD (progressive disease): at least 1 of the criteria of group A or group B must be met.
PR (partial response): for a PR, at least 2 of the parameters of group A and 1 parameter of group B need to improve if previously abnormal; if only 1 parameter of both groups A and B is abnormal before therapy, only 1 needs to improve.
SD (stable disease): all the criteria have to be met; constitutional symptoms alone do not define PD.
CR with Incomplete Bone Marrow Recovery (CRi)—Fulfills all requirements for CR except has persistent neutropenia, anemia, or thrombocytopenia thought to be unrelated to the disease and likely related to drug toxicity. These subjects must have a normal bone marrow aspirate and biopsy with no evidence of clonal infiltrates.

Criteria for B Symptoms

The presence of:
Unintentional weight loss ≥10% within the previous 6 months.
Significant fatigue (i.e., ECOG performance scale 2 or worse; cannot work or unable to perform usual activities).
Fevers ≥100.5° F. or 38.0° C. for 2 or more weeks without evidence of infection.
Night sweats for ≥1 month without evidence of infection

BALL Prognostic Score

The relapsed CLL BALL prognostic score (Soumerai et al., 2019, Lancet Haematol 6, e366-e374) consists of 4 factors, each assigned 1 point:
Beta-2 microglobulin (B2M) (serum) ≥5 mg/dL
Anemia: Hemoglobin <110 g/L for women or <120 g/L for men
Lactate dehydrogenase >upper limit of normal
Last therapy: Time from initiation of last therapy <24 months)

The relapsed CLL BALL score identifies 3 prognostic groups (low risk, score 0-1; intermediate risk, score 2-3; high risk, score 4) with significantly different overall survival. Application of this risk model to patients with relapsed/refractory CLL reliably identifies patients at increased risk of death.

Binet and Rai Staging System

TABLE 28
Binet Staging

| Stage | Clinical Features |
|---|---|
| A | Lymphocytosis, does not meet criteria for stages B or C |
| B | ≥3 areas of lymphadenopathy*, does not meet criteria for stage C |
| C | Anemia (Hb <10 g/dL) or thrombocytopenia (platelets <100 × |
| | 10⁹/L) |

*The 4 lymphadenopathy areas are: cervical, axillary, inguinal, spleen/liver
Adapted from (Binet et al., 1981, Cancer 48, 198-206).

TABLE 29
Modified Rai Clinical Stage

| Risk | |
|---|---|
| Category | Clinical Features |
| Low | Lymphocytes >15 × 10⁹/L |
| Intermediate | As 0 + lymphadenopathy or hepato- or splenomegaly |
| High | Anemia (Hb ≤11 g/dL) or thrombocytopenia |
| | (platelets ≤100 × 10⁹/L) |

Adapted from (Rai and Montserrat, 1987).

Bone Marrow Biopsy and Aspirate

Subjects with NHL

A bone marrow biopsy and aspirate are performed at screening and at Day 28 to evaluate extent of disease. A bone marrow biopsy obtained as part of standard of care within 28 days prior to enrollment may be used to satisfy screening requirements. Subjects with history of bone marrow involvement who achieve a CR as determined on PET/CT scan have a bone marrow biopsy to confirm response assessment. If a subject shows signs of relapse, the biopsy collection should be repeated. A sample of aspirate for presence of CTX112 should be sent for central laboratory evaluation at any point when bone marrow analysis is performed. Standard institutional guidelines for the bone marrow biopsy should be followed. Excess sample (if available) is retained for exploratory biomarker research as described in this protocol.

Subjects with CLL/SLL

A bone marrow biopsy and aspirate are performed at screening. A bone marrow biopsy obtained as part of standard of care within 28 days prior to enrollment may be used to satisfy screening requirements. Baseline samples are assessed locally to confirm disease pathology, evaluate extent of disease, and assign cohorts.

Subjects with CR/complete remission with incomplete bone marrow recovery (CRi) per clinical and lab assessments must have response confirmed by central laboratory evaluation of bone marrow aspirate and biopsy. In addition, minimal residual disease (MRD) assessment of bone marrow aspirate samples should be performed.

Bone marrow biopsy and aspirate may also be performed to assess cytopenia of uncertain cause (e.g., bone marrow toxicity, disease progression). For any BM aspirates collected, samples should be sent for CTX112 levels and/or other exploratory analyses, including for assessment of MRD in Phase 1 Part B and Phase 2.

Standard institutional guidelines for the bone marrow biopsy should be followed. Excess sample (if available) is retained for exploratory biomarker research as described in this protocol.

Minimal Residual Disease Assessments—Subjects with CLJSLL

In Phase 1 Part B and Phase 2, subjects with CLL or SLL have blood and bone marrow aspirate samples collected at the timepoints specified in the schedule of assessments in Table 22 for next-generation sequencing analysis at a central laboratory. In addition, local MRD assessment (in either peripheral blood and/or bone marrow by flow cytometry and/or next-generation sequencing) should be performed as clinically indicated or per institutional practices. If a local MRD assessment is performed at any time on the study, it should be recorded in the electronic data capture system.

Additional MRD blood and BM aspirate samples should be collected at the timepoints specified in the schedule of assessments in Table 22 for subjects at time of CR/CRi and for subjects with no evidence of CLL in peripheral blood or BM but who had a PR based on residual lymphadenopathy or splenomegaly at Months 6, 9, 12, 15, 18, and 24.

Excess sample (if available) is stored for exploratory research.

B Symptoms for Subjects with CLL/SLL

The absence of B symptoms (disease-related constitutional symptoms) is assessed for subjects with CLL/SLL according to the schedules of assessments in Tables 22 and 23. Assessment of B symptoms includes measurement of weight and temperature, assessment of fatigue via ECOG performance score, and presence of night sweats.

Laboratory Tests

Laboratory samples are collected and analyzed according to the schedule of assessment in Tables 22 and 23. Local laboratories meeting Clinical Laboratory Improvement Amendments requirements are utilized to analyze all tests listed in Table 30 according to standard institutional procedures.

TABLE 30
Local Laboratory Tests

| | |
|---|---|
| CBC with | Hematocrit, hemoglobin, red blood cell count, white |
| differential | blood cell count, reticulocyte, platelet count, and |
| | differential leukocyte count, including both percent |
| | and absolute number of lymphocytes |
| Lymphocyte | 6-color TBNK or equivalent commonly staining T |
| Subset Panel | cells (CD3, CD4, CD8), and NK cells (CD56, CD16) |
| B cells | CD19, CD20 |
| Serum | ALT (SGPT), AST (SGOT), total bilirubin, albumin, |
| Chemistry | alkaline phosphatase, bicarbonate, blood urea nitrogen, |
| | calcium, chloride, creatinine, eGFR, glucose, lactate |
| | dehydrogenase, magnesium, phosphorus, potassium, |
| | sodium, total protein |
| CRS and HLH | CRP, ferritin |
| Monitoring | |
| Coagulation | Prothrombin time, activated partial thromboplastin |
| Parameters | time, international normalized ratio, fibrinogen |
| Infectious Disease | HIV-1, HIV-2, hepatitis C virus antibody and PCR, |
| Testing/Viral | hepatitis B surface antigen, hepatitis B surface |
| Serology | antibody, hepatitis B core antibody |
| Immunoglobulins | IgA, IgG, IgM |
| Serum or Urine | Human chorionic gonadotropin |
| Pregnancy¹ | |

ALT: alanine aminotransferase; AST: aspartate aminotransferase; CRP: C-reactive protein; CRS: cytokine release syndrome; eGFR: estimated glomerular filtration rate; HIV-1/-2: human immunodeficiency virus type 1 or 2; HLH: hemophagocytic lymphohistiocytosis; HSCT: hematopoietic stem cell transplant; IgA/G/M: immunoglobulin A, G, or M; LD: lymphodepleting; NK: natural killer; PCR: polymerase chain reaction; SGOT: serum glutamic oxaloacetic transaminase; SGPT: serum glutamic pyruvic transaminase; TNBK: T, B, and NK cells.
¹For females of childbearing potential only. Serum pregnancy test required at screening. Serum or urine pregnancy test within 72 hours of start of LD chemotherapy.

CLL/SLL Disease Risk Labs

Local assessment for CLL/SLL risk factors should be performed at screening. These include an assessment of chromosomal abnormalities by conventional karyotyping and fluorescence in situ hybridization panel, including, at minimum, del 13q, del11q, del17p, and add12.

TP53 mutational status in peripheral blood should be assessed by next-generation sequencing at screening.

IGHV mutational status by gene sequencing should be assessed if historical results are not available.

Biomarkers

Blood, bone marrow, tumor, and CSF samples (only in subjects with ICANS) are collected to identify biomarkers that may be indicative of clinical response, resistance, safety, pharmacodynamic activity, or the mechanism of action of CTX112.

The labs in the following sections are submitted for analysis at a central laboratory. Excess sample (if available) is retained for exploratory biomarker research as described in this protocol.

CTX112 Pharmacokinetic Analysis

PK analysis of CTX112 cells is performed on blood samples collected according to the schedule described in Tables 22 and 23. In subjects experiencing signs or symptoms of CRS, HLH, or ICANS, additional blood samples should be drawn. The time course of the disposition of CTX112 in blood is described using a PCR assay that measures copies of CAR construct per g DNA. Complementary analyses using flow cytometry to confirm the presence of CAR protein on the cellular surface may also be performed.

The trafficking of CTX112 in CSF, bone marrow, or tumor tissues may be evaluated in any of these samples collected as per protocol-specific sampling.

Cytokines

Cytokines, including, at a minimum, interferon γ, IL-2, IL-2RA, IL-6, IL-8, IL-10, IL-15, tumor necrosis factor α, and IP-10, are analyzed in a central laboratory. Correlational analysis performed in multiple prior CAR T cell clinical studies have identified these cytokines, and others, as potential predictive markers for severe CRS and/or neurotoxicity (Wang and Han, 2018). Blood for cytokines is collected at specified times as described in Table 22. In subjects experiencing signs or symptoms of CRS, HLH, or neurotoxicity, additional samples should be drawn.

Anti-CTX112 Antibody

The CAR construct is composed of a murine scFv. Blood is collected throughout the study to assess for potential immunogenicity, per Tables 22 and 23.

Exploratory Biomarker Research

Exploratory biomarker research may be conducted to identify molecular biomarkers and immunophenotypes that may be indicative or predictive of clinical response, resistance, safety, pharmacodynamic activity, and/or the mechanism of action of treatment.

Discontinuation of sample collection may be requested. Continue sample collection for all listed time points until otherwise instructed.

### 8. Safety, Adverse Events, and Study Oversight

Each subject is to be monitored for clinical and laboratory evidence of AEs on a routine basis throughout the study. Details will be assessed and recorded, including the date of onset, event diagnosis (when known) or signs and symptoms, time course (end date, ongoing, intermittent), relationship to the study therapies or procedure, action(s) taken, and outcome. AEs in response to a query, observed by site personnel, or reported spontaneously by the subject is recorded. Any AEs observed or reported by the subject are recorded in the subject's medical record in the electronic data capture system.

Adverse Events

An AE is any untoward medical occurrence in a patient or clinical investigation subject administered a pharmaceutical product and which does not necessarily have a causal relationship with this treatment. An AE can therefore be any unfavorable or unintended sign (including an abnormal laboratory finding, for example), symptom, or disease temporally associated with the use of a medicinal (investigational) product whether or not considered related to the medicinal (investigational) product [Guidelines for Good Clinical Practice (GCP) E6(R2)]. In clinical studies, an AE can include an undesirable medical condition occurring at any time, including baseline or washout periods, even if no study treatment has been administered.

Additional criteria defining an AE are described below.

The following are considered to be AEs:
Aggravation of a pre-existing disease or permanent disorder (any clinically significant worsening in the nature, severity, frequency, or duration of a pre-existing condition)
Events resulting from protocol-mandated procedures (e.g., complications from invasive procedures)

The following are not considered to be AEs:
Medical or surgical procedures including elective or preplanned procedures such as surgery, endoscopy, tooth extraction, and transfusion. These should be recorded in the relevant electronic case report form (eCRF).
   Note: An untoward medical event occurring during the prescheduled elective procedure or routinely scheduled treatment should be recorded as an AE or SAE.
Pre-existing diseases or conditions that do not worsen during or after administration of the investigational medicinal product
Hospitalization planned for study treatment infusion or observation
The malignancy under study or signs and symptoms associated with the disease (except signs and symptoms of disease progression that meet seriousness criteria within 3 months after CTX112 administration) as well as progression or relapse of the underlying malignancy

Adverse events can occur before, during, or after treatment, and can be either treatment-emergent (AEs that start or worsen on or after CTX112 infusion) or non-treatment-emergent. A non-treatment-emergent AE is any new sign or symptom, disease, or other untoward medical event that occurs after written informed consent has been obtained and before the subject has received CTX112.

Serious Adverse Event

An AE of any untoward medical consequence must be classified as an SAE if it meets any of the following criteria:
Results in death
Is life-threatening (i.e., an AE that places the subject at immediate risk of death)
Requires in-patient hospitalization or prolongs an existing hospitalization (hospitalizations for scheduled medical or surgical procedures or to conduct scheduled treatments do not meet these criteria)
Results in persistent or significant disability or incapacity
Results in a congenital anomaly or birth defect in the newborn
Other important/significant medical events. Important medical events that may not result in death, be life-threatening, or require hospitalization may be considered serious when, based upon appropriate medical judgment, they may jeopardize the patient or subject and may require medical or surgical intervention to prevent one of the outcomes listed in this definition.

Adverse Events of Special Interest

An AESI, whether serious or nonserious, is one of scientific and medical concern specific to the product or program for which ongoing monitoring and rapid communication may be appropriate.

Based on the reported clinical experience of autologous CAR T cells, considered to be in the same pharmacological class, the following AESIs have been identified:
CTX112 infusion reactions
Grade ≥3 infections
TLS
CRS
ICANS
B cell aplasia
Hemophagocytic lymphohistiocytosis
Hypogammaglobulinemia
GvHD
Secondary malignancy
Any new autoimmune disorder that is possibly related or related to CTX112.

Adverse Event Severity

AEs are graded according to CTCAE version 5.0, with the exception of CRS, neurotoxicity, and GvHD, which is graded according to the criteria described herein.

When a CTCAE grade or protocol-specified criteria are not available, the toxicity grading in Table 31 will be used.

TABLE 31
Adverse Event Severity

| | |
|---|---|
| Grade 1 | Mild; asymptomatic or mild symptoms; clinical or diagnostic |
| | observations only; intervention not indicated. |
| Grade 2 | Moderate; minimal, local, or noninvasive intervention |
| | indicated; limiting age-appropriate instrumental ADL.¹ |
| Grade 3 | Severe or medically significant but not immediately life- |
| | threatening; hospitalization or prolongation of hospitalization |
| | indicated; disabling; limiting self-care ADL.² |
| Grade 4 | Life-threatening consequences; urgent intervention indicated. |
| Grade 5 | Death related to AE. |

ADL: Activities of Daily Living; AE: adverse event.
¹Instrumental ADL refer to preparing meals, shopping for groceries or clothes, using the telephone, managing money, etc.
²Self-care ADL refer to bathing, dressing and undressing, feeding self, using the toilet, taking medications, and not bedridden.

Adverse Event Causality

The relationship between each AE and CTX112, LD chemotherapy, and any protocol-mandated study procedure (all assessed individually) shall be assessed. The assessment of relationship is made based on the following definitions: Related: There is a clear causal relationship between the study treatment or procedure and the AE.

Possibly related: There is some evidence to suggest a causal relationship between the study treatment or procedure and the AE, but alternative potential causes also exist.

Not related: There is no evidence to suggest a causal relationship between the study treatment or procedure and the AE.

Outcome

The outcome of an AE or SAE classified and reported as follows:
Fatal
Not recovered/not resolved
Recovered/resolved
Recovered/resolved with sequelae
Recovering/resolving
Unknown

### 9. Statistical Analysis

Study Objectives and Hypotheses

The primary objective of Phase 1 Parts A and B is to assess the safety of escalating doses of CTX112 in combination with LD chemotherapy in subjects with relapsed/refractory B cell malignancies to determine the recommended Phase 2 dose(s).

The primary objective of Phase 2 is to assess the efficacy of CTX112 in combination with LD chemotherapy in selected Phase 1 Part B cohorts at the recommended Phase 2 dose determined in Phase 1 Parts A and B.

Study Endpoints

Primary Endpoints

Phase 1: Incidence of AEs, defined as DLTs

Phase 2: Objective response rate (CRR) per the Lugano Response Criteria for malignant lymphoma or iwCLL 2018 Guidelines for CLL/SLL.

Secondary Efficacy Endpoints

Phase 1: Objective response rate (CR+PR) per the Lugano Response Criteria for malignant lymphoma or iwCLL 2018 Guidelines for CLL/SLL

All Phases:

Duration of response/remission (DOR) is reported only for subjects who have had objective response events. This is assessed using the time between the first objective response and the first disease progression or death due to any cause after the first objective response.

Duration of clinical benefit (DOCB) is calculated as the time between the first objective response and the last disease progression or death.

Treatment-failure-free survival (TFFS) is calculated as the time between the first CTX112 infusion and the last disease progression or death due to any cause.

Progression-free survival (PFS) is calculated as the time between the first CTX112 infusion and the first disease progression or death due to any cause.

Overall survival (OS) is calculated as the time between date of first CTX112 infusion and death due to any cause.

Time to response, defined as the time between the date of CTX112 infusion until first documented objective response (CR or PR)

For secondary efficacy endpoints DOR, DOCB, TFFS, and PFS, the subjects who have not progressed and are still on study at the data cutoff date is censored at their last response assessment date. For OS, the subjects who are alive at the data cutoff date is censored at their last date known to be alive.

Secondary Safety Endpoints

The frequency and severity of AEs and clinically significant laboratory abnormalities are summarized and reported according to CTCAE version 5.0, except for CRS (ASTCT criteria), neurotoxicity (ICANS and CTCAE v5.0), and GvHD (MAGIC criteria).

Pharmacokinetic Endpoints

Pharmacokinetic data includes levels of CTX112 in blood over time as assessed by a PCR assay that measures copies of CAR construct. Analysis of CTX112 in blood may also occur using flow cytometry that detects CAR protein on the cellular surface. Such analysis may be used to confirm the presence of CTX112 in blood and to further characterize other cellular immunophenotypes.

Exploratory Endpoints
Levels of CTX112 in tissues (trafficking of CTX112 in bone marrow, CSF, and/or tumor tissue may be evaluated in any samples collected per protocol-specific sampling)
Levels of cytokines in blood. Levels in other tissues may also be evaluated
Incidence of anti-CTX112 antibodies
Levels of B cells and Igs over time
Impact of anti-cytokine therapy on CTX112 proliferation, CRS, and response
Phase 1 Part B and Phase 2 only: for CLL/SLL subjects only, percentage of subjects who are negative for MRD
Incidence and outcomes of autologous or allogeneic HSCT following CTX112 therapy
Incidence and type of subsequent anticancer therapy
First subsequent therapy-free survival, defined as the time between date of first CTX112 infusion and date of first subsequent therapy or death due to any cause
Phase 2 only: Change over time in patient-reported outcomes (PROs).

Analysis Sets

The following analysis sets are evaluated and used for presentation of the data.

Phase 1 (Dose Escalation)

DLT evaluable set (DES): All subjects who receive CTX112 and complete the DLT evaluation period or discontinue early after experiencing a DLT. The DES are used for determination of the recommended Phase 2 dose.

### Phase 1 and Phase 2 (Dose Escalation, Dose Optimization and Cohort Expansion)

Enrolled set: All subjects enrolled in the study. The enrolled set is classified according to the assigned dose level of CTX112.

Treated set: All subjects who received any study treatment in the study. The subjects in the treated set are classified according to the received study treatment.

Full analysis set (FAS): All subjects who received CTX112 infusion. The subjects in FAS are classified according to the assigned dose level of CTX112. The FAS is the primary analysis set for clinical activity assessment.

Safety analysis set (SAS): All subjects who received CTX112 infusion. The subjects in SAS are classified according to the received dose level of CTX112. The SAS is the primary analysis set for the characterization of CTX112 safety profile.

Sample Size

The sample size in the dose escalation phase of the study (Part A) is up to approximately 30 subjects, depending on the number of dose levels and the occurrence of DLTs. The sample size in the dose optimization part of the study (Part B) is 36 to 90 subjects, depending on the number of cohorts and dose levels evaluated. No formal sample size assessment was performed, the sample sizes are deemed sufficient to address the primary objective for safety.

The sample size in the cohort expansion phase of the study (Phase 2) is up to approximately 120 subjects (up to 6 cohorts, each with up to 20 subjects). Final enrollment depends on the actual number of cohorts evaluated. With 20 subjects, if the observed ORR is 70%, the corresponding 2-sided 90% Clopper-Pearson exact confidence interval is 49.2% to 86.0%; if the observed CR rate is 50%, the corresponding 2-sided 90% Clopper-Pearson exact confidence interval is 30.2% to 69.8%. The sample size is considered sufficient to provide adequate precision to estimate response rates and assess if the efficacy results warrant further evaluation of CTX112.

Planned Method of Analyses

The primary analysis of efficacy is conducted after the last treated subject in Phase 2 has completed the 6-month disease response assessment, is lost to follow up, withdraws from the study, or dies, whichever occurs first. A final analysis occurs at the end of study.

Tabulations are produced for appropriate disposition, demographic, baseline, efficacy, and safety parameters. By-subject listings are provided for all data, unless otherwise specified.

Efficacy Analysis

Primary analysis of ORR is based on independent central review of disease assessments in the FAS. Sensitivity analyses of ORR are performed. For malignant lymphoma, Lugano Response Criteria (Cheson et al., 2014, J Clin Oncol official journal of the American Society of Clinical Oncologists 32, 3059-3068) is used to determine the response. For CLL/SLL, the iwCLL 2018 Guidelines are used.

Objective response rate is summarized as a proportion with exact 95% confidence intervals.

For time-to-event variables such as DOR, DOCB, TFFS, PFS, and OS, medians with 95% confidence intervals are calculated using Kaplan-Meier methods.

Safety Analysis

All subjects who receive CTX112 and/or LD chemotherapy are included in the SAS. Clinical AEs are graded according to CTCAE version 5, except for CRS, which is graded according to ASTCT (Lee et al., 2019, Blood Marrow Transplant 25, 625-638), neurotoxicity, which is graded according to ICANS (Lee et al., 2019), and GvHD, which is graded according to MAGIC criteria (Harris et al., 2016, Biol Blood Marrow Transplant 22, 4-10). AEs, SAEs, and AESIs are summarized and reported according to the collection by study time period described in Table 22.

Treatment-emergent adverse events are defined as AEs that start or worsen on or after CTX112 infusion.

Vital signs are summarized using descriptive statistics. Summary tables are prepared to examine the distribution of laboratory measures over time.

Frequencies of subjects experiencing at least 1 AE are reported by body system and preferred term according to Medical Dictionary for Regulatory Activities (MedDRA) terminology. Detailed information collected for each AE will include description of the event, duration, whether the AE was serious, intensity, relationship to the investigational product, action taken, clinical outcome, and whether or not it was a DLT. Emphasis in the analysis is placed on AEs classified as dose-limiting.

Pharmacokinetic and Pharmacodynamic Analyses

Incidence of anti-CTX112 antibodies, levels of CTX112 CAR⁺ T cells in blood, and levels of cytokines in serum is summarized.

Biomarker Analyses

Investigation of additional biomarkers may include assessment of blood cells, tumor cells, and other subject-derived tissue. These assessments may evaluate DNA, RNA, proteins, and other biologic molecules derived from those tissues. Such evaluations will inform understanding of factors related to subject's response to CTX112 and the mechanism of action of the investigational product.

Patient-Reported Outcomes

For Phase 2 subjects only, descriptive statistics are presented for PRO, both as reported and as change from baseline. Further analyses may be performed on an ad hoc basis.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

The term “about” as used herein means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Where particular values are described in the application and claims, unless otherwise stated, the term “about” is implicit and in this context means within an acceptable error range for the particular value.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A method for treating a B-cell malignancy, comprising:
(i) subjecting a human patient having a B-cell malignancy to a lymphodepletion (LD) treatment; and
(ii) administering to the human patient a first dose of a population of genetically engineered T cells after step (i),
wherein the population of genetically engineered T cells comprising T cells that comprise:
(a) a disrupted T cell receptor alpha chain constant region (TRAC) gene,
(b) a disrupted beta-2-microglobulin (β2M) gene,
(c) a disrupted Regnase-1 (Reg1) gene,
(d) a disrupted Transforming Growth Factor Beta Receptor II (TGFBRII) gene, and
(e) a nucleic acid encoding a chimeric antigen receptor (CAR) that binds human CD19 (anti-CD19 CAR),
wherein the anti-CD19 CAR comprises a single chain variable fragment (scFv) that binds CD19 (anti-CD19 scFv), a co-stimulatory domain of CD28, and a CD3ζ cytoplasmic signaling domain, the anti-CD19 scFv comprising a heavy chain variable region (V_{H}) that comprises the same heavy chain complementary determining regions (CDRs) as those in SEQ ID NO: 81; and (ii) a light chain variable region (V_{L}) that comprises the same light chain CDRs as those in SEQ ID NO: 82;
wherein the nucleic acid encoding the anti-CD19 CAR is inserted at the disrupted TRAC gene; and
wherein the first dose of the population of genetically engineered T cells is about 1.0×10⁷ to about 6.0×10⁸ CAR⁺ T cells.

2. The method of claim 1, wherein the lymphodepletion treatment in step (i) comprises co-administration to the human patient fludarabine at about 30 mg/m² and cyclophosphamide at about 500-750 mg/m² per day for three days,
wherein step (i) is performed about 2-7 days prior to step (ii), and/or
wherein the first dose of the population of genetically engineered T cells is about 3×10⁷, about 1×10⁸, about 2×10⁸, about 3×10⁸, about 4.5×10⁸, or about 6×10⁸ CAR+ T cells.

3. (canceled)

4. The method of claim 1, wherein prior to step (i), the human patient does not show one or more of the following features:
(a) significant worsening of clinical status,
(b) requirement for supplemental oxygen to maintain a saturation level of greater than 91%,
(c) uncontrolled cardiac arrhythmia,
(d) hypotension requiring vasopressor support,
(e) active infection, and
(f) grade ≥2 acute neurological toxicity, and/or
wherein after step (i) and prior to step (ii), the human patient does not show one or more of the following features:
(a) active uncontrolled infection;
(b) worsening of clinical status compared to the clinical status prior to step (i); and
(c) neurological toxicity known to increase risk of immune effector cell-associated neurotoxicity syndrome (ICANS).

5-6. (canceled)

7. The method of claim 1, further comprising:
(iii) administering to the human patient one or more subsequent doses of the population of genetically engineered T cells, each of which is preceded by an LD treatment, and/or
(iv) monitoring the human patient for development of acute toxicity after administration of the population of genetically engineered T cells; and
(v) managing the acute toxicity when occurs.

8. The method of claim 7, wherein the human patient achieves a partial response (PR) or a complete response (CR) to the first dose of the population of genetically engineered T cells, wherein the prior to step (iii), the human patient meets at least the following criteria:
(a) confirmation of CD19+ tumor at relapse;
(b) no prior art dose-limiting-toxicity (DLT);
(c) no prior grade ≥3 cytokine release syndrome (CRS) without resolution to grade ≤2 within 72 hours following the first dose;
(d) no prior graft-versus-host disease (GvHD) following the first dose; and
(e) no prior grade 4 ICANS following the first dose.

9. (canceled)

10. The method of claim 7, wherein the acute toxicity comprises tumor lysis syndrome (TLS), cytokine release syndrome (CRS), neurotoxicity, which optionally comprises ICANS, viral encephalitis, or a combination thereof, B cell aplasia, hemophagocytic lymphohistiocytosis (HLH), cytopenia, graft-versus-host disease (GvHD), hypertension, renal insufficiency, or a combination thereof.

11. The method of claim 1, wherein the human patient has a relapsed and/or refractory B-cell malignancy, and/or
wherein the B-cell malignancy is selected from the group consisting of follicular lymphoma (FL), which optionally is grade 1-3a, mantle cell lymphoma (MCL), marginal zone lymphoma (MZL), chronic lymphocytic leukemia (CLL) and/or small lymphocytic lymphoma (SLL), and a large B cell lymphoma (LBLC).

12. (canceled)

13. The method of claim 11, wherein the LBLC is diffuse large B cell lymphoma (DLBCL), which optionally is not otherwise specified (NOS), high-grade B cell lymphoma with MYC and BCL2 and/or BCL6 rearrangements, primary mediastinal large B cell lymphoma (PMBCL), transformed FL and grade 3b FL, transformed MCL, or transformed MZL.

14. The method of claim 1, wherein the human patient has:
(a) grade 1-3a FL and has progressed after two lines of systemic therapy or has early relapse, wherein the systemic therapy optionally comprises an anti-CD20 antibody;
(b) MZL and has relapsed and/or refractory disease after up to 5 prior lines of therapy, which comprise at least an anti-CD20 antibody;
(c) MCL and has relapsed and/or refractory disease after up to 5 prior lines of therapy, which comprise at least anthracycline- or bendamustine-containing chemotherapy, an anti-CD20 antibody, or a Bruton tyrosine kinase (BTK) inhibitor;
(d) CLL/SLL and progressed after at least 2 prior therapies comprising a BTK inhibitor and venetoclax;
(e) a high-grade LBCL and has 2 or more lines of prior therapy comprising an anti-CD20 antibody and an anthracycline-containing chemotherapy, wherein the high-grade LBCL is DLBCL NOS, high-grade B cell lymphoma with MYC and BCL2 and/or BCL6 rearrangements, grade3b FL, transformed FL, transformed MCL, transformed MZL, or PMBCL; optionally wherein the human patient has transformed FL and has received at least one line of chemotherapy after transformation to DLBCL; or
(f) DLBCL and has received a prior CD-19-directed autologous CAR-T cell therapy.

15. The method of claim 1, wherein
the human patient received no more than 7×10⁴ T cell receptor-positive (TCR⁺) cells/kg in each dose of the genetically engineered T cells.

16. The method of claim 1, wherein the anti-CD19 scFv comprises the V_{H} comprising the amino acid sequence of SEQ ID NO: 81 and the V_{L} comprising the amino acid sequence of SEQ ID NO: 82.

17. The method of claim 16, wherein the anti-CD19 scFv comprises the amino acid sequence of SEQ ID NO: 77.

18. The method of claim 1, wherein the anti-CD19 CAR comprises the amino acid sequence of SEQ ID NO: 74.

19. The method of claim 1, wherein a fragment comprising the nucleotide sequence of SEQ ID NO: 18 in the TRAC gene is deleted and replaced by the nucleic acid encoding the anti-CD19 CAR.

20. The method of claim 19, wherein the disrupted TRAC gene comprises the nucleotide sequence of SEQ ID NO: 90.

21. The method of claim 1, wherein the disrupted β2M gene in the T cells comprises one or more of the nucleotide sequences listed in Table 2;
wherein the disrupted Reg1 gene in the T cells comprises one or more of the nucleotide sequences listed in Table 4; and/or
wherein the disrupted TGFBRII gene in the T cells comprises one or more of the nucleotide sequences listed in Table 3.

22-23. (canceled)

24. The method of claim 1, wherein at least 30% of the T cells in the population express the anti-CD19 CAR, wherein at least 90% of the T cells in the population are TCR−, wherein at least 60% of the T cells in the population are β2M−, wherein at least 80% of the T cells in the population are TGFBRII−, and/or wherein at least 90% of the T cells in the population are Reg1−.

25. The method of claim 24, wherein the population of genetically engineered T cells comprises:
(a) at least 50% of the T cells express the anti-CD19 CAR;
(b) at least 99% of the T cells are TCR−;
(c) about 65% to about 80% of the T cells are β2M⁻;
(d) about 80% to about 90% of the T cells are TGFBRII⁻; and/or
(e) about 95% to about 97% of the T cells are Reg1⁻.

26. The method of claim 1, wherein the population of genetically engineered T cells is suspected in a solution comprising human serum albumin and a cryopreservative solution.

27. The method of claim 1, wherein the population of genetically engineered T cells comprise human primary T cells, or wherein the population of genetically engineered T cells is allogeneic to the human patient.

28. The method of claim 27, wherein the T cells are derived from one or more healthy human donors.

29. (canceled)


SEQUENCE LISTING (91 sequences)

SEQ ID NO: 1 - RNA, 100 nt, synthetic construct
  source 1..100: mol_type = other RNA
agagcaacagtgctgtggccgttttagagctagaaatagcaagttaaaataaggctagtccgttatcaacttgaaaaagtggcaccgagtcggtgctttt

SEQ ID NO: 2 - RNA, 100 nt, synthetic construct; identical to SEQ ID NO: 1
  source 1..100: mol_type = other RNA
  modified_base 1..4: mod_base = OTHER; note = modified with 2-O-methyl phosphorothioate
  modified_base 97..100: mod_base = OTHER; note = modified with 2-O-methyl phosphorothioate

SEQ ID NO: 3 - RNA, 20 nt, synthetic construct
  source 1..20: mol_type = other RNA
agagcaacagtgctgtggcc

SEQ ID NO: 4 - RNA, 20 nt, synthetic construct
  source 1..20: mol_type = other RNA
  misc_feature 1..4: note = modified with 2-O-methyl phosphorothioate
agagcaacagtgctgtggcc

SEQ ID NO: 5 - RNA, 100 nt, synthetic construct
  source 1..100: mol_type = other RNA
gctactctctctttctggccgttttagagctagaaatagcaagttaaaataaggctagtccgttatcaacttgaaaaagtggcaccgagtcggtgctttt

SEQ ID NO: 6 - RNA, 100 nt, synthetic construct; identical to SEQ ID NO: 5
  source 1..100: mol_type = other RNA
  modified_base 1..4: mod_base = OTHER; note = modified with 2-O-methyl phosphorothioate
  modified_base 97..100: mod_base = OTHER; note = modified with 2-O-methyl phosphorothioate

SEQ ID NO: 7 - RNA, 20 nt, synthetic construct
  source 1..20: mol_type = other RNA
gctactctctctttctggcc

SEQ ID NO: 8 - RNA, 20 nt, synthetic construct
  source 1..20: mol_type = other RNA
  modified_base 1..4: mod_base = OTHER; note = modified with 2-O-methyl phosphorothioate
gctactctctctttctggcc

SEQ ID NO: 9 - RNA, 100 nt, synthetic construct
  source 1..100: mol_type = other RNA
acgacgcgtgggtggcaagcgttttagagctagaaatagcaagttaaaataaggctagtccgttatcaacttgaaaaagtggcaccgagtcggtgctttt

SEQ ID NO: 10 - RNA, 100 nt, synthetic construct; identical to SEQ ID NO: 9
  source 1..100: mol_type = other RNA
  modified_base 1..4: mod_base = OTHER; note = modified with 2-O-methyl phosphorothioate
  modified_base 97..100: mod_base = OTHER; note = modified with 2-O-methyl phosphorothioate

SEQ ID NO: 11 - RNA, 20 nt, synthetic construct
  source 1..20: mol_type = other RNA
acgacgcgtgggtggcaagc

SEQ ID NO: 12 - RNA, 20 nt, synthetic construct
  source 1..20: mol_type = other RNA
  modified_base 1..4: mod_base = OTHER; note = modified with 2-O-methyl phosphorothioate
acgacgcgtgggtggcaagc

SEQ ID NO: 13 - RNA, 100 nt, synthetic construct
  source 1..100: mol_type = other RNA
cccctaccatgactttattcgttttagagctagaaatagcaagttaaaataaggctagtccgttatcaacttgaaaaagtggcaccgagtcggtgctttt

SEQ ID NO: 14 - RNA, 100 nt, synthetic construct; identical to SEQ ID NO: 13
  source 1..100: mol_type = other RNA
  modified_base 1..4: mod_base = OTHER; note = modified with 2-O-methyl phosphorothioate
  modified_base 97..100: mod_base = OTHER; note = modified with 2-O-methyl phosphorothioate

SEQ ID NO: 15 - RNA, 20 nt, synthetic construct
  source 1..20: mol_type = other RNA
cccctaccatgactttattc

SEQ ID NO: 16 - RNA, 20 nt, synthetic construct
  source 1..20: mol_type = other RNA
  modified_base 1..4: mod_base = OTHER; note = modified with 2'-O-methyl phosphorothioate
cccctaccatgactttattc

SEQ ID NO: 17 - DNA, 23 bp, synthetic construct
  source 1..23: mol_type = other DNA
agagcaacagtgctgtggcctgg

SEQ ID NO: 18 - DNA, 20 bp, synthetic construct
  source 1..20: mol_type = other DNA
agagcaacagtgctgtggcc

SEQ ID NO: 19 - DNA, 23 bp, synthetic construct
  source 1..23: mol_type = other DNA
gctactctctctttctggcctgg

SEQ ID NO: 20 - DNA, 20 bp, synthetic construct
  source 1..20: mol_type = other DNA
gctactctctctttctggcc

SEQ ID NO: 21 - DNA, 23 bp, synthetic construct
  source 1..23: mol_type = other DNA
acgacgcgtgggtggcaagcggg

SEQ ID NO: 22 - DNA, 20 bp, synthetic construct
  source 1..20: mol_type = other DNA
acgacgcgtgggtggcaagc

SEQ ID NO: 23 - DNA, 23 bp, synthetic construct
  source 1..23: mol_type = other DNA
cccctaccatgactttattctgg

SEQ ID NO: 24 - DNA, 20 bp, synthetic construct
  source 1..20: mol_type = other DNA
cccctaccatgactttattc

SEQ ID NO: 25 - RNA, 100 nt, synthetic construct
  source 1..100: mol_type = other RNA
nnnnnnnnnnnnnnnnnnnngttttagagctagaaatagcaagttaaaataaggctagtccgttatcaacttgaaaaagtggcaccgagtcggtgctttt

SEQ ID NO: 26 - RNA, 96 nt, synthetic construct
  source 1..96: mol_type = other RNA
nnnnnnnnnnnnnnnnnnnngttttagagctagaaatagcaagttaaaataaggctagtccgttatcaacttgaaaaagtggcaccgagtcggtgc

SEQ ID NO: 27 - RNA, 114 nt, synthetic construct
  source 1..114: mol_type = other RNA
  variation 108..114: note = u or absent
  variation 18..30: note = n is a, c, g, u, or absent
nnnnnnnnnnnnnnnnnnnnnnnnnnnnnngttttagagctagaaatagcaagttaaaataaggctagtccgttatcaacttgaaaaagtggcaccgagtcggtgctttttttt

SEQ ID NO: 28 - protein, 1368 aa, synthetic construct [sequence omitted]

SEQ ID NO: 29 - DNA, 79 bp, synthetic construct
  source 1..79: mol_type = other DNA
cgtggccttagctgtgctcgcgctactctctctttctgcctggaggctatccagcgtgagtctctcctaccctcccgct

SEQ ID NO: 30 - DNA, 78 bp, synthetic construct
  source 1..78: mol_type = other DNA
cgtggccttagctgtgctcgcgctactctctctttcgcctggaggctatccagcgtgagtctctcctaccctcccgct

SEQ ID NO: 31 - DNA, 75 bp, synthetic construct
  source 1..75: mol_type = other DNA
cgtggccttagctgtgctcgcgctactctctctttctggaggctatccagcgtgagtctctcctaccctcccgct

SEQ ID NO: 32 - DNA, 84 bp, synthetic construct
  source 1..84: mol_type = other DNA
cgtggccttagctgtgctcgcgctactctctctttctggatagcctggaggctatccagcgtgagtctctcctaccctcccgct

SEQ ID NO: 33 - DNA, 55 bp, synthetic construct
  source 1..55: mol_type = other DNA
cgtggccttagctgtgctcgcgctatccagcgtgagtctctcctaccctcccgct

SEQ ID NO: 34 - DNA, 82 bp, synthetic construct
  source 1..82: mol_type = other DNA
cgtggccttagctgtgctcgcgctactctctctttctgtggcctggaggctatccagcgtgagtctctcctaccctcccgct

SEQ ID NO: 35 - DNA, 13 bp, synthetic construct
  source 1..13: mol_type = other DNA
catgactggaaga

SEQ ID NO: 36 - DNA, 16 bp, synthetic construct
  source 1..16: mol_type = other DNA
catgacttctggaaga

SEQ ID NO: 37 - DNA, 17 bp, synthetic construct
  source 1..17: mol_type = other DNA
catgactttctggaaga

SEQ ID NO: 38 - DNA, 21 bp, synthetic construct
  source 1..21: mol_type = other DNA
catgactttatttctggaaga

SEQ ID NO: 39 - DNA, 21 bp, synthetic construct
  source 1..21: mol_type = other DNA
catgactttaattctggaaga

SEQ ID NO: 40 - DNA, 18 bp, synthetic construct
  source 1..18: mol_type = other DNA
catgacttttctggaaga

SEQ ID NO: 41 - DNA, 11 bp, synthetic construct
  source 1..11: mol_type = other DNA
catctggaaga

SEQ ID NO: 42 - DNA, 19 bp, synthetic construct
  source 1..19: mol_type = other DNA
catgactttttctggaaga

SEQ ID NO: 43 - DNA, 19 bp, synthetic construct
  source 1..19: mol_type = other DNA
catgactttatctggaaga

SEQ ID NO: 44 - DNA, 13 bp, synthetic construct
  source 1..13: mol_type = other DNA
catgactttaaga

SEQ ID NO: 45 - DNA, 10 bp, synthetic construct
  source 1..10: mol_type = other DNA
ttctggaaga

SEQ ID NO: 46 - DNA, 18 bp, synthetic construct
  source 1..18: mol_type = other DNA
catgactttactggaaga

SEQ ID NO: 47 - DNA, 21 bp, synthetic construct
  source 1..21: mol_type = other DNA
gtgggtggcaaagcgggtggt

SEQ ID NO: 48 - DNA, 19 bp, synthetic construct
  source 1..19: mol_type = other DNA
gtgggtggcagcgggtggt

SEQ ID NO: 49 - DNA, 21 bp, synthetic construct
  source 1..21: mol_type = other DNA
gtgggtggcatagcgggtggt

SEQ ID NO: 50 - DNA, 18 bp, synthetic construct
  source 1..18: mol_type = other DNA
gtgggtggagcgggtggt

SEQ ID NO: 51 - protein, 22 aa, synthetic construct
MLLLVTSLLLCELPHPAFLLIP

SEQ ID NO: 52 - protein, 21 aa, synthetic construct
MALPVTALLLPLALLLHAARP

SEQ ID NO: 53 - protein, 23 aa, synthetic construct
IYIWAPLAGTCGVLLLSLVITLY

SEQ ID NO: 54 - DNA, 126 bp, synthetic construct
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 55 - protein, 42 aa, synthetic construct
KRGRKKLLYIFKQPFMRPVQTTQEEDGCSCRFPEEEEGGCEL

SEQ ID NO: 56 - DNA, 120 bp, synthetic construct
  source 1..120: mol_type = other DNA
tcaaagcggagtaggttgttgcattccgattacatgaatatgactcctcgccggcctgggccgacaagaaaacattaccaaccctatgcccccccacgagacttcgctgcgtacaggtcc

SEQ ID NO: 57 - protein, 40 aa, synthetic construct
SKRSRLLHSDYMNMTPRRPGPTRKHYQPYAPPRDFAAYRS

SEQ ID NO: 58 - DNA, 336 bp, synthetic construct
  source 1..336: mol_type = other DNA
cgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 59 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - protein, 11 aa, synthetic construct
RASQDISKYLN

SEQ ID NO: 61 - protein, 7 aa, synthetic construct
HTSRLHS

SEQ ID NO: 62 - protein, 9 aa, synthetic construct
QQGNTLPYT

SEQ ID NO: 63 - protein, 5 aa, synthetic construct
DYGVS

SEQ ID NO: 64 - protein, 16 aa, synthetic construct
VIWGSETTYYNSALKS

SEQ ID NO: 65 - protein, 12 aa, synthetic construct
HYYYGGSYAMDY

SEQ ID NO: 66 - protein, 11 aa, synthetic construct
RASQDISKYLN

SEQ ID NO: 67 - protein, 7 aa, synthetic construct
HTSRLHS

SEQ ID NO: 68 - protein, 9 aa, synthetic construct
QQGNTLPYT

SEQ ID NO: 69 - protein, 7 aa, synthetic construct
GVSLPDY

SEQ ID NO: 70 - protein, 5 aa, synthetic construct
WGSET

SEQ ID NO: 71 - protein, 12 aa, synthetic construct
HYYYGGSYAMDY

SEQ ID NO: 72 - DNA, 1518 bp, synthetic construct
  source 1..1518: mol_type = other DNA
atgcttcttttggttacgtctctgttgctttgcgaacttcctcatccagcgttcttgctgatccccgatattcagatgactcagaccaccagtagcttgtctgcctcactgggagaccgagtaacaatctcctgcagggcaagtcaagacattagcaaatacctcaattggtaccagcagaagcccgacggaacggtaaaactcctcatctatcatacgtcaaggttgcattccggagtaccgtcacgattttcaggttctgggagcggaactgactattccttgactatttcaaacctcgagcaggaggacattgcgacatatttttgtcaacaaggtaataccctcccttacactttcggaggaggaaccaaactcgaaattaccgggtccaccagtggctctgggaagcctggcagtggagaaggttccactaaaggcgaggtgaagctccaggagagcggccccggtctcgttgcccccagtcaaagcctctctgtaacgtgcacagtgagtggtgtatcattgcctgattatggcgtctcctggataaggcagcccccgcgaaagggtcttgaatggcttggggtaatatggggctcagagacaacgtattataactccgctctcaaaagtcgcttgacgataataaaagataactccaagagtcaagttttccttaaaatgaacagtttgcagactgacgataccgctatatattattgtgctaaacattattactacggcggtagttacgcgatggattattgggggcaggggacttctgtcacagtcagtagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 73 - protein, 506 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 74 - protein, 484 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75 - protein, 484 aa, synthetic construct; identical to SEQ ID NO: 74

SEQ ID NO: 76 - DNA, 735 bp, synthetic construct
  source 1..735: mol_type = other DNA
gatattcagatgactcagaccaccagtagcttgtctgcctcactgggagaccgagtaacaatctcctgcagggcaagtcaagacattagcaaatacctcaattggtaccagcagaagcccgacggaacggtaaaactcctcatctatcatacgtcaaggttgcattccggagtaccgtcacgattttcaggttctgggagcggaactgactattccttgactatttcaaacctcgagcaggaggacattgcgacatatttttgtcaacaaggtaataccctcccttacactttcggaggaggaaccaaactcgaaattaccgggtccaccagtggctctgggaagcctggcagtggagaaggttccactaaaggcgaggtgaagctccaggagagcggccccggtctcgttgcccccagtcaaagcctctctgtaacgtgcacagtgagtggtgtatcattgcctgattatggcgtctcctggataaggcagcccccgcgaaagggtcttgaatggcttggggtaatatggggctcagagacaacgtattataactccgctctcaaaagtcgcttgacgataataaaagataactccaagagtcaagttttccttaaaatgaacagtttgcagactgacgataccgctatatattattgtgctaaacattattactacggcggtagttacgcgatggattattgggggcaggggacttctgtcacagtcagtagt

SEQ ID NO: 77 - protein, 245 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 78 - DNA, 261 bp, synthetic construct
  source 1..261: mol_type = other DNA
gctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgc

SEQ ID NO: 79 - DNA, 252 bp, synthetic construct
  source 1..252: mol_type = other DNA
tttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgc

SEQ ID NO: 80 - protein, 84 aa, synthetic construct
FVPVFLPAKPTTTPAPRPPTPAPTIASQPLSLRPEACRPAAGGAVHTRGLDFACDIYIWAPLAGTCGVLLLSLVITLYCNHRNR

SEQ ID NO: 81 - protein, 120 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 82 - protein, 107 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 83 - protein, 18 aa, synthetic construct
GSTSGSGKPGSGEGSTKG

SEQ ID NO: 84 - DNA, 145 bp, synthetic construct
  source 1..145: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 85 - DNA, 130 bp, synthetic construct
  source 1..130: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 86 - DNA, 145 bp, synthetic construct
  source 1..145: mol_type = other DNA
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 87 - DNA, 141 bp, synthetic construct
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 88 - DNA, 800 bp, synthetic construct
  source 1..800: mol_type = other DNA
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttca

SEQ ID NO: 89 - DNA, 804 bp, synthetic construct
  source 1..804: mol_type = other DNA
tggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 90 - DNA, 1178 bp, synthetic construct
  source 1..1178: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 91 - DNA, 4358 bp, synthetic construct
  source 1..4358: mol_type = other DNA
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccaccatgcttcttttggttacgtctctgttgctttgcgaacttcctcatccagcgttcttgctgatccccgatattcagatgactcagaccaccagtagcttgtctgcctcactgggagaccgagtaacaatctcctgcagggcaagtcaagacattagcaaatacctcaattggtaccagcagaagcccgacggaacggtaaaactcctcatctatcatacgtcaaggttgcattccggagtaccgtcacgattttcaggttctgggagcggaactgactattccttgactatttcaaacctcgagcaggaggacattgcgacatatttttgtcaacaaggtaataccctcccttacactttcggaggaggaaccaaactcgaaattaccgggtccaccagtggctctgggaagcctggcagtggagaaggttccactaaaggcgaggtgaagctccaggagagcggccccggtctcgttgcccccagtcaaagcctctctgtaacgtgcacagtgagtggtgtatcattgcctgattatggcgtctcctggataaggcagcccccgcgaaagggtcttgaatggcttggggtaatatggggctcagagacaacgtattataactccgctctcaaaagtcgcttgacgataataaaagataactccaagagtcaagttttccttaaaatgaacagtttgcagactgacgataccgctatatattattgtgctaaacattattactacggcggtagttacgcgatggattattgggggcaggggacttctgtcacagtcagtagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtgtggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg